SEQUENCE LISTING (6 sequences)

SEQ ID NO: 1 - DNA, 1033 bp, Artificial Sequence
  Description of Artificial Sequence Synthetic lef-3 promoter sequence
ccgagaagaaggcggtttgtataaaacccatttttcgaaatggttaacaaacttgtttagcatttggatcgtttcgtgttcaaacgcgtcgaaaacttttaaaacgcaattgccgccgggacgcaggcaaattaaaattagctgcgtctcgcacatgatcaaatcaaagttgagacgttcttgttcgttttcgcgtccattaacgtcaaccgagccatctgccaacaccagatcgcacgcgttgccacacttgatgctaatctcaaatacaacatttttatcaaacacgtcgcctgacttgtcgggccccgtaatggttgtgaaatttttgcgtttgcgcactgtcggtttgtacacgcacaccgagttgtttgtcaacgtgacgccatacgctttgcaaagcgggttcaacgacatggtatagttggcaaactcgcccggtccgccgcacaaatccaaaaacgtgtcaacgtgtcggcaaacgtgaaactttttgtcgatctctgatagttttcgccaacatctaggtctgcgcgttgggcgtttgtcaaataattttgagcgagcgcaaaccaccgacttgctgctgaacgtgttcaaaccatctttgagtttatttaatttttgctgcaacatttttactcttcgtgtcggtcgcaatgtttgtgtcgaaaaagacggccaacacgctcagcaaaactatacaaataaagaacaaaaatacgtacgcaatattaacattgaccgtttgatcgttaaatcggacgggtctgttcagagccgctcttattctctcgttgtacattgttaaagtttttgtttttaaattgtacacaatcggcgtgttgtagtcgaaattttcaaaatcggctttttgaaacattgttctgaacgtgttgtcgagcggcgtgttgctggccacgtttataatcaactccctccacgctaacgaacggtgctctggcgacacttcgatttcgtcgccattcagtatttgccatcggatagattcccacatatcgacaacagcaat

SEQ ID NO: 2 - DNA, 1047 bp, Artificial Sequence
  Description of Artificial Sequence Synthetic TLP promoter sequence
tgctagcccaattggccactgttgtacgaaatatcgtcgtcaacgtgtttgaatacatgttggcccgtaccgttgggtaaatctatgcatctggagtcgccggaacactcgtactggttgtcagagtttctgatccggttgatgcacgttatcagttgtgactcgttattattcaaacatttgaaatattgcgtgtcgccgatatcggccgttatgtacgtgtgtccggcgccgttaaacgcgcacggatgcgcttccacgcacgacattaagttgcgatcaaatattttattcgcggggcattcgcccaccacgtggcgcccatttacgcactgcataaactggttgacgagcaaattggagggaaagtatgatagtatatagccgtctggcctgttttcacacaattcgttaactttacactggccggtttccgcgtcaaacgtgtaattatctggacattcttcgactgcgtgcgctccgtttgcaaaacacctaagatagaacgtgggatgatacaagtgcgcgttggtagaataatctttgtccaagtgttggttcaacaccaacgtgtccagcaaacgctcgtccatgggataaagaccggcagacttgttgtcgcacggcggcacgggaacacattttagttgtgcgtaatcaaagttaaaatatgcggggcatttcatggtcacgtcggccttgtcgccgctcaaaataaactcgttgggattttcatcatttgctctaacgcgatcgtgtacgattcgatcaacaggttgaaatttttgatttaagaaatcaaaaatttcaatccggtcatcatgcacgctttcgtgataggtggaaaggtcgacggtgttgaaccacgttacaatataagtgttttgcataatatccgacacgtagcctattacgtcgggtgtgggttcgtctgcgttggtgcgcttcacatattcagtcatcacttggagccgcttggtgaaagtcgtttcgtcaaattcaaaataaattgccaaatacattaaagtaaacgctattataagaaaa

SEQ ID NO: 3 - protein, 265 aa, Artificial Sequence
  Description of Artificial Sequence Synthetic beta-lactamase polypeptide
MGHPETLVKVKDAEDQLGARVGYIELDLNSGKILESFRPEERFPMMSTFKVLLCGAVLSRDDAGQEQLGRRIHYSQNDLVEYSPVTEKHLTDGMTVRELCSAAITMSDNTAANLLLTTIGGPKELTAFLHNMGDHVTRLDHWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 4 - DNA, 5515 bp, Artificial Sequence
  Description of Artificial Sequence Synthetic polynucleotide
aaacttaagacttgctctattacaattttatttgttggatcaattgcatatttaatattatacaattccataacgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaacaaatgactcatcaatcgatcgtgcgtacacgtagaattctactcgtaaagcgagtttatgagccgtgtgcaaaacatgacatcatctcgatttgaaaaacaaatgacatcatccactgatcgtgcgttacaagtagaattctactcgtaaagccagttcggttatgagccgtgtgcaaaacatgacatcagcttatgactcgtacttgattgtgttttacgcgtagaattctactcgtaaagccagttcaattttaaaaacaaatgacatcatccaaattaataaatgacaagcaatgacaaaataatattaggcaataaattttaacatttatttaattgtgtttaatattacatttttgttgagtgcactagtcagtgtggtggaattgcccttagaatttgtcgggtccattgtccgtgtgcgctaggtaccgagctcggatccactagtaacggccgccagtgtgctggaattcgcccttttgctagcccaattggccactgttgtacgaaatatcgtcgtcaacgtgtttgaatacatgttggcccgtaccgttgggtaaatctatgcatctggagtcgccggaacactcgtactggttgtcagagtttctgatccggttgatgcacgttatcagttgtgactcgttattattcaaacatttgaaatattgcgtgtcgccgatatcggccgttatgtacgtgtgtccggcgccgttaaacgcgcacggatgcgcttccacgcacgacattaagttgcgatcaaatattttattcgcggggcattcgcccaccacgtggcgcccatttacgcactgcataaactggttgacgagcaaattggagggaaagtatgatagtatatagccgtctggcctgttttcacacaattcgttaactttacactggccggtttccgcgtcaaacgtgtaattatctggacattcttcgactgcgtgcgctccgtttgcaaaacacctaagatagaacgtgggatgatacaagtgcgcgttggtagaataatctttgtccaagtgttggttcaacaccaacgtgtccagcaaacgctcgtccatgggataaagaccggcagacttgttgtcgcacggcggcacgggaacacattttagttgtgcgtaatcaaagttaaaatatgcggggcatttcatggtcacgtcggccttgtcgccgctcaaaataaactcgttgggattttcatcatttgctctaacgcgatcgtgtacgattcgatcaacaggttgaaatttttgatttaagaaatcaaaaatttcaatccggtcatcatgcacgctttcgtgataggtggaaaggtcgacggtgttgaaccacgttacaatataagtgttttgcataatatccgacacgtagcctattacgtcgggtgtgggttcgtctgcgttggtgcgcttcacatattcagtcatcacttggagccgcttggtgaaagtcgtttcgtcaaattcaaaataaattgccaaatacattaaagtaaacgctattataagaaaaaagcttggtaccgagctcggatccactagtaacggccgccagtgtgctggaattcgcccttcaccatggacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagatatcgaagggcgaattctgcagatatccatcacactggcggccgctcgagtctagaccccgagatccccgacgttcccggccttcgccgcagtcgcaagcagtaatcaaaacagcaaatcgacggttttgaaatactcgtacggctctttgaccaagtaataaaatgcaagcatcaaaaatattgcaaaatacacaaaaaacgtaagttccttgtgcgcaataaaggccgcaagggccaccgctgtatttgtcaaaaataaacccgctatcaccccattcaacttgttgttatttttgttcattgccaacaacgtgttttgcctgtaagtgtattgcataaactcgagacgtgtgtacagcgagctgctggccagcgcttggcccacgagcgtggcctcgtcgaaatctttgatctcaccggtttgtgtcacgtaggccagataacggtcgggtatataagatgcctcaatgctactagtaaatcagtcacaccaaggcttcaataaggaacacacaagcaagcccttgactcaaagggctgccgggctgcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgcgcagaactcgtggtgctgggcactgctgctgctgcggcagctggcaacctgacttgtatcgtcgcgatcggaaatgagaacaggggcatcttgagcccctgcggacggtgccgacaggttcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcccattattgaagcatttatcagggttattgtctcatgagcggatacatatttgaatgtatttagaaaaataaacaaataggggttccgcgcacatttccccgaaaagtgccacctgacgtcgacggatcgggagatcctagcgttt

SEQ ID NO: 5 - protein, 264 aa, Artificial Sequence
  Description of Artificial Sequence Synthetic polypeptide
MDPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 6 - protein, 132 aa, Artificial Sequence
  Description of Artificial Sequence Synthetic polypeptide
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

(19) United States
(12) Patent Application Publication
(10) Pub. No.: US 2009/0053691 A1
(43) Pub. Date: Feb. 26, 2009

(54) Cell Line and Methods for Determining Viral Titer

(75) Inventors: Steven Peppers, Grand Island, NY (US); Steven H. Harwood, Eugene, OR (US); Robert P. Bennett, Encinitas, CA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/176,664

(22) Filed: Jul. 21, 2008

Related U.S. Application Data
(63) Continuation of application No. 11/427,580, filed on Jun. 29, 2006, now abandoned.
(60) Provisional application No. 60/695,804, filed on Jun. 30, 2005.

(51) Int. Cl. C12Q 1/70

(52) U.S. Cl. 435/5

(57) ABSTRACT

The present invention relates to cells, methods, compositions and kits for determining the concentration of virus in a stock, i.e., determining the titer of a viral stock.

### BACKGROUND OF THE INVENTION

1. Field of the Invention

The present invention relates to the fields of biotechnology and molecular biology. In particular, the present invention relates to stably transfected cell lines and methods for using the cell lines to determine the titer of viral stocks.

2. Related Art

Recombinant viruses are currently used in wide variety of applications. Viruses may be used for medical applications, for example, in gene therapy applications and/or as vaccines. Viruses may also be used in biotechnology applications, for example, as vectors to clone nucleic acids of interests and/or to produce proteins. Examples of recombinant viruses that have been used include, but are not limited to, herpes viruses (see, for example, U.S. Pat. No. 5,672,344, issued to Kelly, et al.), pox viruses such as vaccinia virus (see, for example, Moss, et al., 1997, in Current Protocols in Molecular Biology, Chapters 16.15-16.18, John Wiley & Sons), papilloma viruses (see, for example, U.S. Pat. No. 6,342,224, issued to Bruck, et al.), retroviruses (see, for example U.S. Pat. No. 6,300,118, issued to Chavez, et al.), adenoviruses (see, for example, U.S. Pat. No. 6,261,807, issued to Crouzet, et al.), adeno-associated viruses (AAV, see for example, U.S. Pat. No. 5,252,479, issued to Srivastava), and coxsackie viruses (see, for example, U.S. Pat. No. 6,323,024).

Adenoviruses are non-enveloped viruses with a 36 kb DNA genome that encodes more than 30 proteins. At the ends of the genome are inverted terminal repeats (ITRs) of approximately 100-150 base pairs. A sequence of approximately 300 base pairs located next to the 5′-ITR is required for packaging of the genome into the viral capsid. The genome as packaged in the virion has terminal proteins covalently attached to the ends of the linear genome.

The genes encoded by the adenoviral genome are divided into early and late genes depending upon the timing of their expression relative to the replication of the viral DNA. The early genes are expressed from four regions of the adenoviral genome termed E1-E4 and are transcribed prior to onset of DNA replication. Multiple genes are transcribed from each region. Portions of the adenoviral genome may be deleted without affecting the infectivity of the deleted virus. The genes transcribed from regions E1, E2, and E4 are essential for viral replication while those from the E3 region may be deleted without affecting replication. The genes from the essential regions can be supplied in trans to allow the propagation of a defective virus. For example, deletion of the E1 region of the adenoviral genome results in a virus that is replication defective. Viruses deleted in this region are grown on 293 cells that express the viral E1 genes from the genome of the cell.

In addition to permitting the construction of a safer, replication-defective viruses, deletion and complementation in trans of portions of the adenoviral genome and/or deletion of non-essential regions make space in the adenoviral genome for the insertion of heterologous DNA sequences. The packaging of viral DNA into a viral particle is size restricted with an upper limit of approximately 38 kb of DNA. In order to maximize the amount of heterologous DNA that may be inserted and packaged, viruses have been constructed that lack all of the viral genome except the ITRs and packaging sequence (see, U.S. Pat. No. 6,228,646). All of the viral functions necessary for replication and packaging are provided in trans from a defective helper virus that is deleted in the packaging signal.

Recombinant adenoviruses have been used as a gene transfer vectors both in vitro and in vivo. Their principal attractions as a gene transfer vector are their ability to infect a wide variety of cells including dividing and non-dividing cells and their ability to be grown in cell culture to high titers. A number of systems to insert heterologous DNA into the adenoviral genome have been developed. The adenoviral genome has been inserted into a yeast artificial chromosome (YAC, see Ketner, et al., PNAS 91:6186-90, 1994). Mutations may be introduced into the genome by transfecting a mutation-containing plasmid into a yeast cell that contains the adenoviral YAC. Homologous recombination between the YAC and the plasmid introduces the mutation into the adenoviral genome. The adenoviral genome can be removed from the YAC by restriction digest and the genome released by restriction digest is infectious when transfected into host cells. A similar system using two plasmids has been developed in E. coli (see Crouzet, et al., PNAS 94:1414-1419, 1997, and U.S. Pat. No. 6,261,807). In this system, the adenoviral genome is introduced into a inc-P derived replicon. Mutations are introduced by homologous recombination with a plasmid containing a ColE1 origin of replication. The ITRs in the inc-P plasmid are flanked by a restriction site not present in the rest of the viral genome, thus, infectious DNA can be liberated from the plasmid by restriction digest.

Baculoviruses are large, enveloped viruses that infect arthropods. Baculoviral genomes are double-stranded DNA molecules of approximately 130 kilobase pairs (kbp) in length. Baculoviruses have gained widespread use as systems in which to express proteins, particularly proteins from eukaryotic organisms (e.g., mammals), as the insect cells used to culture the virus may more closely mimic the post-translational modifications (e.g., glycosylation, acylation, etc.) of the native organism.

Numerous expression systems utilizing recombinant baculoviruses have been developed. General methods for constructing recombinant baculoviruses for expression of heterologous proteins may be found in Piwnica-Worms, et al., (1997) Expression of Proteins in Insect Cells Using Baculovirus Vectors, in Current Protocols in Molecular Biology, Chapter 16, pp. 16.9.1 to 16.11.12, Ausubel, et al. Eds., John Wiley & Sons, Inc. Other expression systems are known, for example, U.S. Pat. No. 6,255,060, issued to Clark, et al. discloses a baculoviral expression system for expressing nucleotide sequences that include a tag. U.S. Pat. No. 5,244,805, issued to Miller, discloses a baculoviral expression system that utilizes a modified promoter not naturally found in baculoviruses. U.S. Pat. No. 5,169,784, issued to Summers, et al. discloses a baculoviral expression system that utilizes dual promoters (e.g., a baculoviral early promoter and a baculoviral late promoter). U.S. Pat. No. 5,162,222, issued to Guarino, et al. discloses a baculoviral expression system that can be used to create stable cells lines or infectious viruses expressing heterologous proteins from a baculoviral immediate-early promoter (e.g., IEN). U.S. Pat. No. 5,155,037, issued to Summers, et al. discloses a baculoviral expression system that utilizes insect cell secretion signal to improve efficiency of processing and secretion of heterologous genes. U.S. Pat. No. 5,077,214, issued to Guarino, et al. discloses the use of baculoviral early gene promoters to construct stable cell lines expression heterologous genes. U.S. Pat. No. 4,879,239, issued to Smith, et al. discloses a baculoviral expression system that utilizes the baculoviral polyhedrin promoter to control the expression of heterologous genes. International patent application WO 98/44141 discloses the use of baculoviral immediate early promoters ie1 and ie2 linked to a Zeocin antibiotic resistance gene in a selection system in insect cell lines.

Various methods of constructing recombinant baculoviruses have been used. A frequently used method involves transfecting baculoviral DNA and a plasmid containing baculoviral sequences flanking a heterologous sequence. Homologous recombination between the plasmid and the baculoviral genome results in a recombinant baculovirus containing the heterologous sequences. This results in a mixed population of recombinant and non-recombinant viruses. Recombinant baculoviruses may be isolated from non-recombinant by plaque purification. Viruses produced in this fashion may require several rounds of plaque purification to obtain a pure strain. Methods to reduce the background of non-recombinant viruses produced by homologous recombination methods have been developed. For example, a linearized baculoviral genome containing a lethal deletion, BACULOGOLD™, is commercially available from BD Biosciences, San Jose, Calif. The lethal deletion is rescued by homologous recombination with plasmids containing baculoviral sequences from the polyhedrin locus.

Methods utilizing direct insertion of foreign sequences into a baculoviral genome are also known. For example, Peakman, et al. (Nucleic Acids Res 20(3):495-500, 1992) disclose the construction of baculoviruses having a lox site in the genome. Heterologous sequences may be moved into the genome by in vitro site-specific recombination between a plasmid having a lox site and the baculoviral genome in the presence of Cre recombinase. U.S. Pat. No. 5,348,886, issued to Lee, et al. discloses a baculoviral expression system that utilizes a bacmid (a hybrid molecule comprising a baculoviral genome and a prokaryotic origin of replication and selectable marker) containing a recombination site for Tn7 transposon. Prokaryotic cells carrying the bacmid are transformed with a plasmid having a Tn7 recombination site and with a plasmid expressing the activities necessary to catalyze recombination between the Tn7 sites. Heterologous sequences present on the plasmid are introduced into the bacmid by site-specific recombination between the Tn7 sites. The recombinant bacmid may be purified from the prokaryotic host and introduced into insect cells to initiate an infection. Recombinant viruses carrying the heterologous sequence are produced by the cells transfected with the bacmid.

Baculoviral genomes that may be used in the practice of the present invention may be entire genomes or may contain one or more deletions, for example, at the polyhedrin locus. Suitable genomes include those from any virus in the family Baculoviridae. Suitable viral genomes include, but are not limited to, those from occluded baculoviruses (e.g., nuclear polyhedrosis viruses (NPV) such as Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Lymantria Dispar Nuclear Polyhedrosis Virus (LdMNPV), Bombyx mori S Nuclear Polyhedrosis Virus (BmNPV), Heliothis zea SNPV (HzSnpv), and Trichoplusia ni SNPV (TnSnpv) and granulosis viruses (GV) (e.g., Plodia interpunctella granulosis virus (PiGV), Trichoplusia ni granulosis virus (TnGV), Pieris brassicae granulosis virus (PbGV), Artogeia rapae granulosis virus (ArGV), and Cydia pomonella granulosis virus (CpGV)). Suitable genomes also include, but are not limited to, those from non-occluded baculoviruses (NOB) (e.g., Heliothis zea NOB (HzNOB), Oryctes rhinoceros virus), etc.

Regardless of the type of virus used, in order to achieve a productive infection, it is necessary to contact the cells to be infected with a sufficient quantity of virus. For protein expression purposes, the cells are generally contacted with enough virus to ensure a multiplicity of infection (MOI) of greater than one. In order to ensure the proper MOI, the concentration of infectious viral particles in the viral stock (referred to as the viral titer and typically measured in plaque forming units per milliliter i.e., pfu/ml) must be determined.

A number of systems have been developed for determining the presence of virus in sample, viral infectious activity, or other viral properties. For example, cell lines which contain promoters operably connected to a reporter, wherein the promoter is activated if the cells are infected by a particular virus are described in U.S. Pat. Nos. 5,070,012; 5,418,132; 5,591,579; 5,733,720; 5,851,757; 5,910,411; 5,958,676; 5,939,253; 5,945,276; and 6,071,744, the entire disclosures of which are incorporated herein by reference.

The process of determining the viral titer can be time consuming as it is often necessary to conduct plaque assays or limiting dilution assays that can take anywhere from five days to two weeks to complete. In a plaque assay, cells are infected with varying dilutions of the viral stock in order to produce plates having detectable plaques. Plaques are distinct regions of the cell monolayer in which a cluster of cells show evidence of the cytopathic effect (CPE) of the infecting virus. Plaques result from the infection of a single cell with a single virus and replication and spread of the virus to the surrounding cells. Thus, plaque formation requires that a virus infect a single cell, proceed through an entire viral life cycle including release of the progeny virus from the infected cell and then go through a second infection and life cycle in the surrounding cells until CPE can be observed in the surrounding cells. It may be necessary for multiple rounds of virus release in order to produce a plaque of sufficient size to be readily observed. Limiting dilution assays also require a substantial amount of time a labor. Virus stocks are serially diluted and used to infect permissive cells. Usually eight to twelve separate infections must be performed per dilution of a virus and, thus, these assays are usually conducted in 96 well plates. Reading the plates and inference of a viral titer requires careful observation of each well for CPE. Both plaque assays and limiting dilution assays suffer from the fact that identification of CPE is a subjective standard and, therefore, subject to individual to individual variation. Because of the time involved and the variability of both methods, there exists a need in the art for more rapid and accurate methods of determining the titer of a viral stock. This and other needs are met by the present invention.

### BRIEF SUMMARY OF THE INVENTION

The present invention provides materials and methods that may be used to determine the concentration of virus in a composition (e.g., a viral stock) such as a solution. In some embodiments, the invention provides a cell comprising a selected nucleic acid sequence operably linked to a transcriptional regulatory sequence (e.g., a promoter). The regulatory sequence may be selected such that transcription of the selected nucleic acid sequence is modulated (e.g., activated or repressed) by introduction into the cell of a transacting factor, for example, by infection of the cell with a virus containing and/or expressing the transacting factor. In some embodiments, the transcriptional regulatory sequence may be selected such that no transcription or a negligible amount of transcription of the selected nucleic acid sequence occurs in the absence of the transacting factor (i.e., in the absence of a viral infection).

A cell according to the present invention may be any type of cell. In some embodiments, the cell may be susceptible to infection by one or more types of virus. In some embodiments, cells of the invention may be eukaryotic cells, for example, insect cells, mammalian cells, etc. A suitable cell type may be one that is capable of productive infection by a virus of interest. The selection of suitable cell types for any particular virus of interest is within the ability of one of ordinary skill in the art using routine experimentation. Suitable cells include, but are not limited to, primary epithelial cells (e.g., keratinocytes, cervical epithelial cells, bronchial epithelial cells, tracheal epithelial cells, kidney epithelial cells and retinal epithelial cells) and established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, I-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN₂₀₅ cells, McCoy cells, Mouse L cells, Strain 2071 (Mouse L) cells, L-M strain (Mouse L) cells, L-MTK⁻ (Mouse L) cells, NCTC clones 2472 and 2555, SCC-PSA1 cells, Swiss/3T3 cells, Indian muntjac cells, SIRC cells, C₁₁ cells, and Jensen cells, or derivatives thereof).

In some embodiments, the cells of the invention may be insect cells, for example, cells of the invention may be Lepidopteran cells. Examples of suitable cells or cell lines include, but are not limited to those derived from, Lymantria dispar, Helicoverpa zea cells, Heliothis virescens, Mamestra brassicae, Malocosoma dissiria, Leucania separata, Trichoplusia ni, Anticarsia gemmatalis, Spodoptera exigua, Manduca sexta, Choristoneura fumiferana, Spodoptera frugiperda, Bombyx mori, Heliothis zea, or Estigmene acrea. In some embodiments, cells of the invention may be cells derived from Spodoptera frugiperda, for example, Sf9 or Sf21 cells.

In some embodiments, transcriptional regulatory sequences for use in the present invention may be promoters. When the transcriptional regulatory sequence is a promoter, the promoter may be inactive or negligibly active in the cell in the absence of external stimulation (e.g., introduction of a viral infection). In some embodiments, promoters of the invention may be viral promoters. For example, the promoter may be from a virus that is capable of infecting the cell. Optionally, the promoter may require for activity one or more factors (e.g., transacting factors) that are not normally present in the cell. For example, the promoter may require for activity one or more transcription factors that are not normally present in the cell. Such transcription factors may be encoded by a virus and provided by the virus upon viral infection of the cell. Such transcription factors may also be encoded by the cell but not produced by the cell under normal conditions. Such cell-encoded transcription factors may be induced by viral infection of the cell.

In a particular embodiment, a transcriptional regulatory sequence of the invention may be a baculoviral promoter. For example, promoters for use in the invention may be obtained from occluded baculoviruses (e.g., nuclear polyhedrosis viruses (NPV)) such as Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Lymantria Dispar Nuclear Polyhedrosis Virus (LdMNPV), Bombyx mori S Nuclear Polyhedrosis Virus (BmNPV), Heliothis zea SNPV (HzSnpv), and Trichoplusia ni SNPV (TnSnpv) and granulosis viruses (GV) (e.g., Plodia interpunctella granulosis virus (PiGV), Trichoplusia ni granulosis virus (TnGV), Pieris brassicae granulosis virus (PbGV), Artogeia rapae granulosis virus (ArGV), and Cydia pomonella granulosis virus (CpGV)). Promoters for use in the invention may be obtained from non-occluded baculoviruses (NOB) (e.g., Heliothis zea NOB (HzNOB), Oryctes rhinoceros virus), etc.

Suitable promoters for use in the present invention include, but are not limited to baculoviral immediate early (ie), early, late and very late promoters. In particular embodiments, suitable promoters include baculoviral late expression factor 3 (lef-3) promoter and TLP promoter. The sequences of these promoters are provided in Table 1 as SEQ ID NO:1 and SEQ ID NO:2 respectively.

TABLE 1
lef-3 promoter sequence (SEQ ID NO:1)

ccgagaagaaggcggtttgtataaaacccatttttcgaaatggttaacaa
acttgtttagcatttggatcgtttcgtgttcaaacgcgtcgaaaactttt
aaaacgcaattgccgccgggacgcaggcaaattaaaattagctgcgtctc
gcacatgatcaaatcaaagttgagacgttcttgttcgttttcgcgtccat
taacgtcaaccgagccatctgccaacaccagatcgcacgcgttgccacac
ttgatgctaatctcaaatacaacatttttatcaaacacgtcgcctgactt
gtcgggccccgtaatggttgtgaaatttttgcgtttgcgcactgtcggtt
tgtacacgcacaccgagttgtttgtcaacgtgacgccatacgctttgcaa
agcgggttcaacgacatggtatagttggcaaactcgcccggtccgccgca
caaatccaaaaacgtgtcaacgtgtcggcaaacgtgaaactttttgtcga
tctctgatagttttcgccaacatctaggtctgcgcgttgggcgtttgtca
aataattttgagcgagcgcaaaccaccgacttgctgctgaacgtgttcaa
accatctttgagtttatttaatttttgctgcaacatttttactcttcgtg
tcggtcgcaatgtttgtgtcgaaaaagacggccaacacgctcagcaaaac
tatacaaataaagaacaaaaatacgtacgcaatattaacattgaccgttt
gatcgttaaatcggacgggtctgttcagagccgctcttattctctcgttg
tacattgttaaagtttttgtttttaaattgtacacaatcggcgtgttgta
gtcgaaattttcaaaatcggctttttgaaacattgttctgaacgtgttgt
cgagcggcgtgttgctggccacgtttataatcaactccctccacgctaac
gaacggtgctctggcgacacttcgatttcgtcgccattcagtatttgcca
tcggatagattcccacatatcgacaacagcaat
TLP promoter sequence (SEQ ID NO:2)
tgctagcccaattggccactgttgtacgaaataccgtcgtcaacgtgttt
gaatacatgttggcccgtaccgttgggtaaatctatgcatctggagtcgc
cggaacactcgtactggttgtcagagtttctgatccggttgatgcacgtt
atcagttgtgactcgttattattcaaacatttgaaatattgcgtgtcgcc
gatatcggccgttatgtacgtgtgtccggcgccgttaaacgcgcacggat
gcgcttccacgcacgacattaagttgcgatcaaatattttattcgcgggg
cattcgcccaccacgtggcgcccatttacgcactgcataaactggttgac
gagcaaattggagggaaagtatgatagtatatagccgtctggcctgtttt
cacacaattcgttaactttacactggccggtttccgcgtcaaacgtgtaa
ttatctggacattcttcgactgcgtgcgctccgtttgcaaaacacctaag
atagaacgtgggatgatacaagtgcgcgttggtagaataatctttgtcca
agtgttggttcaacaccaacgtgtccagcaaacgctcgtccatgggataa
agaccggcagacttgttgtcgcacggcggcacgggaacacattttagttg
tgcgtaatcaaagttaaaatatgcggggcatttcatggtcacgtcggcct
tgtcgccgctcaaaataaactcgttgggattttcatcatttgctctaacg
cgatcgtgtacgattcgatcaacaggttgaaatttttgatttaagaaatc
aaaaatttcaatccggtcatcatgoacgctttcgtgataggtggaaaggt
cgacggtgttgaaccacgttacaatataagtgttttgcataatatccgac
acgtagcctattacgtcgggtgtgggttcgtctgcgttggtgcgcttcac
atattcagtcatcacttggagccgcttggtgaaagtcgtttcgtcaaatt
caaaataaattgccaaatacattaaagtaaacgctattataagaaaa

In a particular embodiment, a transcriptional regulatory sequence of the invention may be a herpes virus promoter. For example, promoters for use in the invention may be obtained from a herpes virus. Suitable herpes viruses include, but are not limited to, Alphaherpesvirinae such as Simplexviruses (e.g., Human herpesvirus 1), Varicelloviruses (e.g., Human herpesvirus 3 also known as Varicella-zoster virus), Betaherpesvirinae such as Cytomegaloviruses (e.g., Human herpesvirus 5), Muromegaloviruses (e.g., Mouse cytomegalovirus 1), Roseoloviruses (e.g., Human herpesvirus 6), Gammaherpesvirinae such as Lymphocryptoviruses (e.g., Human herpesvirus 4), and Rhadinoviruses (e.g., Ateline herpesvirus 2).

Suitable promoters that may be obtained from a herpesvirus include, but are not limited to, the promoters for the α class gene products. For example, 5 ICP (infected cell proteins) constitute the α-class. They are designated 0, 4, 22, 27, and 47. Promoters for these genes may be used in the practice of the present invention.

In some embodiments, the present invention provides a cell and/or cell line comprising a selected nucleic acid sequence operably linked to a transcriptional regulatory sequence (e.g., a promoter). The cell and/or cell line may be stably transfected with a transcriptional unit comprising a selected nucleic acid sequence operably linked to a transcriptional regulatory sequence (e.g., a promoter). A selected nucleic acid sequence may be any sequence the transcription of which may be detected. For example, in some embodiments, a selected nucleic acid sequence may encode one or more polypeptides. Polypeptides encoded by selected nucleic acid sequences may have one or more characteristics that can be detected. For example, a polypeptide may have one or more enzymatic activity, one or more epitopes and the like. A polypeptide may be directly detectable, for example, may be fluorescent (e.g., green fluorescent protein). In particular embodiments, a polypeptide expressed from the selected nucleic acid sequence may have one or more enzymatic activities. Suitable enzymatic activities include, but are not limited to, β-lactamase activity, β-galactosidase activity, β-glucuronidase activity, luciferase activity, chloramphenicol acetyl transferase activity, etc. In one specific embodiment, a polypeptide expressed from a selected nucleic acid sequence may have β-lactamase activity.

In another aspect, the present invention provides methods of determining the presence or absence of virus in a solution and, if present, determining the concentration of virus in the solution. In a particular aspect, the present invention provides methods of determining the titer of a viral stock. Such methods may entail contacting cells with a sample of the viral stock, wherein the cells comprise a selected nucleic acid sequence operably linked to a transcriptional regulatory sequence. The transcriptional regulatory sequence may be selected so that infection of the cell with a virus results in transcription of the selected nucleic acid sequence. Methods of the invention may also entail identifying cells in which the selected nucleic acid sequence is transcribed.

In some embodiments, cells used in the methods of determining the concentration of virus in a solution may be insect cells. For example, cells suitable for use in this aspect of the present invention may be Lepidopteran cells. Examples of suitable cells include, but are not limited to, Lymantria dispar cells, Helicoverpa zea cells, Heliothis virescens cells, Mamestra brassicae cells, Malocosoma disstria cells, Leucania separata cells, Trichoplusia ni cells, Anticarsia gemmatalis cells, Spodoptera exigua cells, Manduca sexta cells, Choristoneura fumiferana cells, Spodoptera frugiperda cells, Bombyx mori cells, Heliothis zea cells, or Estigmene acrea cells. In some embodiments, cells of the invention may be Spodoptera frugiperda cells, for example, Sf9 or Sf21 cells.

In some methods of determining the concentration of virus in a solution, a transcriptional regulatory sequences may be a viral promoter. For example, the transcriptional regulatory sequence may be a transcriptional regulatory sequence from a virus that infects insect cells. In a particular embodiment, the transcriptional regulatory sequence may be a baculoviral promoter. Examples of suitable baculoviruses from which to obtain a promoter for use in the present invention include, but are not limited to, occluded baculoviruses (e.g., nuclear polyhedrosis viruses (NPV)) such as Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Bombyx mori S Nuclear Polyhedrosis Virus (BmNPV), Heliothis zea SNPV (HzSnpv), and Trichoplusia ni SNPV (TnSnpv) and granulosis viruses (GV) (e.g., Plodia interpunctella granulosis virus (PiGV), Trichoplusia ni granulosis virus (TnGV), Pieris brassicae granulosis virus (PbGV), Artogeia rapae granulosis virus (ArGV), and Cydia pomonella granulosis virus (CpGV)). Promoters for use in the invention may be obtained from non-occluded baculoviruses (NOB) (e.g., Heliothis zea NOB (HzNOB), Oryctes rhinoceros virus), etc. In some embodiments, a transcriptional regulatory sequence may be a temporally regulated viral promoter, for example, a viral early promoter or a viral late promoter.

In some methods of determining the concentration of virus in a solution, a selected nucleic acid sequence may encode a polypeptide and modulation (e.g., activation and/or stimulation) of transcription of the selected nucleic acid sequence (e.g., upon viral infection) may result in the expression of the polypeptide from the transcribed mRNA. In one aspect, a polypeptide expressed from a selected nucleic acid sequence may have one or more characteristics that permit its detection. For example, a polypeptide expressed from a selected nucleic acid sequence may have one or more enzymatic activities that permit detection of the expressed polypeptide. Those skilled in the art will appreciate that virtually any enzymatic activity can be detected and polypeptides having such an activity are within the scope of the present invention. In some particular embodiments, a polypeptide expressed from a selected nucleic acid sequence may have an enzymatic activity selected from the group consisting of β-lactamase activity, β-galactosidase activity, β-glucuronidase activity, and luciferase activity. In some embodiments, polypeptides expressed from selected nucleic acid sequences may have characteristics other than enzymatic activity that permit its detection. In some particular embodiments, a polypeptide expressed from a selected nucleic acid sequence may have one or more detectable spectral qualities (e.g., may be fluorescent such as green fluorescent protein).

In some methods of determining the concentration of virus in a solution, it may be desirable to contact the cells with one or more reagents in order to detect activation and/or stimulation of transcription from the transcriptional regulatory sequence. For example, when a polypeptide having enzymatic activity is expressed as a result of activation and/or stimulation of transcription from the selected nucleic acid sequence, it may be desirable to contact the cells with substrate for the enzymatic activity. In some embodiments, the substrate will undergo a detectable change as a result of the enzymatic activity. For example, the substrate may be one color, have one absorbance spectrum and/or fluorescence emission spectrum prior to reacting with the enzymatic activity and may have a different color, absorbance spectrum, and/or fluorescence emission spectrum after reacting with the enzymatic activity.

In some methods of determining the concentration of virus in a solution, cells may be contacted with a suitable detection reagent without prior processing of the cells. For example, when a cell-permeable substrate is to be used to detect an intracellular enzymatic activity, the cells may be contacted directly with the substrate (e.g., the substrate may be added to the medium in which the cells are growing). In some embodiments, the cells may be processed prior to being contacted with a suitable detection reagent (e.g., a lysate of the cells may be prepared, the cells may be fixed, etc.). In some embodiments, cells may be processed after being contacted with a suitable detection reagent.

In another aspect, the present invention provides a method of monitoring progression of a viral infection in a cell. Methods of this type may entail infecting a cell with a virus. Cells suitable for practicing this aspect of the invention may comprise a selected nucleic acid sequence operably linked to a transcriptional regulatory sequence. Transcriptional regulatory sequences according to this aspect may modulate (e.g., increase or decrease) transcription of the selected nucleic acid sequence when the cell is infected with the virus. Such methods may include quantifying the amount of the selected nucleic acid sequence that is transcribed, the amount of polypeptide that is translated, or the amount of protein activity (e.g., the amount of enzymatic activity) generated. The transcribed nucleic acid may be quantified directly (e.g., as RNA) or indirectly (e.g., as polypeptide translated from the RNA). In some embodiments, a polypeptide having one or more enzymatic activities is encoded by the selected nucleic acid sequence and quantifying comprises determining the amount of enzymatic activity. Further, the invention includes methods where the amount of substrate converted by the enzyme is quantified.

In another aspect, the present invention provides a method of monitoring a viral infection of a cell population. Such a method may entail infecting a cell population with virus, wherein one or more of the cells of the population comprise a selected nucleic acid sequence operably linked to a transcriptional regulatory sequence. A suitable transcriptional regulatory sequence is one that modulates (e.g., increases or decreases) transcription of the selected nucleic acid sequence when the cell is infected with the virus. The method may include obtaining a sample of the infected cell population and quantifying the amount of the selected nucleic acid sequence that is transcribed and/or translated in the sample.

Any cells may be used in the methods of monitoring a viral infection so long as the cells may be infected by the virus of interest. In some embodiments, the cells may be insect cells. For example, cells suitable for use in this aspect of the present invention may be Lepidopteran cells. Examples of suitable cells include, but are not limited to, Lymantria dispar cells, Helicoverpa zea cells, Heliothis virescens cells, Mamestra brassicae cells, Malocosoma disstria cells, Leucania separata cells, Trichoplusia ni cells, Anticarsia gemmatalis cells, Spodoptera exigua cells, Manduca sexta cells, Choristoneura fumiferana cells, Spodoptera frugiperda cells, Bombyx mori cells, Heliothis zea cells, or Estigmene acrea cells. In some embodiments, cells of the invention may be Spodoptera frugiperda cells, for example, Sf9 or Sf21 cells. In some embodiments, a transcriptional regulatory sequence may be a viral promoter, for example, from a virus that infects insect cells. Examples of viruses from which a suitable transcriptional regulatory sequence may be obtained include baculoviruses, for example, occluded viruses (e.g., nuclear polyhedrosis viruses (NPV) such as Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Bombyx mori S Nuclear Polyhedrosis Virus (BmNPV), Heliothis zea SNPV (HzSnpv), and Trichoplusia ni SNPV (TnSnpv)) and granulosis viruses (GV) (e.g., Plodia interpunctella granulosis virus (PiGV), Trichoplusia ni granulosis virus (TnGV), Pieris brassicae granulosis virus (PbGV), Artogeia rapae granulosis virus (ArGV), and Cydia pomonella granulosis virus (CpGV)). Promoters for use in the invention may be obtained from non-occluded baculoviruses (NOB) (e.g., Heliothis zea NOB (HzNOB), Oryctes rhinoceros virus), etc. A promoter for use in the present invention may be a temporally regulated promoter (e.g., a viral early promoter or a viral late promoter). In some embodiments, promoters for use in the present invention include, but are not limited to, the lef-3 promoter and the TLP promoter.

In some methods of monitoring a viral infection, a selected nucleic acid sequence may encode a polypeptide. A polypeptide expressed from the selected nucleic acid sequence may have an enzymatic activity. When a polypeptide having an enzymatic activity is expressed from the selected nucleic acid sequence, quantifying may comprise measuring an amount of enzymatic activity. Examples of enzymatic activities that may be measured include, but are not limited to, β-lactamase activity, β-galactosidase activity, β-glucuronidase activity, luciferase activity. In other embodiments, a polypeptide expressed from the selected nucleic acid sequence may be fluorescent.

In some methods of monitoring a viral infection, identifying cells in which the selected nucleic acid sequence is transcribed may comprise contacting the cells with a substrate for an enzymatic reaction. The cells may directly contacted with the substrate, for example, if the substrate is cell permeable or the cells may be processed before being contacted with an enzymatic substrate (e.g., the cells may be fixed or lysed). In some embodiments, the cells may be contacted with the substrate and then processed, for example, lysed.

In another aspect, the present invention provides methods of producing polypeptides. In some embodiments, methods of this type may entail the use of a cell comprising a selected nucleic acid sequence encoding a polypeptide operably linked to a transcriptional regulatory sequence, wherein the transcriptional regulatory sequence modulates transcription of the selected nucleic acid sequence when a transacting factor is introduced into the cell. Transcription of the selected nucleic acid sequence may be stimulated by introducing the transacting factor into the cell. The cell may then be incubated under conditions causing the expression of the polypeptide. In embodiments of this type, any suitable cells may be used, for example, insect cells, mammalian cells, etc. In some particular embodiments, the cells may be insect cells. For example, cells suitable for use in this aspect of the present invention may be Lepidopteran cells. Examples of suitable cells include, but are not limited to, Lymantria dispar cells, Helicoverpa zea cells, Heliothis virescens cells, Mamestra brassicae cells, Malocosoma disstria cells, Leucania separata cells, Trichoplusia ni cells, Anticarsia gemmatalis cells, Spodoptera exigua cells, Manduca sexta cells, Choristoneura fumiferana cells, Spodoptera frugiperda cells, Bombyx mori cells, Heliothis zea cells, or Estigmene acrea cells. In some embodiments, cells of the invention may be Spodoptera frugiperda cells, for example, Sf9 or Sf21 cells. In one embodiment, cells for use in this aspect of the invention may be Spodoptera frugiperda cells, for example, Sf9 cells or Sf21 cells.

In some methods of producing polypeptides according to the present invention, the transcriptional regulatory sequence may be a viral promoter, for example, a promoter from a virus that infects insect cells. In some particular embodiments, a baculoviral promoter may be used. Suitable sources for baculoviral promoters include, but are not limited to, occluded viruses (e.g., nuclear polyhedrosis viruses (NPV) such as Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Lymantria Dispar Nuclear Polyhedrosis Virus (LdMNPV), Bombyx mori S Nuclear Polyhedrosis Virus (BmNPV), Heliothis zea SNPV (HzSnpv), and Trichoplusia ni SNPV (TnSnpv)) and granulosis viruses (GV) (e.g., Plodia interpunctella granulosis virus (PiGV), Trichoplusia ni granulosis virus (TnGV), Pieris brassicae granulosis virus (PbGV), Artogeia rapae granulosis virus (ArGV), and Cydia pomonella granulosis virus (CpGV)). Promoters for use in the invention may be obtained from non-occluded baculoviruses (NOB) (e.g., Heliothis zea NOB (HzNOB), Oryctes rhinoceros virus), etc. A promoter for use in the present invention may be a temporally regulated promoter (e.g., a viral early promoter or a viral late promoter). In some embodiments, promoters for use in the present invention include, but are not limited to, the lef-3 promoter and the TLP promoter.

Methods of producing polypeptides according to the present invention are particularly suitable to the production of polypeptides that are detectable in the cell. In the presence of the transacting factor, cells containing a selected nucleic acid sequence encoding a detectable polypeptide may be identified.

In any of the above-described methods, a transacting factor may be a polypeptide. A polypeptide transacting factor may be introduced into a cell by transfection, e.g., of the polypeptide or of a nucleic acid encoding the polypeptide. Alternatively, the polypeptide transacting factor may be introduced into a cell by a viral infection. The polypeptide transacting factor may be one normally expressed by the virus or may be one heterologous to the virus. When the polypeptide transacting factor is heterologous to the virus, it may be cloned into the virus such that it is expressed upon viral infection. Suitable transacting factors include, but are not limited to, viral polypeptides, for example, viral transcription factors. An example of a suitable transacting factor is the baculovirus ie-1 protein.

The invention further provides nucleic acid molecules which function as promoter. As an example, the invention provides nucleic acid molecules which comprise a portion of the nucleotide sequence shown in Table 2 or Table 3 operably linked to heterologous nucleic acid, wherein the portion of the nucleotide sequence shown in Table 2 or Table 3 allows for transcription of heterologous nucleic acid when the nucleic acid molecule is introduced into an insect cell. In specific aspect of the invention, the nucleic acid molecules may be isolated. In other aspect, the nucleic acid molecules comprise a vector.

The present invention also encompasses kits for use in practicing one or more of the methods of the invention. Kits of the invention may comprise one or more containers containing one or more cells of the present invention. For example, a kit may comprise a container containing a cell and/or cell line comprising a selected nucleic acid sequence operably linked to a transcriptional regulatory sequence (e.g., a promoter). The cell and/or cell line may be stably transfected with a transcriptional unit comprising a selected nucleic acid sequence operably linked to a transcriptional regulatory sequence (e.g., a promoter). Kits of the invention may comprise one or more containers containing one or more reagents useful in the practice of the present invention. Kits of the invention may comprise containers containing one or more buffers or buffer salts useful for practicing the methods of the invention. A kit of the invention may comprise a container containing a substrate for an enzyme. For example, when a selected nucleic acid sequence encodes a polypeptide having an enzymatic activity, a kit of the invention may comprise one or more substrates useful for detecting the enzymatic activity. A kit of the invention may comprise a reagent useful for introducing molecules into the cells of the invention. For example, a kit may comprise a container containing a transfection reagent suitable for introducing nucleic acid and/or protein molecules into a cell. Suitable transfection reagents include, but are not limited to, positively charged lipids, and mixtures of positively charged and neutral lipids.

Kits of the invention may comprise a container containing a viral stock of known titer. Preferably, the virus in the stock is of the same type as the virus to be used in the methods of the invention (e.g., for determining the concentration of baculovirus in a solution, a baculoviral stock of known titer may be provided). A stock of known titer may be used to construct a calibration curve, for example, amount of enzyme activity plotted as a function of the amount of virus administered to the cells. The calibration curve could then be used to determine the amount of virus in the solution.

Kits of the invention may comprise one or more computer programs that may be used in practicing the methods of the invention. For example, a computer program may be provided that calculates a concentration of virus in a solution, i.e., a viral titer, from results of an enzymatic assay for an enzymatic activity possessed by a polypeptide encoded by a selected nucleic acid sequence of the invention. Such a computer program may be compatible with commercially available equipment, for example, with commercially available microplate readers. When determining the concentration of virus in a solution, various dilutions of a stock of virus of known titer may be applied to cells in different wells in a microplate. Various dilutions of the solution may also be applied to different wells. The infected cells may be contacted with a reagent to determine enzymatic activity in the cells, which may be read by the microplate reader. Programs of the invention may take the output from microplate reader, prepare a calibration curve from the enzymatic activity observed in the cells infected with known amounts of virus and compare this enzymatic activity to the enzymatic activity observed in the cells infected with unknown amounts of virus to determine how much virus was present in the solution.

The present invention further relates to instructions for performing one or more methods of the invention (e.g., titering virus). Such instructions can instruct a user of conditions suitable for performing methods of the invention. Instructions of the invention can be in a tangible form, for example, written instructions (e.g., typed on paper), or can be in an intangible form, for example, accessible via a computer (e.g., over the internet). Also provided is an instruction set that provides, in part, directions for performing one or more method of the invention. Such an instruction set can instruct a user of conditions suitable for, for example, titering virus. Thus, the invention include instructions and instructions sets for performing one or more methods of the invention, as well as methods for performing methods of the invention by following such instructions.

Thus, in certain embodiments, kits of the invention may comprise one or more component selected from the group consisting of: (a) one or more cell line containing nucleic acid which encodes a reporter operably connected to a promoter (e.g., a promoter which is activated in the presence of a virus or a viral expression product), (b) one or more nucleic acids which encode a reporter operably connected to a promoter, (c) enzymatic substrate (e.g., a substrate which may be used to detect an enzymatic activity such as a beta-lactamase activity), (d) one or more computer programs which may be used for data collection and/or analysis, and (e) one or more sets of instructions for using kit components.

Other embodiments of the invention will be apparent to one or ordinary skill in the art in light of what is known in the art, in light of the following drawings and description of the invention, and in light of the claims.

### BRIEF DESCRIPTION OF THE FIGURES

FIG. 1 shows a vector map of the pTLPblaM plasmid. The sequence of this plasmid is set out in Table 3. pLef3-BLM is identical except that the TLP promoter is replaced by the Lef-3 promoter.

FIG. 2 shows fluorescent microscopic examination of infected TLP blaM Titer Cells loaded with CCF2 as a function of virus input (vertical axis) and time of infection (horizontal axis). Twenty microliters of each dilution of virus supernatant were added per well.

FIG. 3A-3B shows the characterization of clonal isolates. (A) shows aggregate titer curves for multiple clones showing similar overall blue/green (B/G) response as a function of input virus. (B) shows induction ratios for clonal isolates.

FIG. 4A-4C shows data related to the optimization of assay parameters. Assay conditions were optimized with respect to cell number, probenicid, and CCF2 concentration. (A) varying numbers of TLP-10 cells were plated in wells of a 96-well plate. Cells were infected with 1.85×10⁶ pfu of wt AcMNPV HTS for 16 hours. Infected and uninfected cells were loaded with 1 μM final concentration of CCF2 and their B/G ratios compared. Optimal amounts of probenicid (B) and CCF2 (C) were determined by infecting 50,000 cells with varying dilutions of wt AcMNPV HTS for 16 hours, and then loading the cells with a 6× loading solutions containing the indicated amounts of each reagent.

FIG. 5 shows the effect of infection time on the Titer Cell Assay. Titer cells were infected for the indicated period of time with serial dilutions of wtAcMNPV HTS. At one hour prior to the indicated infection period, the cells were loaded with CCF2 according to the standard protocol (GENEBLAZER™ Detection Kit Manual, Version B, part number 25-0661), and the plates were read on the fluorescence plate reader.

FIG. 6 shows a comparison of bottom and top-read protocols. Standard curves generated using the bottom and top read protocols are shown. The titers of three samples were estimated using each method. The 0 minutes and 1 minute were analyzed using one set of standard curves (generated with each protocol, variances pooled, CV=8.6% for bottom, 11% for top) each on the same plate, while the 10 minute sample utilized a set of standard curves that are not shown. Although the CV was higher for the top read protocol, the titer values estimated by each method were similar.

FIG. 7 shows the effect on Titer Cell Assay of using cells grown past log phase. Cells that were deliberately grown past log-phase were infected with varying amounts of HTS and compared to corresponding cells maintained in log phase.

FIG. 8 shows a comparison of expected titer vs. observed titer by Titer Cell Assay. The titer of wt AcMNPV was estimated to be 1.85×10⁹ by limiting dilution. Additionally, stocks of MelSfManI 1-24 and GSTManI viruses were estimated by limiting dilution to have titers of 2.7×10⁹ and 9.0×10⁸ pfu/ml, respectively. The wt Ac sample was diluted to 10, 20, 40, 75, and 90%. The titer of each dilution was read using the Titer Cell Assay using the undiluted sample as a standard. The titer of the two recombinant viruses were also estimated (twice each), using the wtAcMNPV virus as a standard. The line was estimated and plotted using the TREND function in Excel.

FIG. 9 shows Titer Cell Assay data. Wild type AcMNPV HTS (1.85×10⁹ pfu/ml) was diluted by the indicated factors. The undiluted and diluted HTS were analyzed. The total time and “bench” time required is noted. The undiluted HTS, which had been titered independently by limiting dilution, was used as a standard for the Titer Cell Line.

FIG. 10 shows the results of an alpha test (i.e., an internal test) of the Titer Cell Assay. Eight participants selected without regard to previous experience, were provided with a plate of Titer Cells, a protocol (Appendix 2), and three tubes labeled standard, #60, and #60A. Each participant followed the protocol. Their results are graphed. Error bars are +/−the 95% confidence interval provided by the model. The average value obtained for each sample with the standard deviation and coefficient of variation (CV) are indicated.

FIG. 11 demonstrates that the Titer Cell Assay detects only active virus. Wild type AcMNPV HTS was treated with UV light for varying amounts of time. Following treatment, the titer of each stock was estimated using the Titer Cell Assay. Error bars represent +/−95% confidence intervals.

FIG. 12A-12B shows expression of recombinant protein from the Titer Cell Line and correlation with titer estimate and cell fluorescence. (A) Fluorescence of cells infected for the indicated amount of time. (B) Anti-V5 western blot for expression of MelSfManI 1-24 protein.

FIG. 13 shows a plate format used in methods described below in Appendix 1.

FIG. 14 shows blue and green channel fields, with exemplary data, described below in Appendix 1.

FIG. 15A-15C shows exemplary scatter plots of data.

FIG. 16 shows an exemplary average B/G ratio field.

FIG. 17 shows exemplary output fields. The curves for the standard and the unknowns and the titers with confidence limits are displayed in the output tab. The first column displays the dilution required to obtain the B/G ratio that is ½ maximal. The second column gives the titer and confidence limits.

FIG. 18 is a schematic representation of a system for providing a product to a party.

FIG. 19 provides a schematic representation of a system for advising a party as to the availability of a product.

FIG. 20 provides the structure of the fluorescent substrate CCF2-AM.

FIG. 21 provides a schematic representation of the hydrolysis of the fluorescent substrates used in some embodiments of the invention.

FIG. 22 shows examples of symmetry-based sigmoid curve regression and averaged Blue/Green responses for standard. FIG. 22A is a plot of the average Blue/Green ratios of replicates generated at various titers of the standard virus preparation. To derive a sigmoid shaped curve from this data, it is first divided into an Upper Domain (FIG. 22C) and Lower Domain (FIG. 22B), depending on whether the data points lie above or below the half-maximal response. FIG. 22B—the lower domain responses are considered to be linearly correlated when plotted on linear coordinates. The regression equation is determined by linear regression, which gives a lower domain curve. FIG. 22C—the upper domain responses are first transformed into coordinates that effectively “rotate” them 180° so that they resemble a plot of lower domain data. Next, the antilog of the transformed x-coordinate is then used for linear regression. Reversing the transformation steps then gives an upper domain curve. FIG. 22D shows matching up domains and confidence intervals of standard. The curve is adjusted to meet at ED50. Using the regression equations for the upper domain and lower domains, the two values for ED50 are calculated and then the average ED50 determine. The slopes of the two regression lines are adjusted to intersect at a common point, the average ED50. (Curve=adjusted lower domain for Y<½ Max and adjusted upper domain for Y>½ Max) To estimate the confidence interval for ED50_{STD}, for each of the 6 sets of individual titrations of the standard, ED50 of the lower domain data is determined. The standard deviation of the set of ED50 values is calculated and then is divided by the average ED50 to obtain the CV of ED50_{STD}. The 95% confidence interval for the ED50_{STD} appropriate for the CV and number of replicates is determined. FIG. 22E shows the original titer of samples and the confidence intervals. In theory, EC50_{STD} is equal to EC50_{SPL} with respect to the true concentration of virus exposed to the cells. Therefore, the original titer of the sample can be calculated as the EC50_{STD} divided by the dilution level of the sample at the ED50_{SPL}. In this example, Titer SPL=2.472×10⁷/0.0156803=1.577×10⁹ PFU. The 95% confidence interval for the ED50_{SPL} is derived using a pooled coefficient of variance (CV_{ρ}) from ED50_{STD} and ED50_{SPL}, assuming equality of CVs:

CV_{pSplA}={[(CV_{Std}²)*(n_{Std}−1)+(CV_{SplA}²)*(n_{SplA}−1)]/(n_{Std}+n_{SplA}−2)}^{0.5}
Since the original titer of a sample depends upon the division of two independent variables, the 95% confidence interval for the original titer involves a summation of the proportional variances:

CI_{OrigSplA}=Conc_{OrigSpla}*[(CI_{ED50Std}/ED50_{Std})+(CI_{ED50SplA}/ED50_{SplA})²]^{0.5}

### DETAILED DESCRIPTION OF THE INVENTION

### Definitions

In the description that follows, a number of terms used in recombinant nucleic acid technology are utilized extensively. In order to provide a clear and more consistent understanding of the specification and claims, including the scope to be given such terms, the following definitions are provided.

Gene: As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

Homologous Recombination: As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule will therefore have a nucleotide sequence that facilitates the exchange of nucleotide strands between the first nucleic acid molecule and a defined position of the second nucleic acid molecule. Thus, the first nucleic acid will generally have a nucleotide sequence that is sufficiently complementary to a portion of the second nucleic acid molecule to promote nucleotide base pairing.

Homologous recombination requires homologous sequences in the two recombining partner nucleic acids but does not require any specific sequences. In contrast, site-specific recombination that occurs, for example, at recombination sites such as att sites, is not considered to be “homologous recombination,” as the phrase is used herein.

Isolated: As used herein, the term “isolated”, when used to described nucleic acids and polypeptides, means that the molecule referred to is in a form other than that in which it exists in nature. In general, an isolated nucleic acid, for example, can be any nucleic acid that is not part of a genome in a cell, or is separated physically from a cell that normally contains the nucleic acid. It should be recognized that various compositions of the invention comprise a mixture of isolated nucleic acids. As such, it will be understood that the term “isolated” only is used in respect to the isolation of the molecule from its natural state, but does not indicate that the molecule is an only constituent.

Host: As used herein, the term “host” refers to any prokaryotic or eukaryotic (e.g. mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75 m M trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Oligonucleotide: As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

Polypeptide: As used herein, the term “polypeptide” refers to a sequence of contiguous amino acids of any length. The terms “peptide,” “oligopeptide,” or “protein” may be used interchangeably herein with the term “polypeptide.”

Promoter: As used herein, a promoter is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

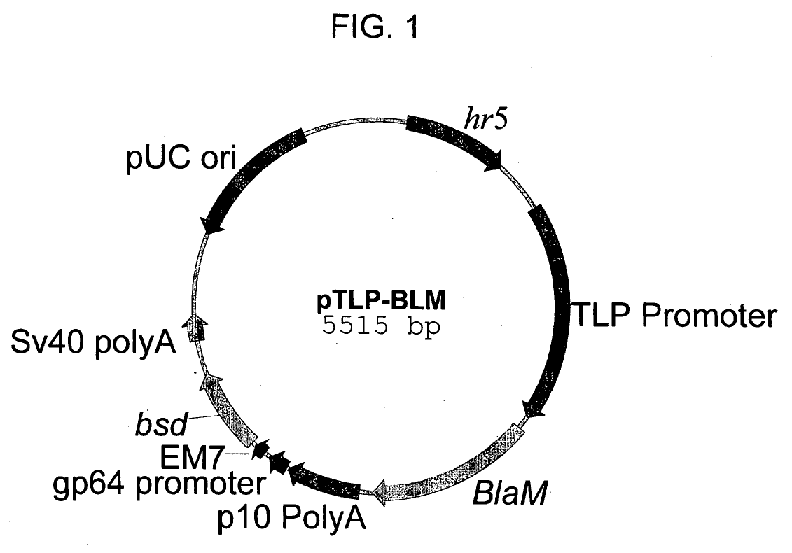

Recognition Sequence: As used herein, the phrase “recognition sequence” or “recognition site” refers to a particular sequence to which a protein, chemical compound, DNA, or RNA molecule (e.g., restriction endonuclease, a modification methylase, topoisomerase, or a recombinase) recognizes and binds. In some embodiments, a recognition sequence may refer to a recombination site, a topoisomerases site, and/or a restriction enzyme site. For example, the recognition sequence for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other examples of recognition sequences are the attB, attP, attL, and attR sequences, which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis) (see Landy, Current Opinion in Biotechnology 3:699-707 (1993)). Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way.

Recombination Site: A used herein, the phrase “recombination site” refers to a recognition sequence on a nucleic acid molecule that participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxp, which is a 34 base pair sequence comprised of two 13 base pair inverted repeats (serving as the recombinase binding sites) flanking an 8 base pair core sequence (see FIG. 1 of Sauer, B., Curr. Opin. Biotech 5:521-527 (1994)). Other examples of recombination sites include the attB, attP, attL, and attR sequences described in U.S. provisional patent applications 60/136,744, filed May 28, 1999, and 60/188,000, filed Mar. 9, 2000, and in co-pending U.S. patent application Ser. Nos. 09/517,466 and 09/732,91—all of which are specifically incorporated herein by reference—and mutants, fragments, variants and derivatives thereof, which are recognized by the recombination protein λ Int and by the auxiliary proteins integration host factor (IHF), FIS and excisionase (Xis) (see Landy, Curr. Opin. Biotech. 3:699-707 (1993)).

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site flanked by recombination sites.

Selected Nucleic Acid Sequence: A used herein, the phrase “selected nucleic acid sequence” encompasses any nucleic acid sequence of interest. A selected nucleic acid sequence may encode a polypeptide. Polypeptides encoded by selected nucleic acid sequences may possesses one or more detectable characteristics. Detectable characteristics are any property that can be directly and/or indirectly determined. Suitable detectable characteristics include, but are not limited to, enzymatic activities and spectral characteristics (e.g., absorbance and/or fluorescence).

Structural Gene: As used herein, the phrase “structural gene” refers to refers to a nucleic acid that is transcribed into messenger RNA that is then translated into a sequence of amino acids characteristic of a specific polypeptide.

Topoisomerase recognition site. As used herein, the term “topoisomerase recognition site” or “topoisomerase site” means a defined nucleotide sequence that is recognized and bound by a site specific topoisomerase. For example, the nucleotide sequence 5′-(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; PCT/US98/12372). In comparison, the nucleotide sequence 5′-GCAACTT-3′ is the topoisomerase recognition site for type IA E. coli topoisomerase III.

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of a selected nucleic acid sequence into messenger RNA or into untranslated RNA. Examples of transcriptional regulatory sequences include, but are not limited to, promoters, enhancers, repressors, operators (e.g., the tet operator), and the like.

Vector: As used herein, the term “vector” refers to a nucleic acid molecule (preferably DNA) that provides a useful biological or biochemical property to an insert. A vector may be a nucleic acid molecule comprising a transcriptional regulatory sequence and/or a selected nucleic acid sequence. Examples of vectors include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more recognition sites (e.g., two, three, four, five, seven, ten, etc. recombination sites, restriction sites, and/or topoisomerases sites) at which the sequences can be manipulated in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Other terms used in the fields of recombinant nucleic acid technology and molecular and cell biology as used herein will be generally understood by one of ordinary skill in the applicable arts.

### Overview

The present invention relates to cells, methods, compositions and kits for determining the concentration of virus in a stock, i.e., determining the titer of a viral stock. The present invention also provides methods of monitoring the progress of a viral infection in a single cell and in a population of cells. In some embodiments, the present invention provides materials and methods for the production of polypeptides in cells, in particular, for the production of polypeptides that are toxic to the cells. The present invention also provides kits useful for constructing cells of the invention and for carrying out the methods of the invention.

### Transcriptional Regulatory Sequences

Transcriptional regulatory sequences of the invention may be of any type known to those of skill in the art. In some embodiments, a transcriptional regulatory sequence of the invention is selected such that it responds in a detectable fashion to the presence of one or more transacting factors that are not normally present in the particular cell type to be used. For example, a transcriptional regulatory sequence of the invention may be inactive or negligibly active in a particular cell type in the absence of one or more transacting factors that are not normally present in the particular cell type to be used. In embodiments of this type, upon introduction of the transacting factor, the transcriptional regulatory sequence of the invention is stimulated and transcription of an operably linked selected nucleic acid sequence occurs. Alternatively, a transcriptional regulatory sequence of the invention may be constitutively active in the absence of a transacting factor not normally present in the particular cell type to be used. Upon introduction of the transacting factor, the transcriptional regulatory sequence may be repressed and transcription of the selected nucleic acid sequence decreased or prevented. As long as the change in transcription level of the selected nucleic acid sequence modulated by the interaction of the transcriptional regulatory sequence and the transacting factor is sufficient to be readily detectable, the transcriptional regulatory sequence may be used in the methods of the present invention.

Transcriptional regulatory sequences suitable for use in the present invention include promoters. As discussed above, promoters may be inactive or negligibly active in a selected cell type or may be constitutively active. Promoters for use in the invention may be viral promoters, for example, baculoviral promoters.

Promoters suitable for use in the present invention also include those with insertions, deletions or substitutions of one, two, three, four, or more nucleotide bases within the native promoter sequence that are at least 50% identical, at least 55% identical, at least 60% identical, at least 65% identical, at least 70% identical, at least 75% identical, at least 80% identical, at least 85% identical, at least 90% identical, or at least 95% identical to the native promoter so long as the modified promoter retains its activity and its responsiveness to the transacting factor.

As a practical matter, whether any particular nucleic acid molecule is at least 50%, 60%, 70%, 75%, 80%, 85%, 90%, 95%, 96%, 97%, 98% or 99% identical to, for instance, a given promoter sequence or portion thereof can be determined conventionally using known computer programs such as DNAsis software (Hitachi Software, San Brurio, Calif.) for initial sequence alignment followed by ESEE version 3.0 DNA/protein sequence software (cabot@trog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711); which employs a local homology algorithm (Smith and Waterman, Advances in Applied Mathematics 2: 482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed. Computer programs such as those discussed above may also be used to determine percent identity and homology between two proteins at the amino acid level.

### Selected Nucleic Acid Sequence

Selected nucleic acid sequences for use in the present invention may be any sequence the transcription of which can be detected. In some embodiments, selected nucleic acid sequences according to the present invention may encode a polypeptide. In such cases, transcription of the selected nucleic acid sequence may be detected by detecting the presence of the polypeptide.

A polypeptide encoded by a selected nucleic acid sequence of the invention may have one or more characteristics that are detectable. For example, a polypeptide may have an enzymatic activity that is detectable. Examples of polypeptides having an enzymatic activity include, but are not limited to, β-galactosidase, β-lactamase, β-glucuronidase, chloramphenicol acetyl transferase and any other enzymatic activity known to those skilled in the art. Other suitable polypeptides having an enzymatic activity are known to those skilled in the art and are within the scope of the present invention.

A polypeptide encoded by a selected nucleic acid sequence of the invention may have a spectral property that makes it readily detectable. For example, a polypeptide encoded by a selected nucleic acid sequence of the invention may be colored or absorb a particular frequency of light. In some embodiments, a polypeptide encoded by a selected nucleic acid sequence of the invention may be a fluorescent protein, for example, green fluorescent protein (GFP), yellow fluorescent protein (YFP), red fluorescent protein (RFP), cyan fluorescent protein (CFP).

A polypeptide encoded by a selected nucleic acid sequence of the invention may be a cell surface protein. Upon transcription and translation of the selected nucleic acid sequence, the polypeptide is expressed on the surface of the cell. The presence of the polypeptide may be detected using standard methodology, for example, one or more epitopes on the polypeptide may be detected using antibodies specific to the epitopes. The cell surface polypeptide may also possess an enzymatic activity. The cell may be incubated with a substrate for the enzymatic activity and the activity detected.

### Host Cells

The invention also relates to host cells comprising one or more selected nucleic acid sequences that may be operably linked to one or more transcriptional regulatory sequences of the invention. Host cells may be stably transformed with nucleic acid molecules comprising one or more selected nucleic acid sequences that may be operably linked to one or more transcriptional regulatory sequences. Such nucleic acid molecules may be integrated into the chromosome of the host cell, for example, by homologous recombination, or may be maintained episomally. Techniques for creating stable cell lines are known to those skilled in the art.

Cells of the invention preferably comprise transcriptional regulatory sequences and/or selected nucleic acid sequences of the invention, which may be part of larger nucleic acid molecules. Such larger nucleic acid molecules (e.g., vectors) to be used in the present invention may comprise one or more origins of replication (QRIs), and/or one or more selectable markers. In some embodiments, nucleic acid molecules may comprise two or more ORIs at least two of which are capable of functioning in different organisms (e.g., one in prokaryotes and one in eukaryotes). For example, a nucleic acid may have an ORI that functions in one or more prokaryotes (e.g., E. coli, Bacillus, etc.) and another that functions in one or more eukaryotes (e.g., yeast, insect, mammalian cells, etc.). Selectable markers may likewise be included in nucleic acid molecules of the invention to allow selection in different organisms. For example, a nucleic acid molecule may comprise multiple selectable markers, one or more of which functions in prokaryotes and one or more of which functions in eukaryotes.

Nucleic acid molecules comprising transcriptional regulatory sequences, selected nucleic acid sequences, and/or encoding transacting factors of the invention may be introduced into cells using standard techniques. Methods for introducing nucleic acids molecules of the invention into the host cells described herein, to produce cells of the invention comprising a selected nucleic acid sequence operably linked to a transcriptional regulatory sequence (e.g., a promoter), will be familiar to those of ordinary skill in the art. For instance, the nucleic acid molecules and/or vectors of the invention may be introduced into host cells using well known techniques of infection, transduction, electroporation, transfection, and transformation. The nucleic acid molecules and/or vectors of the invention may be introduced alone or in conjunction with other nucleic acid molecules and/or vectors and/or proteins, peptides or RNAs. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

Representative host cells that may be used according to this aspect of the invention include, but are not limited to, bacterial cells, yeast cells, plant cells and animal cells. Preferred animal host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly NIH3T3; 293, CHO, COS, VERO, BHK and human cells). Preferred yeast host cells include Saccharomyces cerevisiae cells and Pichia pastoris cells. These and other suitable host cells are available commercially, for example, from Invitrogen Corporation, (Carlsbad, Calif.), American Type Culture Collection (Manassas, Va.), and Agricultural Research Culture Collection (NRRL; Peoria, Ill.).

### Detecting Transcription of the Selected Nucleic Acid Sequence

The transcription of the selected nucleic acid sequence may be detected using any technique known to those of skill in the art. The transcription may be detected directly, for example, by detecting the mRNA, for example, by RT-PCR. The transcription may be detected indirectly, for example, by measuring one or more characteristics of a polypeptide encoded by a selected nucleic acid sequence of the invention.

Transcription and translation of a polypeptide-encoding selected nucleic acid sequence may make it possible to identify host cells containing or not containing the selected nucleic acid sequence selection of appropriate conditions. In one aspect, transcription and translation of a polypeptide-encoding selected nucleic acid sequence may enable visual screening of host cells to determine the presence or absence of the selected nucleic acid sequence. For example, a polypeptide encoded by a selected nucleic acid sequence may alter the color and/or fluorescence characteristics of a cell containing it. This alteration may occur in the presence of one or more compounds, for example, as a result of an interaction between a polypeptide encoded by the selectable sequence and the compound (e.g., an enzymatic reaction using the compound as a substrate). Such alterations in visual characteristics can be used to physically separate the cells containing the selectable sequence from those not contain it by, for example, fluorescent activated cell sorting (FACS).

In a specific embodiment of the invention, a selected nucleic acid sequence may encode a polypeptide having an enzymatic activity (e.g., β-lactamase activity) and transcription of the selected nucleic acid sequence may be determined by assaying for the enzymatic activity.

In a specific embodiment of the invention, a selected nucleic acid sequence may be a nucleic acid sequence encoding a polypeptide having β-lactamase activity. Assays for β-lactamase activity are known in the art. U.S. Pat. Nos. 5,955,604, issued to Tsien, et al. Sep. 21, 1999, 5,741,657 issued to Tsien, et al., Apr. 21, 1998, 6,031,094, issued to Tsien, et al., Feb. 29, 2000, 6,291,162, issued to Tsien, et al., Sep. 18, 2001, and 6,472,205, issued to Tsien, et al. Oct. 29, 2002, disclose the use of β-lactamase as a reporter gene and fluorogenic substrates for use in detecting β-lactamase activity and are specifically incorporated herein by reference. In one embodiment of the invention, a selected nucleic acid sequence may be a nucleic acid sequence encoding a polypeptide having β-lactamase activity and transcription from a selected nucleic acid sequence may be identified by assaying the host cells for β-lactamase activity.

A β-lactamase catalyzes the hydrolysis of a β-lactam ring. Those skilled in the art will appreciate that the sequences of a number of polypeptides having β-lactamase activity are known. In addition to the specific β-lactamases disclosed in the Tsien, et al. patents listed above, any polypeptide having β-lactamase activity is suitable for use in the present invention.

β-lactamases are classified based on amino acid and nucleotide sequence (Ambler, R. P., Phil. Trans. R. Soc. Lond. [Ser.B.] 289: 321-331 (1980)) into classes A-D. Class A β-lactamases possess a serine in the active site and have an approximate weight of 29 kd. This class contains the plasmid-mediated TEM β-lactamases such as the RTEM enzyme of pBR322. Class B β-lactamases have an active-site zinc bound to a cysteine residue. Class C enzymes have an active site serine and a molecular weight of approximately 39 kd, but have no amino acid homology to the class A enzymes. Class D enzymes also contain an active site serine. Representative examples of each class are provided below with the accession number at which the sequence of the enzyme may be obtained in the indicated database.

| | |
|---|---|
| Accession | |
| No. | Database |

Class A β-lactamases

| | | |
|---|---|---|
| Bacteroides fragilis CS30 | L13472 | GenBank |
| Bacteroides uniformis WAL-7088 | P30898 | SWISS-PROT |
| PER-1, P. aeruginosa RNL-1 | P37321 | SWISS-PROT |
| Bacteroides vulgatus CLA341 | P30899 | SWISS-PROT |
| OHIO-1, Enterobacter cloacae | P18251 | SWISS-PROT |
| SHV-1, K. pneumoniae | P23982 | SWISS-PROT |
| LEN-1, K. pneumoniae LEN-1 | P05192 | SWISS-PROT |
| TEM-1, E. coli | P00810 | SWISS-PROT |
| Proteus mirabilis GN179 | P30897 | SWISS-PROT |
| PSE-4, P. aeruginosa Dalgleish | P16897 | SWISS-PROT |
| Rhodopseudomonas capsulatus SP108 | P14171 | SWISS-PROT |
| NMC, E. cloacae NOR-1 | P52663 | SWISS-PROT |
| Sme-1, Serratia marcescens S6 | P52682 | SWISS-PROT |
| OXY-2, Klebsiella oxytoca D488 | P23954 | SWISS-PROT |
| K. oxytoca E23004/SL781/SL7811 | P22391 | SWISS-PROT |
| S. typhimurium CAS-5 | X92507 | GenBank |
| MEN-1, E. coli MEN | P28585 | SWISS-PROT |
| Serratia fonticola CUV | P80545 | SWISS-PROT |
| Citrobacter diversus ULA27 | P22390 | SWISS-PROT |
| Proteus vulgaris 5E78-1 | P52664 | SWISS-PROT |
| Burkholderia cepacia 249 | U85041 | GenBank |
| Yersinia enterocolitica | Q01166 | SWISS-PROT |
| serotype O:3/Y-56 | | |
| M. tuberculosis H37RV | Q10670 | SWISS-PROT |
| S. clavuligerus NRRL 3585 | Z54190 | GenBank |
| III, Bacillus cereus 569/H | P06548 | SWISS-PROT |
| B. licheniformis 749/C | P00808 | SWISS-PROT |
| I, Bacillus mycoides NI10R | P28018 | SWISS-PROT |
| I, B. cereus 569/H/9 | P00809 | SWISS-PROT |
| I, B. cereus 5/B | P10424 | SWISS-PROT |
| B. subtilis 168/6GM | P39824 | SWISS-PROT |
| 2, Streptomyces cacaoi DSM40057 | P14560 | SWISS-PROT |
| Streptomyces badius DSM40139 | P35391 | SWISS-PROT |
| Actinomadura sp. strain R39 | X53650 | GenBank |
| Nocardia lactamdurans LC411 | Q06316 | SWISS-PROT |
| S. cacaoi KCC S0352 | Q03680 | SWISS-PROT |
| ROB-1, H. influenzae F990/LNPB51/ | P33949 | SWISS-PROT |
| serotype A1 | | |
| Streptomyces fradiae DSM40063 | P35392 | SWISS-PROT |
| Streptomyces lavendulae DSM2014 | P35393 | SWISS-PROT |
| Streptomyces albus G | P14559 | SWISS-PROT |
| S. lavendulae KCCS0263 | D12693 | GenBank |
| Streptomyces aureofaciens | P10509 | SWISS-PROT |
| Streptomyces cellulosae KCCS0127 | Q06650 | SWISS-PROT |
| Mycobacterium fortuitum | L25634 | GenBank |
| S. aureus PC1/SK456/NCTC9789 | P00807 | SWISS-PROT |
| BRO-1, Moraxella catarrhalis ATCC | Z54181 | GenBank; |
| 53879 | Q59514 | SWISS-PROT |

Class B β-lactamase

| | | |
|---|---|---|
| II, B. cereus 569/H | P04190 | SWISS-PROT |
| II, Bacillus sp. 170 | P10425 | SWISS-PROT |
| II, B. cereus 5/B/6 | P14488 | SWISS-PROT |
| Chryseobacterium meningosepticum | X96858 | GenBank |
| CCUG4310 | | |
| IMP-1, S. marcescens AK9373/TN9106 | P52699 | SWISS-PROT |
| B. fragilis TAL3636/TAL2480 | P25910 | SWISS-PROT |
| Aeromonas hydrophila AE036 | P26918 | SWISS-PROT |
| L1, Xanthomonas maltophilia IID 1275 | P52700 | SWISS-PROT |

Class C β-lactamase

| | | |
|---|---|---|
| Citrobacter freundii OS60/GN346 | P05193 | SWISS-PROT |
| E. coli K-12/MG1655 | P00811 | SWISS-PROT |
| P99, E. cloacae P99/Q908R/MHN1 | P05364 | SWISS-PROT |
| Y. enterocolitica IP97/serotype O:5B | P45460 | SWISS-PROT |
| Morganella morganii SLM01 | Y10283 | GenBank |
| A. sobria 163a | X80277 | GenBank |
| FOX-3, K. oxytoca 1731 | Y11068 | GenBank |
| K. pneumoniae NU2936 | D13304 | GenBank |
| P. aeruginosa PAO1 | P24735 | SWISS-PROT |
| S. marcescens SR50 | P18539 | SWISS-PROT |
| Psychrobacter immobilis A5 | X83586 | GenBank |

Class D β-lactamases

| | | |
|---|---|---|
| OXA-18, Pseudomonas aeruginosa Mus | U85514 | GenBank |
| OXA-9, Klebsiella pneumoniae | P22070 | SWISS-PROT |
| Aeromonas sobria AER 14 | X80276 | GenBank |
| OXA-1, Escherichia coli K10-35 | P13661 | SWISS-PROT |
| OXA-7, E. coli 7181 | P35695 | SWISS-PROT |
| OXA-11, P. aeruginosa ABD | Q06778 | SWISS-PROT |
| OXA-5, P. aeruginosa 76072601 | Q00982 | SWISS-PROT |
| LCR-1, P. aeruginosa 2293E | Q00983 | SWISS-PROT |
| OXA-2, Salmonella typhimurium type 1A | P05191 | SWISS-PROT |

For additional β-lactamases and a more detailed description of substrate specificities, consult Bush et al. (1995) Antimicrob. Agents Chemother. 39:1211-1233. Those skilled in the art will appreciate that the polypeptides having β-lactamase activity disclosed herein may be altered by for example, mutating, deleting, and/or adding one or more amino acids and may still be used in the practice of the invention so long as the polypeptide retains detectable β-lactamase activity: An example of a suitably altered polypeptide having β-lactamase activity is one from which a signal peptide sequence has been deleted and/or altered such that the polypeptide is retained in the cytosol of prokaryotic and/or eukaryotic cells. The amino acid sequence of one such polypeptide is provided in Table 2. The amino acid sequence of another such polypeptide, as well as a nucleotide sequence which encodes this amino acid sequence is shown in Table 3.

TABLE 2
Amino acid sequence of a β-lactamase enzyme.

```
Met Gly His Pro Glu Thr Leu Val Lys Val Lys Asp Ala Glu Asp Gln
    1 5 10 15
Leu Gly Ala Arg Val Gly Tyr Ile Glu Leu Asp Leu Asn Ser Gly Lys
                          20 25 30
Ile Leu Glu Ser Phe Arg Pro Glu Glu Arg Phe Pro Met Met Ser Thr
                  35 40 45
Phe Lys Val Leu Leu Cys Gly Ala Val Leu Ser Arg Asp Asp Ala Gly
          50 55 60
Gln Glu Gln Leu Gly Arg Arg Ile His Tyr Ser Gln Asn Asp Leu Val
  65 70 75 80
Glu Tyr Ser Pro Val Thr Glu Lys His Leu Thr Asp Gly Met Thr Val
                                  85 90 95
Arg Glu Leu Cys Ser Ala Ala Ile Thr Met Ser Asp Asn Thr Ala Ala
                        100 105 110
Asn Leu Leu Leu Thr Thr Ile Gly Gly Pro Lys Glu Leu Thr Ala Phe
                115 120 125
Leu His Asn Met Gly Asp His Val Thr Arg Leu Asp His Trp Glu Pro
        130 135 140
Glu Leu Asn Glu Ala Ile Pro Asn Asp Glu Arg Asp Thr Thr Met Pro
145 150 155 160
Val Ala Met Ala Thr Thr Leu Arg Lys Leu Leu Thr Gly Glu Leu Leu
                                165 170 175
Thr Leu Ala Ser Arg Gln Gln Leu Ile Asp Trp Met Glu Ala Asp Lys
                        180 185 190
Val Ala Gly Pro Leu Leu Arg Ser Ala Leu Pro Ala Gly Trp Phe Ile
                195 200 205
Ala Asp Lys Ser Gly Ala Gly Glu Arg Gly Ser Arg Gly Ile Ile Ala
        210 215 220
Ala Leu Gly Pro Asp Gly Lys Pro Ser Arg Ile Val Val Ile Tyr Thr
225 230 235 240
Thr Gly Ser Gln Ala Thr Met Asp Glu Arg Asn Arg Gln Ile Ala Glu
                                245 250 255
Ile Gly Ala Ser Leu Ile Lys His Trp
                        260 265
```

Materials and methods of the invention may also comprise one or more fluorescence resonance energy transfer (FRET)-enabled substrates (e.g., CCF2, CCF4, etc.) to facilitate fluorescence detection of β-lactamase reporter activity. In the absence or presence of β-lactamase reporter activity, cells loaded with the CCF2 or CCF4 substrate fluoresce green or blue, respectively. Comparing the ratio of blue to green fluorescence in a population of live cells or in a cell extract prepared from a sample to a negative control provides a means to quantitate gene expression.

In some embodiments, a β-lactamase for use in the present invention may be the product encoded by the ampicillin resistance gene (bla), which is the bacterial enzyme that hydrolyzes penicillins and cephalosporins. The bla gene is present in many cloning vectors and allows ampicillin selection in E. coli. β-lactamase is not found in mammalian cells.

In some embodiments, materials and methods of the invention may use a modified bla gene as a reporter in mammalian cells. One example is a bla gene derived from the E. coli TEM-1 gene present in many cloning vectors (see, Zlokarnik, et al. (1998) Science 279, 84-88), which has been modified in that 72 nucleotides encoding the first 24 amino acids of β-lactamase were deleted from the N-terminal region of the gene. These 24 amino acids comprise the bacterial periplasmic signal sequence, and deleting this region allows cytoplasmic expression of β-lactamase in mammalian cells. The amino acid at position 24 was mutated from His to Asp to create an optimal Kozak sequence for improved translation initiation. The TEM-1 gene also contains 2 mutations (at nucleotide positions 452 and 753) that distinguish it from the bla gene in pBR322 (see, Sutcliffe, J. G. (1978) Proc. Nat. Acad. Sci. USA 75, 3737-3741).

As described in the above-referenced Tsien et al., United States patents, host cells to be assayed may be contacted with a fluorogenic substrate for β-lactamase activity. In the presence of β-lactamase, the substrate is cleaved and the fluorescence emission spectrum of the substrate is altered. As an example, un-cleaved substrate may fluoresce green (i.e., have an emission maxima at approximately 520 nm) when excited with light having a wavelength of 405 nm and the cleaved substrate may fluoresce blue (i.e., have an emission maxima at approximately 447 nm). By determining the ratio of green fluorescence intensity to blue fluorescence intensity it is possible to determine the amount of β-lactamase produced and from that, to calculate what % of the cells express β-lactamase. Kits for conducting fluorescence-based β-lactamase assays are commercially available, for example, from Invitrogen Corporation products 12578-126, 12578-134, 12578-035, 12578-043, 12578-050, and 12578-068.

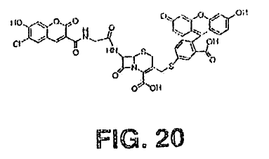

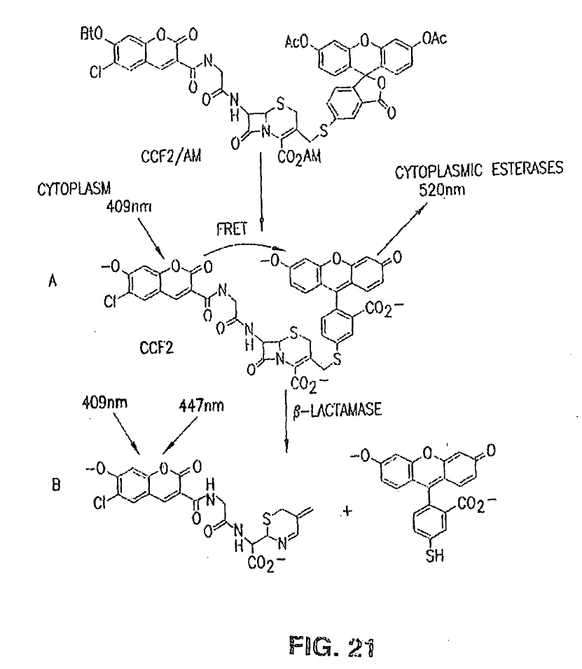

Preferred β-lactam fluorogenic substrates for use in the present invention include those which comprise a fluorescence donor moiety and a fluorescence acceptor moiety linked to a cephalosporin backbone such that, upon hydrolysis of the β-lactam, the acceptor moiety is released from the molecule. Before the β-lactam, is hydrolyzed, the donor and acceptor moiety are positioned such that efficient fluorescence resonance energy transfer (FRET) occurs. Upon excitation with light of a suitable wavelength, fluorescence from the acceptor moiety is observed. After hydrolysis of the β-lactam, the acceptor moiety is released from the molecule and the FRET is disrupted resulting in a change in the fluorescence emission spectrum. An example of a suitable fluorescence donor molecule is a coumarin or derivative thereof (e.g., 6-chloro-7-hydroxycoumarin) and examples of suitable acceptor moieties include, but are not limited to, fluorescein, rhodol, or rhodamine or derivatives thereof. Examples of suitable substrates include CCF2 and the acetoxymethyl ester derivative thereof (CCF2/AM) and CCF4 and the acetoxymethyl ester derivative thereof (CCF4/AM). Those skilled in the art will appreciate that the ester derivatives are membrane permeable and are de-esterified inside a cell by the action of endogenous esterase enzymes. The structure of CCF2 is shown in FIG. 20. A schematic showing entry of the esterified substrate into a host cell, subsequent de-esterification and hydrolysis of CCF2 by a β-lactamase is shown in FIG. 21. CCF2 and CCF4 substrates are described in U.S. Appl. No. 60/511,634, filed, Oct. 17, 2003, the entire disclosure of which is incorporated herein by reference.

Assays for other enzymatic activities are known in the art. Preferred assays for use in the present invention include, but are not limited to, assays that produce colored or fluorescent products and that can be adapted for use in a microplate reader. Assays for β-galactosidase and luciferase are commercially available from, for example, Applied Biosystems, Foster City, Calif., under catalog numbers T1006 and T1035, respectively. Assays for chloramphenicol acetyl transferase are commercially available from, for example, Serologicals Corporation, Norcross, Ga., under catalog number 9359-36. Assays for β-glucuronidase are commercially available, for example, from Bio-Rad, Hercules, Calif., under catalog number 170-3151.

A map of an exemplary vector of the invention is shown in FIG. 1. The nucleotide sequence and various other features of this vector are shown in Table 3.

### Methods of Determining Viral Titer

The present invention provides methods of determining the concentration of a virus in a solution (i.e., determining the titer of a virus). Cells of the invention comprising one or more selected nucleic acid sequences that may be operably linked to one or more transcriptional regulatory sequences may be cultured using standard techniques. To determine a titer of a solution, cells of the invention are contacted with various dilutions of the solution and transcription of the selected nucleic acid sequence is determined. In a preferred embodiment, transcription is determined by assaying an enzymatic activity of a polypeptide encoded by the selected nucleic acid sequence. Any enzymatic assay discussed above as well as any others known to those skilled in the art may be used. The transcriptional regulatory sequence may be a sequence that is activated by the virus. In a particular embodiment, the transcriptional regulatory sequence may be selected from a group consisting of the lef-3 sequence and the TLP sequence disclosed in Table 1 and the selected nucleic acid sequence may encode an enzymatic activity (e.g., β-lactamase).

It may be desirable to contact cells of the invention with a known amount of a suitable virus in order to prepare a standard curve. A suitable virus is one that induces transcription of the selected nucleic acid sequence.

After cells are contacted with virus, they may be grown for a suitable time in order to allow transcription of the selected nucleic acid sequence to occur. Suitable times may be from about 1 hour to about 5 days, from about 1 hour to about 4 days, from about 1 hour to about 3 days, from about 1 hour to about 2 days, from about 1 hour to about 24 hours, from about 1 hour to about 20 hours, from about 1 hour to about 16 hours from about 1 hour to about 12 hours, from about 1 hour to about 8 hours, from about 1 hour to about 7 hours, from about 1 hour to about 6 hours, from about 1 hour to about 5 hours, from about 1 hour to about 4 hours, from about 1 hour to about 3 hours, or from about 1 hour to about 2 hours.

After virus-infected cells are incubated for a sufficient period of time, transcription of the selected nucleic acid sequence may be determined. For example, when the transcription is to be determined by assaying an enzymatic activity encoded by the selected nucleic acid sequence, the virus-infected cells may be contacted with a suitable enzymatic substrate. After contacting with a substrate, the cells may be directly assayed for conversion of the substrate into product by the enzymatic assay. For example, when a cell permeable fluorogenic substrate is used, fluorescence of the whole cells may be determined. Alternatively, the cells may be processed prior to determining the enzymatic activity. For example, the cells may be lysed using standard techniques and all or a portion of the cell lysate may be assayed for enzymatic activity.

In an embodiment, cells of the invention may be plated in a multiwell plate (e.g., a 96 well plate). Typically, each well may receive from about 1×10⁴ to about 1×10⁵ cells, for example, about 5×10⁴ cells per well. Some of the wells may be contacted with a dilution of the solution of unknown virus concentration and some of the wells may be contacted with dilutions of a solution with known virus concentration. After incubation, the cell culture medium may be removed and the cells may be rinsed with a buffer solution and lysed by contacting them with a buffered solution containing a surfactant. A suitable solution is 100 mM potassium phosphate (pH 7.8) containing 0.2% Triton X-100. The pH and the concentration of surfactant should be selected so as not to substantially inhibit or degrade the enzymatic activity to be assayed. After lysis, the lysate may be contacted with a suitable chromogenic or fluorogenic substrate and any other reagents necessary to conduct the enzymatic assay (e.g., salts, divalent metal ions etc.) and the lysate may be incubated a suitable time period to allow the color or fluorescence to be produced. After incubation, the amount of enzymatic activity can be determined using microplate reader.

In particular, methods of the invention include those where a cell line which contains nucleic acid which results in a detectable phenotype upon expression is contacted with a sample containing a virus. Typically, the nucleic acid which results in a detectable phenotype upon expression will be operably connected to a transcription regulatory sequence which is activated in the present of viral nucleic acid or viral expression products. After a certain period of time (e.g., 1 hour, 2 hours 3 hours, 4 hours, 5 hours, 6 hours, etc), the cell line is tested for the presence of the detectable phenotype.

The detectable phenotype may be detected by any number of methods, including visual expression or scanning (e.g., using an automated scanner).

### Methods of Monitoring Progress of a Viral Infection

In one aspect, the present invention provides a method of monitoring the progress of a viral infection in a single cell as well as in a population of cells. Cells of the invention may be infected with virus and then transcription of the selected nucleic acid sequence determined to determine the progress of the infection. For example, to monitor the progress of the infection of a single cell, a cell may be infected with a virus and then contacted with a cell permeable chromogenic or fluorogenic substrate for an enzymatic activity encoded by the selected nucleic acid sequence. As the infection progresses, more of the enzymatic activity is produced resulting in more conversion and/or an increased rate of conversion of the substrate into product. This results in an increase in color or fluorescence in the individual cell.

Methods of this type may be used to monitor the progression of an infection of a large population of cells, for example, in a bioreactor. A virus may be constructed so as to express a polypeptide of interest. The virus may be used to infect cells of the invention in order to produce the polypeptide of interest. After cells in the population are infected with virus, aliquots of the cells can be removed at various time points and the amount of an enzymatic activity encoded by a selected nucleic acid sequence can be determined. This allows one skilled in the art to adjust the incubation period of the infected cells so as to maximize the expression of the polypeptide of interest. This is particularly useful in instances where it is not possible to easily determine directly the amount of the polypeptide of interest in the cells. Methods of this type make it relatively easy to standardize incubation conditions, giving consistent results from bioreactor processes (e.g., consistent protein production, consistent virus production, etc.).

Methods of this type may be used to monitor the progression of an infection while simultaneously expressing a heterologous polypeptide of interest, for example, from a baculovirus. A virus may be constructed so as to express a polypeptide interest. The virus may be used to infect cells of the invention in order to produce the polypeptide of interest. After a population of cells (e.g., cells of the invention) is infected with virus, aliquots of the cells can be removed at various time points and the amount of an enzymatic activity encoded by a selected nucleic acid sequence can be determined. This will allow one skilled in the art to adjust the incubation period of the infected cells so as to maximize the expression of the polypeptide of interest. This will be particularly useful if it is not possible to easily measure the directly the amount of the polypeptide of interest in the cells. Methods of this type will also make it easier to standardize incubation conditions giving more consistent results from bioreactor processes.

### Methods of Expressing a Polypeptide

In some embodiments, the cells of the invention may be used to express a polypeptide of interest. A host cell of the invention may be constructed such that a selected nucleic acid sequence encoding a polypeptide of interest is operably linked to a transcriptional regulatory sequence. The transcriptional regulatory sequence maybe selected such no or a negligible amount of transcription occurs in the absence of one or more transacting factors. When the cell is contacted with the requisite transacting factors, the polypeptide of interest is produced.

Embodiments of this type are well suited for the construction of stable cell lines that are capable of expressing a toxic protein. For example, a selected nucleic acid sequence may encode a protein that is toxic to the host cell. In the absence of transacting factors, the protein is not expressed or is expressed a negligible level (e.g., a level that is not toxic to the cell). This permits large numbers of the cells to be grown. When the cells have grown to a sufficient quantity, the cells may be contacted with the requisite transacting factor or factors (e.g., may be infected with a virus containing or expressing the factors) and production of the polypeptide of interest is induced. The progress of the infection may be monitored as above and the polypeptide of interest may be harvested from the cells and/or the culture medium using standard techniques. Thus, the present invention provides methods for the production and/or expression of proteins that are toxic to cells in which they are produced.

### Data Analysis

The invention also includes data analysis methods which involve detection of a signal generated in a composition (e.g., a sample) in which viral titer is sought to be determined. In many instances, the strength of the signal will be an indication of the amount or concentration of virus in the composition.

The signal may be generated in any number of ways. For example, the signal may result from the production of a protein which has a detectable activity (e.g., is fluorescent, has an enzymatic activity, etc.). One example of such a protein is a β-lactamase. Other examples include green fluorescent protein and cell surface localized proteins which may be detected using, for example, antigen-antibody reactions.

Data analysis methods of the invention may be based upon the detection of a single signal or multiple (e.g., two, three, four, five, etc.) signals. As an example, the β-lactamase substrate CCF2 and/or CCF4 may be used in conjunction with methods for detecting β-lactamase activity. When CCF2 is exposed to excitation light of 405 nm (+/10 nm), this compound emits light in the green portion of the spectrum. When CCF2 is cleaved by a β-lactamase, one of the products emits light in the blue portion of the spectrum. Thus, substrates such as CCF2, for example, may be used in conjunction with nucleic acid encoding a β-lactamase to generate ratio metric emission signals that can employed to determine viral titer. The invention thus includes ratio metric methods for determining viral titer in a sample. Such ratio metric methods may involve two different separate signals where (1) both signals change in intensity or (2) one signal remains constant while the other signal changes intensity.

When ratio metric methods are used to determine viral titer and differences in two different signal intensities are measure in which both signal intensities change, both signal intensities may increase or decrease but at different rates or the intensity of one signal may decrease while the other increases. β-lactamase activity may be measured using a substrate such as CCF2 or CCF4 in methods which employ the latter. More specifically, β-lactamase activity results in a decrease in the amount of fluorescence in the green portion of the spectrum and an increase in the amount of fluorescence in the blue portion of the spectrum. Detection of CCF2 substrate and product may be performed by measuring emissions at 530 nm (+/15 nm) and 460 nm (+/20 nm), respectively. Methods which may be used in conjunction with CCF2 for the detection of α-lactamase activity may be found in the product manuals which accompany Invitrogen Corporation products 12578-126, 12578-134, 12578-035, 12578-043, 12578-050, and 12578-068, the entire disclosures of these manuals are incorporated herein by reference.

In particular systems where the intensity of two signals are measured and the intensity of one signal remains constant, the constant signal may be generated by a protein such as GFP. For example, GFP may be expressed in a cell line which is then used for determining viral titers according to methods of the invention. GFP signal intensity may be used, for example, to establish a baseline signal level for determining the number of cells present in the reaction mixture. Thus, expression of GFP, in this instance, will not correlate with viral infection. The changes in the intensity of the second signal in this system alters in response to viral infection and, typically, will be based upon the detection of signal generated by a molecule other than GFP.

In particular embodiments, a single signal is used in methods of the invention and changes in the intensity of this signal is measured. For example, two copies of nucleic acid encoding GFP may be introduced into a cell line. One copy of the nucleic acid may be operably connected to a promoter which confers expression which is independent of viral infection and the other copy may be operably connected to a promoter which confers expression in the presence of virus. Typically but not always, in such an instance, signal intensity will be measured in two different aliquots of the cell line: one in the absence of virus and the other in the presence of virus. The difference in signal intensity between the aliquots is then analyzed to arrive at the viral titer.

The signal generated by cell lines may be detected on an individual cell basis or in a cell mixtures as a whole. More specifically, the presence or signal, as well as signal intensity, may be measured in individual cells. In such an instance, the number of cells which generate a signal may be compared to the number of cells which do not generate a signal to arrive at the viral titer. Similarly, the number of cells which generate signal of a certain intensity (e.g., over a certain intensity) may be compared to the number of cells which generate a signal of a different intensity (e.g., under a certain intensity) to arrive at the viral titer. In other instances, the signal intensity or signal intensities in the cell population will be measured to arrive at the viral titer.

Method of the invention include those which generate data that can be extrapolated to cover the analysis of any response distribution displaying the appearance of, for example, a sigmoid curve. Such a curve may be generated when a signal response increases in direct proportion to low dosages or concentrations of an agent which results in the production of a signal (e.g., a virus), but as the concentration increases the signal exhibits saturation features, eventually approaching some finite maximal signal. In such instances, when the signal is plotted vs. the log of concentration or dosage, the sigmoid shape of the curve is rather symmetrical about the center (mid-signal). Such data analysis methods are based on linear regressions of normal and transformed sets of data and are not a traditional curve linear regression. As such, no specialized curve-fitting software is require for data interpretation.

In many instances, it is desirable to measure a concentration which corresponds with the mid-signal (the EC50 concentration or ED50 dose of the agent which results in the production of a signal), and the original concentration (or titer) of a test agent in comparison to a standard agent preparation. The test agent will typically be the virus in the composition and the standard agent will typically be a control composition which contains a known amount of the agent. This allows for the generation of confidence limits prescribed about the original titer of the test agent. Other examples of applications of such methods may include ELISA, RIA, cell-substrate fluorescence measurements, enzyme-substrate reaction rates, column-protein binding kinetics, volume-displacement kinetics, etc.

The invention additional includes software and the use of software for interpreting signal intensity data. In many instances, the interpreting of the signal intensity data will result in a determination of the viral titer of a composition.

Along these lines, the invention methods which involve detection of signal intensity followed by interpretation of those data to arrive at a viral titer.

The invention also includes methods wherein multiple compositions for which viral titers are to be determined are analyzed at the same time or in rapid succession. For example, compositions (e.g., samples) may be housed in individual wells of a multiwell plate (e.g., a 96 well plate) and signal intensity may be measured in a plurality of wells simultaneously or one well at a time using, for example, a commercially available fluorescent plate reader.

Once signal intensity data has been collected, it may be analyzed, for example, visually (e.g., by a visual comparison of numbers representing signal intensity, by the manual drawing of a graph) or by computer analysis. Computer analysis may be performed either at the site of where the data is obtained or elsewhere. For example, signal intensity data may be (1) entered into the appropriate window or windows in, for example, a web browser, (2) analyzed at a location separate from where the data was entered, and (3) transmitted back to the location of data entry (or another location) in a readily readable form (e.g., as one or more graphs). Data transfer may be performed using a modem or, as suggested above, the internet.

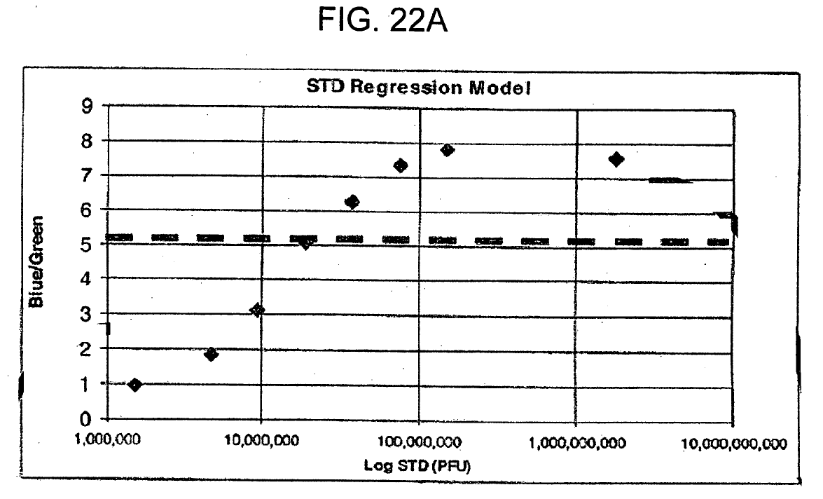

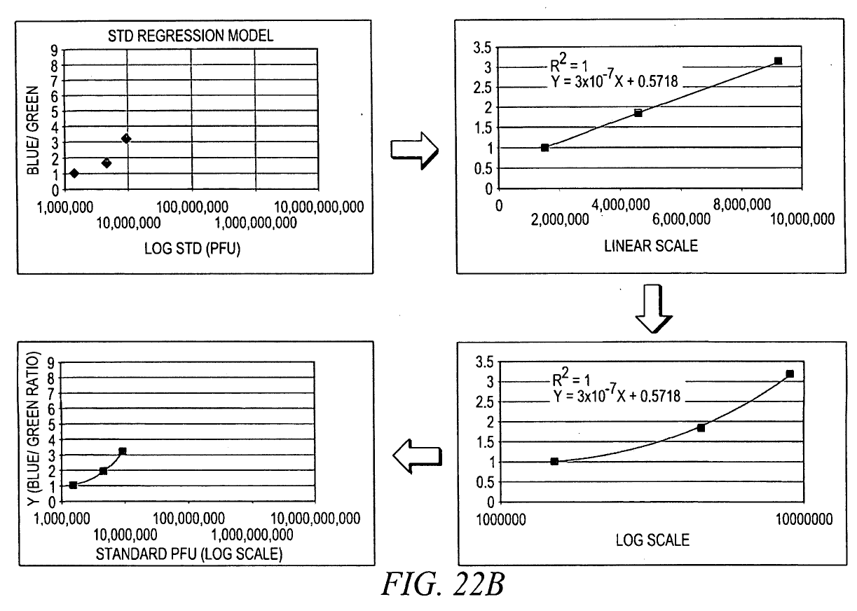

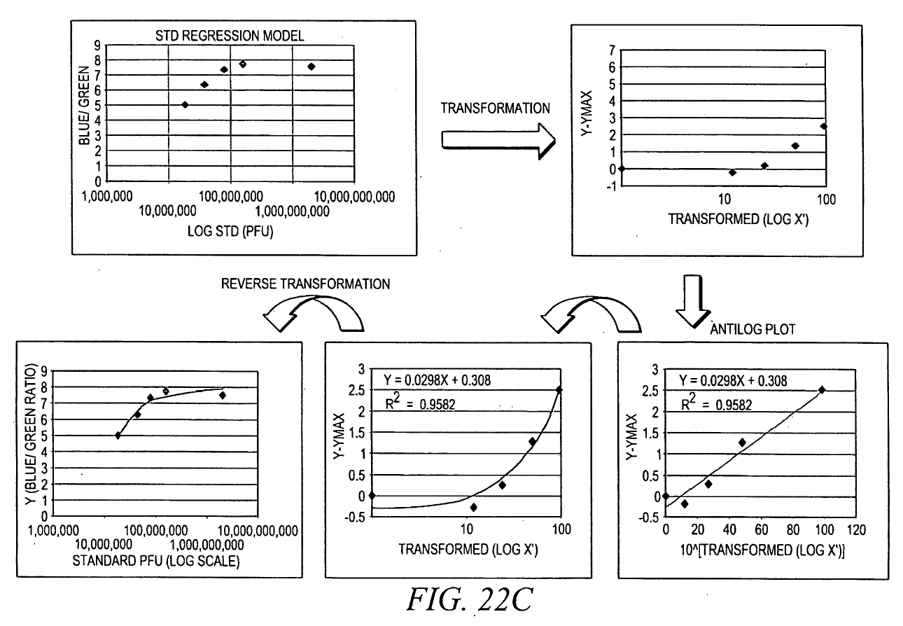

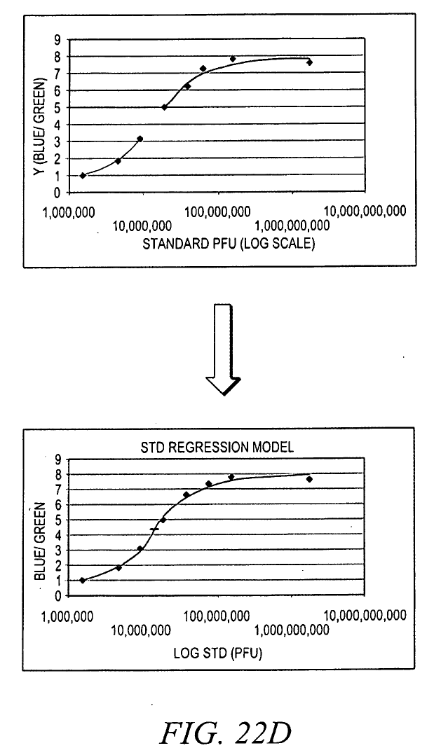

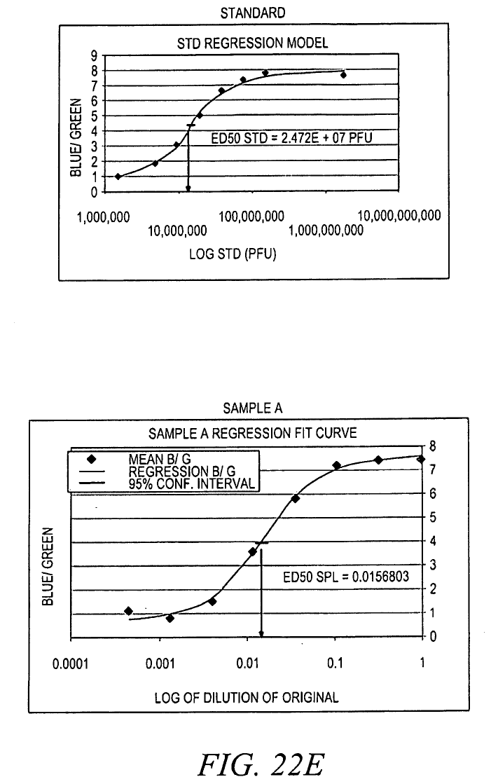

Examples of graphical data generated using methods of the invention are set out in FIG. 22.

### Kits

In another aspect, the invention provides kits that may be used in conjunction with methods the invention. Kits according to this aspect of the invention may comprise one or more containers, which may contain one or more components selected from the group consisting of one or more nucleic acid molecules (e.g., one or more nucleic acid molecules comprising one or more selected nucleic acid sequences operably connected to one or more transcriptional regulatory sequences) and one or more cells comprising such nucleic acid molecules. Kits of the invention may further comprise one or more containers containing cell culture media suitable for culturing cells of the invention, one or more containers containing antibiotics suitable for use in culturing cells of the invention, one or more containers containing buffers, one or more containers containing transfection reagents, and/or one or more containers containing substrates for enzymatic reactions.

Kits of the invention may contain a wide variety of nucleic acid molecules and/or vectors that can be used with the invention. Examples of nucleic acid molecules that can be supplied in kits of the invention include those that contain promoters, signal peptides, enhancers, repressors, selection markers, transcription signals, translation signals, primer hybridization sites (e.g., for sequencing or PCR), recombination sites, restriction sites and polylinkers, sites that suppress the termination of translation in the presence of a suppressor tRNA, suppressor tRNA coding sequences, sequences that encode domains and/or regions (e.g., 6 His tag) for the preparation of fusion proteins, origins of replication, telomeres, centromeres, and the like. Nucleic acid molecules of the invention may comprise any one or more of these features in addition to a transcriptional regulatory sequence as described above.

Nucleic acid molecules to be supplied in kits of the invention can vary greatly. For example a nucleic acid molecule of the invention may comprise a transcriptional regulatory sequence (e.g., a promoter) in juxtaposition with one or more recognition sequences (e.g., recombination sites, topoisomerase sites, restriction enzyme sites, etc.). The recognition sites may then be used to insert a selected nucleic acid sequence into the nucleic acid so as to operably link the transcriptional regulatory sequence with the selected nucleic acid sequence. In some instances, nucleic acid molecules of the invention may further comprise one or more of an origins of replication, one or more selectable markers, and at least one recombination site. For example, nucleic acid molecules supplied in kits of the invention can have a plurality (e.g., two, three, four, five six, seven, eight, nine, ten, fifteen, twenty, etc.) separate recognition sequences that allow for insertion of multiple sequences of interest (that may be the same or different) at multiple different locations of a nucleic acid molecule. Other attributes of vectors supplied in kits of the invention are described elsewhere herein.

Kits of the invention may comprise containers containing one or more recombination proteins. Suitable recombination proteins include, but are not limited to, Cre, Int, IHF, X is, Flp, F is, Hin, Gin, Cin, Tn3 resolvase, φC31, TndX, XerC, and XerD. Preferred recombination proteins and mutant, modified, variant, or derivative recombination sites for use in the invention include those described in U.S. Pat. Nos. 5,888,732, 6,143,557, 6,171,861, 6,270,969, and 6,277,608 and in U.S. application Ser. No. 09/438,358 (filed Nov. 12, 1999), based upon U.S. Provisional Application No. 60/108,324 (filed Nov. 13, 1998). Mutated att sites (e.g., attB 1-10, attP 1-10, attR 1-10 and attL 1-10) are described in U.S. provisional patent application Nos. 60/122,389, filed Mar. 2, 1999, 60/126,049, filed Mar. 23, 1999, 60/136,744, filed May 28, 1999, 60/169,983, filed Dec. 10, 1999, and 60/188,000, filed Mar. 9, 2000, and in U.S. application Ser. No. 09/517,466, filed Mar. 2, 2000, and 09/732,914, filed Dec. 11, 2000 (published as 20020007051-A1) the disclosures of which are specifically incorporated herein by reference in their entirety. Other suitable recombination sites and proteins are those associated with the GATEWAY™ Cloning Technology available from Invitrogen Corp., Carlsbad, Calif., and described in the product literature of the GATEWAY™ Cloning Technology, the entire disclosures of all of which are specifically incorporated herein by reference in their entireties.

Kits of the invention may also comprise one or more topoisomerase proteins and/or one or more nucleic acids comprising one or more topoisomerase recognition sequence. Suitable topoisomerases include Type IA topoisomerases, Type IB topoisomerases and/or Type II topoisomerases. Suitable topoisomerases include, but are not limited to, poxvirus topoisomerases, including vaccinia virus DNA topoisomerase I, E. coli topoisomerase III, E. coli topoisomerase I, topoisomerase III, eukaryotic topoisomerase II, archeal reverse gyrase, yeast topoisomerase III, Drosophila topoisomerase III, human topoisomerase III, Streptococcus pneumoniae topoisomerase III, bacterial gyrase, bacterial DNA topoisomerase IV, eukaryotic DNA topoisomerase II, and T-even phage encoded DNA topoisomerases, and the like. Suitable recognition sequences have been described above. Topoisomerase enzymes are commercially available from, for example, Invitrogen Corporation, Carlsbad, Calif.

In use, a nucleic acid molecule comprising one or more transcriptional regulatory sequence provided in a kit of the invention may be combined with a nucleic acid molecule comprising a selected nucleic acid sequence using recombinational cloning. The nucleic acid molecule comprising one or more transcriptional regulatory sequence may be provided, for example, with two recombination sites that do not recombine with each other. The nucleic acid molecule comprising a selected nucleic acid sequence may also be provided with two recombination sites, each of which is capable of recombining with one of the two sites present on the a nucleic acid molecule comprising one or more transcriptional regulatory sequence. In the presence of the appropriate recombination proteins, the nucleic acid molecule comprising one or more transcriptional regulatory sequences reacts with the nucleic acid molecule comprising the selected nucleic acid sequence in order to form a recombinant nucleic acid molecule containing a transcriptional regulatory sequence operably linked to the selected nucleic acid sequence. When a nucleic acid molecule comprises more than one transcriptional regulatory sequence and multiple pairs of recombination sites, multiple nucleic acid molecules comprising selected nucleic acid sequences, which may be the same or different, may be combined with the nucleic acid molecule comprising multiple transcriptional regulatory sequences to form a nucleic acid molecule comprising multiple transcriptional regulatory sequences operably linked to multiple selected nucleic acid sequences.

Kits of the invention can also be supplied with primers. These primers will generally be designed to anneal to molecules having specific nucleotide sequences. For example, these primers can be designed for use in PCR to amplify a particular nucleic acid molecule. Further, primers supplied with kits of the invention can be sequencing primers designed to hybridize to vector sequences. Thus, such primers will generally be supplied as part of a kit for sequencing nucleic acid molecules that have been inserted into a vector.

One or more buffers (e.g., one, two, three, four, five, eight, ten, fifteen) may be supplied in kits of the invention. These buffers may be supplied at a working concentrations or may be supplied in concentrated form and then diluted to the working concentrations. These buffers will often contain salt, metal ions, co-factors, metal ion chelating agents, etc. for the enhancement of activities or the stabilization of either the buffer itself or molecules in the buffer. Further, these buffers may be supplied in dried or aqueous forms. When buffers are supplied in a dried form, they will generally be dissolved in water prior to use.

Kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

The present invention further relates to instructions for performing one or more methods of the invention (e.g., determining the titer of virus in a solution). Such instructions can instruct a user of conditions suitable for performing methods of the invention. Instructions of the invention can be in a tangible form, for example, written instructions (e.g., typed on paper), or can be in an intangible form, for example, accessible via a computer (e.g., over the internet). Also provided is an instruction set that provides, in part, directions for performing one or more method of the invention. Such an instruction set can instruct a user of conditions suitable for, for example, determining the titer of virus in a solution. Thus, the invention include instructions and instructions sets for performing one or more methods of the invention, as well as methods for performing methods of the invention by following such instructions.

In various aspects, a kit of the invention can contain one or more (e.g., one, two, three, four, five, six, seven, etc.) of the following components: (1) one or more sets of instructions, including, for example, instructions for performing methods of the invention; (2) one or more cells, including, for example, one or more prokaryotic (e.g., bacterial) cells; one or more insect cells; one or more mammalian cells, for example, cells that are adapted for growth in a tissue culture medium, (3) one or more topoisomerases, including, for example, one or more type IA, type IB, or type II topoisomerases, or combinations thereof; (4) one or more nucleic acid molecules, including, for example, one or more vectors, which can be cloning vector or expression vector, one or more transcriptional or translational regulatory elements (e.g., a Shine-Delgarno sequence, a ribosome binding site, a transcriptional promoter and/or enhancer, or a polyadenylation site), any or all of which can be bound to one or more topoisomerases), or one or more coding sequences (e.g., a nucleotide sequence encoding a reporter molecule, detectable transcription or translation product, affinity tag, etc.); (5) one or more cartons, boxes and/or containers for storing and/or transporting kit components (e.g., a box in which to ship components, or a plastic vial in which to store dry, liquid or lyophilized reagents or other kit materials); (6) one or more container containing water (e.g., distilled water) or other aqueous or liquid material; (7) one or more containers containing one or more buffers, which can be buffers in dry, powder form or reconstituted in a liquid such as water, including in a concentrated form such as 2×, 3×, 4×, 5×, etc.); and/or (8) one or more containers containing one or more salts (e.g., sodium chloride, potassium chloride, magnesium chloride, which can be in a dry, powder form or reconstituted in a liquid such as water).

A kit of the invention can include an instruction set, or the instructions can be provided independently of a kit. Such instructions are characterized, in part, in that they provide a user with information related to determining the viral titer of a sample. Instructions can be provided in a kit, for example, written on paper or in a computer readable form provided with the kit, or can be made accessible to a user via the internet, for example, on the world wide web at a URL (uniform resources link; i.e., “address”) specified by the provider of the kit or an agent of the provider. Such instructions direct a user of the kit or other party of particular tasks to be performed or of particular ways for performing a task. In one aspect, the instructions can, for example, instruct a user of the kit as to reaction and/or culture conditions, including, for example, buffers, temperature, and time, to determine the titer of a virus in a sample.

The present invention also provides instructions for performing methods of the invention, such as instructions for a method of preparing a cell comprising a selected nucleic acid sequence operably linked to a transcriptional regulatory sequence, wherein the transcriptional regulatory sequence modulates transcription of the selected nucleic acid sequence when the cell is infected with a virus.

Kits of the invention may also provide instructions for practicing a method of determining the titer of a viral stock, comprising contacting cells with a sample of the viral stock, wherein the cells comprise a selected nucleic acid sequence operably linked to a transcriptional regulatory sequence that is activated by infection of the cell with a virus, and quantifying the amount of the selected nucleic acid sequence that is transcribed.

Kits of the invention may also provide instructions for practicing a method of monitoring progression of a viral infection in a cell, comprising infecting a cell with a virus, wherein the cell comprises a selected nucleic acid sequence operably linked to a transcriptional regulatory sequence, wherein the transcriptional regulatory sequence modulates transcription of the selected nucleic acid sequence when the cell is infected with the virus, and quantifying the amount of the selected nucleic acid sequence that is transcribed.

Kits of the invention may comprise instructions for practicing a method of monitoring a viral infection of a cell population, comprising infecting a cell population with virus, wherein one or more of the cells of the population comprise a selected nucleic acid sequence operably linked to a transcriptional regulatory sequence, wherein the transcriptional regulatory sequence modulates transcription of the selected nucleic acid sequence when the cell is infected with the virus, obtaining a sample of the infected cell population, and quantifying the amount of the selected nucleic acid sequence that is transcribed in the sample.

Kits of the invention may comprise instructions for practicing a method of expressing a polypeptide, comprising providing a cell comprising a selected nucleic acid sequence encoding a polypeptide operably linked to a transcriptional regulatory sequence, wherein the transcriptional regulatory sequence modulates transcription of the selected nucleic acid sequence when a transacting factor is introduced into the cell, and introducing into the cell the transacting factor under conditions causing the expression of the polypeptide.

Such instructions can further include directions for providing conditions such as buffer and salt conditions, as well as temperature and time for performing reactions of the invention as described, for example, elsewhere herein. The instructions of the invention can be in a tangible form, for example, printed or otherwise imprinted on paper, or in an intangible form, for example, present on an internet web page at a defined and accessible URL).

It will be recognized that a full text of instructions for performing a method of the invention or, where the instructions are included with a kit, for using the kit, need not be provided. One example of a situation in which a kit of the invention, for example, would not contain such full length instructions is where the provided directions inform a user of the kits where to obtain instructions for practicing methods for which the kit can be used. Thus, instructions for performing methods of the invention can be obtained from internet web pages, separately sold or distributed manuals or other product literature, etc. The invention thus includes kits that direct a kit user to one or more locations where instructions not directly packaged and/or distributed with the kits can be found. Such instructions can be in any form including, but not limited to, electronic or printed forms.

### Business Methods

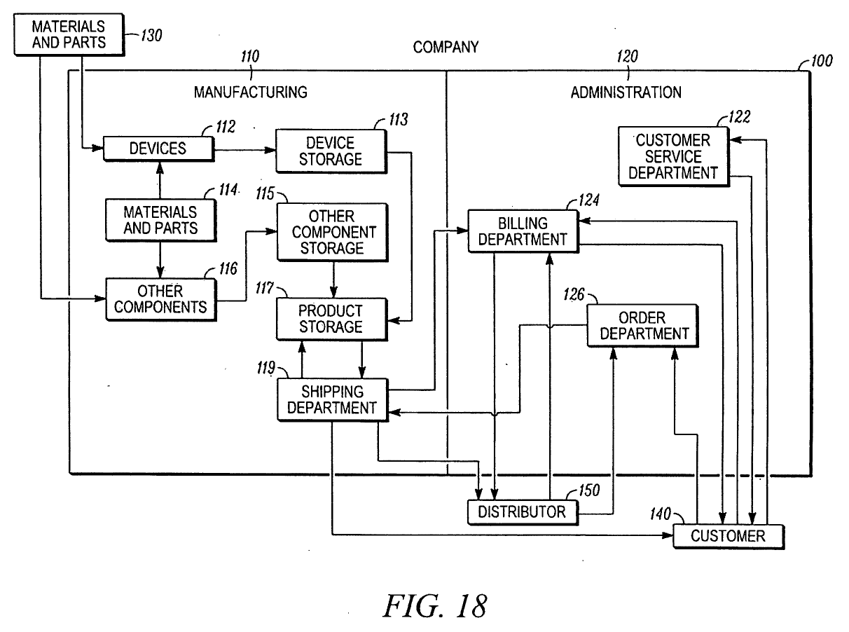

The present invention also provides a system and method of providing company products to a party outside of the company, for example, a system and method for providing a customer or a product distributor a product of the company such as a kit containing materials for determining the titer of a virus in a solution. FIG. 18 provides a schematic diagram of a product management system. In practice, the blocks in FIG. 18 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The product management system as shown in FIG. 18 is exemplified by company 100, which receives input in the form of an order from a party outside of the company, e.g., distributor 150 or customer 140, to order department 126, or in the form of materials and parts 130 from a party outside of the company; and provides output in the form of a product delivered from shipping department 119 to distributor 150 or customer 140. Company 100 system is organized to optimize receipt of orders and delivery of a products to a party outside of the company in a cost efficient manner, particularly instructions or a kit of the present invention, and to obtain payment for such product from the party in a timely manner.

With respect to the methods of the present invention, the term “materials and parts” refers to items that are used to make a device, other component, or product, which generally is a device, other component, or product that company sells to a party outside of the company. As such, materials and parts include, for example, topoisomerases, nucleotides, host cells, polymerases, amino acids, culture media, buffers, paper, ink, reaction vessels, etc. In comparison, the term “devices”, “other components”, and “products” refer to items sold by the company. Devices are exemplified by nucleic acid molecules that are to be sold by the company, for example, vectors, cDNA molecules, open reading frames, regulatory elements, and the like, some or all of which can, but need not, be topoisomerase charged. Other components are exemplified by instructions, including instructions for practicing the methods of the invention (e.g., determining the titer of a virus in a solution). Other components also can be items that may be included in a kit, e.g., a kit product, for example, reagents for manipulating nucleic acid molecules (e.g., performing recombinational cloning and/or topoisomerase mediated joining). Such reagents may include, for example, buffers, salts, cofactors and the like. Other components may include host cells. As such, it will be recognized that an item useful as materials and parts as defined herein further can be considered an other component, which can be sold by the company. The term “products” refers to devices, other components, or combinations thereof, including combinations with additional materials and parts, that are sold or desired to be sold or otherwise provided by a company to one or more parties outside of the company. Products are exemplified herein by kits, which can contain instructions according to the present invention, and one or more nucleic acid molecules, which may be comprise one or more recombination sites, reagents, or combinations thereof.

Referring to FIG. 18, company 100 includes manufacturing 110 and administration 120. Devices 112 and other components 114 are produced in manufacturing 110, and can be stored separately therein such as in device storage 113 and other component storage 115, respectively, or can be further assembled and stored in product storage 117. Materials and parts 130 can be provided to company 100 from an outside source and/or materials and parts 114 can be prepared in company, and used to produce devices 112 and other components 116, which, in turn, can be assembled and sold as a product. Manufacturing 110 also includes shipping department 119, which, upon receiving input as to an order, can obtain products to be shipped from product storage 117 and forward the product to a party outside the company.

For purposes of the present invention, product storage 117 can store instructions for practicing the methods of the invention (e.g., determining the titer of a virus in a solution); or can store kits, which can contain at least one nucleic acid molecule and/or cell line, and, optionally, instructions as disclosed herein; or can store a combination of such instructions and/or kits. Upon receiving input from order department 126, for example, that customer 140 has ordered such a kit and instructions, shipping department 119 can obtain from product storage 117 such kit for shipping, and can further obtain such instructions in a written form to include with the kit, and ship the kit and instructions to customer 140 (and providing input to billing department 124 that the product was shipped; or shipping department 119 can obtain from product storage 117 the kit for shipping, and can further provide the instructions to customer 140 in an electronic form, by accessing a database in company 100 that contains the instructions, and transmitting the instructions to customer 140 via the internet (not shown).

As further exemplified in FIG. 18, administration 120 includes order department 126, which receives input in the form of an order for a product from customer 140 or distributor 150. Order department 126 then provides output in the form of instructions to shipping department 119 to fill the order, i.e., to forward products as requested to customer 140 or distributor 150. Shipping department 119, in addition to filling the order, further provides input to billing department 124 in the form of confirmation of the products that have been shipped. Billing department 124 then can provide output in the form of a bill to customer 140 or distributor 150 as appropriate, and can further receive input that the bill has been paid, or, if no such input is received, can further provide output to customer 140 or distributor 150 that such payment may be delinquent. Additional optional components of company 100 include customer service department 122, which can receive input from customer 140 and can provide output in the form of feedback or information to customer 140. Furthermore, although not shown in FIG. 18, customer service 122 can receive input or provide output to any other component of company. For example, customer service department 122 can receive input from customer 140 indicating that an ordered product was not received, wherein customer service department 122 can provide output to shipping department 119 and/or order department 126 and/or billing department 124 regarding the missing product, thus providing a means to assure customer 140 satisfaction. Customer service department 122 also can receive input from customer 140 in the form of requested technical information, for example, for confirming that instructions of the invention can be applied to the particular need of customer 140, and can provide output to customer 140 in the form of a response to the requested technical information.

As such, the components of company 100 are suitably configured to communicate with each other to facilitate the transfer of materials and parts, devices, other components, products, and information within company 100, and company 100 is further suitably configured to receive input from or provide output to an outside party. For example, a physical path can be utilized to transfer products from product storage 117 to shipping department 119 upon receiving suitable input from order department 126. Order department 126, in comparison, can be linked electronically with other components within company 100, for example, by a communication network such as an intranet, and can be further configured to receive input, for example, from customer 140 by a telephone network, by mail or other carrier service, or via the internet. For electronic input and/or output, a direct electronic link such as a T1 line or a direct wireless connection also can be established, particularly within company 100 and, if desired, with distributor 150 or materials or parts 130 provider, or the like.

Although not illustrated, company 100 may comprise one or more data collection systems, including, for example, a customer data collection system, which can be realized as a personal computer, a computer network, a personal digital assistant (PDA), an audio recording medium, a document in which written entries are made, any suitable device capable of receiving data, or any combination of the foregoing. Data collection systems can be used to gather data associated with a customer 140 or distributor 150, including, for example, a customer's shipping address and billing address, as well as more specific information such as the customer's ordering history and payment history, such data being useful, for example, to determine that a customer has made sufficient purchases to qualify for a discount on one or more future purchases.

Company 1100 can utilize a number of software applications to provide components of company 100 with information or to provide a party outside of company access to one or more components of company 100, for example, access to order department 126 or customer service department 122. Such software applications can comprise a communication network such as the Internet, a local area network, or an intranet. For example, in an internet-based application, customer 140 can access a suitable web site and/or a web server that cooperates with order department 126 such that customer 140 can provide input in the form of an order to order department 126. In response, order department 126 can communicate with customer 140 to confirm that the order has been received, and can further communicate with shipping department 119, providing input that products such as a kit of the invention, which contains, for example, a topoisomerase charged nucleic acid molecule and instructions for use, should be shipped to customer 140. In this manner, the business of company 100 can proceed in an efficient manner.

In a networked arrangement, billing department 124 and shipping department 119, for example, can communicate with one another by way of respective computer systems. As used herein, the term “computer system” refers to general purpose computer systems such as network servers, laptop systems, desktop systems, handheld systems, personal digital assistants, computing kiosks, and the like. Similarly, in accordance with known techniques, distributor 150 can access a web site maintained by company 100 after establishing an online connection to the network, particularly to order department 126, and can provide input in the form of an order. If desired, a hard copy of an order placed with order department 126 can be printed from the web browser application resident at distributor 150.

The various software modules associated with the implementation of the present invention can be suitably loaded into the computer systems resident at company 100 and any party outside of company 100 as desired, or the software code can be stored on a computer-readable medium such as a floppy disk, magnetic tape, or an optical disk. In an online implementation, a server and web site maintained by company 100 can be configured to provide software downloads to remote users such as distributor 150, materials and parts 130, and the like. When implemented in software, the techniques of the present invention are carried out by code segments and instructions associated with the various process tasks described herein.

Accordingly, the present invention further includes methods for providing various aspects of a product (e.g., a kit and/or instructions of the invention), as well as information regarding various aspects of the invention, to parties such as the parties shown as customer 140 and distributor 150 in FIG. 18. Thus, methods for selling devices, products and methods of the invention to such parties are provided, as are methods related to those sales, including customer support, billing, product inventory management within the company, etc. Examples of such methods are shown in FIG. 18, including, for example, wherein materials and parts 130 can be acquired from a source outside of company 100 (e.g., a supplier) and used to prepare devices (e.g., nucleic acid molecules and/or cell lines) used in preparing a composition or practicing a method of the invention, for example, kits, which can be maintained as an inventory in product storage 117. It should be recognized that devices 112 can be sold directly to a customer and/or distributor (not shown), or can be combined with one or more other components 116, and sold to a customer and/or distributor as the combined product. The other components 116 can be obtained from a source outside of company 100 (materials and parts 130) or can be prepared within company 100 (materials and parts 114). As such, the term “product” is used generally herein to refer an item sent to a party outside of the company (a customer, a distributor, etc.) and includes items such as devices 112, which can be sent to a party alone or as a component of a kit or the like.

At the appropriate time, the product is removed from product storage 117, for example, by shipping department 119, and sent to a requesting party such as customer 140 or distributor 150. Typically, such shipping occurs in response to the party placing an order, which is then forwarded the within the organization as exemplified in FIG. 18, and results in the ordered product being sent to the party. Data regarding shipment of the product to the party is transmitted further within the organization, for example, from shipping department 119 to billing department 124, which, in turn, can transmit a bill to the party, either with the product, or at a time after the product has been sent. Further, a bill can be sent in instances where the party has not paid for the product shipped within a certain period of time (e.g., within 30 days, within 45 days, within 60 days, within 90 days, within 120 days, within from 30 days to 120 days, within from 45 days to 120 days, within from 60 days to 120 days, within from 90 days to 120 days, within from 30 days to 90 days, within from 30 days to 60 days, within from 30 days to 45 days, within from 60 days to 90 days, etc.). Typically, billing department 124 also is responsible for processing payment(s) made by the party. It will be recognized that variations from the exemplified method can be utilized; for example, customer service department 122 can receive an order from the party, and transmit the order to shipping department 119 (not shown), thus serving the functions exemplified in FIG. 18 by order department 126 and the customer service department 122.

The methods of the invention also include providing technical service to parties using a product, particularly a kit of the invention. While such a function can be performed by individuals involved in product research and development, inquiries related to technical service generally are handled, routed, and/or directed by an administrative department of the organization (e.g., customer service department 122). Often communications related to technical service (e.g., solving problems related to use of the product or individual components of the product) require a two way exchange of information, as exemplified by arrows indicating pathways of communication between customer 150 and customer service department 122.

As mentioned above, any number of variations of the process exemplified in FIG. 18 are possible and within the scope of the invention. Accordingly, the invention includes methods (e.g., business methods) that involve (1) the production of products (e.g., nucleic acid and/or protein molecules, kits that contain instructions for performing methods of the invention, etc.); (2) receiving orders for these products; (3) sending the products to parties placing such orders; (4) sending bills to parties obliged to pay for products sent to such; and/or (5) receiving payment for products sent to parties. For example, methods are provided that comprise two or more of the following steps: (a) obtaining parts, materials, and/or components from a supplier; (b) preparing one or more first products (e.g., one or more nucleic acid molecules and/or cell lines); (c) storing the one or more first products of step (b); (d) combining the one or more first products of step (b) with one or more other components to form one or more second products (e.g., a kit); (e) storing the one or more first products of step (b) or one or more second products of step (d); (f) obtaining an order a first product of step (b) or a second product of step (d); (g) shipping either the first product of step (b) or the second product of step (d) to the party that placed the order of step (f); (h) tracking data regarding to the amount of money owed by the party to which the product is shipped in step (g); (i) sending a bill to the party to which the product is shipped in step (g); (j) obtaining payment for the product shipped in step (g) (generally, but not necessarily, the payment is made by the party to which the product was shipped in step (g); and (k) exchanging technical information between the organization and a party in possession of a product shipped in step (d) (typically, the party to which the product was shipped in step (g)).

The present invention also provides a system and method for providing information as to availability of a product (e.g., a device product, a kit product, and the like) to parties having potential interest in the availability of the kit product. Such a method of the invention, which encompasses a method of advertising to the general or a specified public, the availability of the product, particularly a product comprising instructions and/or a kit of the present invention, can be performed, for example, by transmitting product description data to an output source, for example, an advertiser; further transmitting to the output source instructions to publish the product information data in media accessible to the potential interested parties; and detecting publication of the data in the media, thereby providing information as to availability of the product to parties having potential interest in the availability of the product.

Accordingly, the present invention provides methods for advertising and/or marketing devices, products, and/or methods of the invention, such methods providing the advantage of inducing and/or increasing the sales of such devices, products, and/or methods. For example, advertising and/or marketing methods of the invention include those in which technical specifications and/or descriptions of devices and/or products; methods of using the devices and/or products; and/or instructions for practicing the methods and/or using the devices and/or products are presented to potential interested parties, particularly potential purchasers of the product such as customers, distributors, and the like. In particular embodiments, the advertising and/or marketing methods involve presenting such information in a tangible form or in an intangible to the potential interested parties. As disclosed herein and well known in the art, the term “intangible form” means a form that cannot be physically handled and includes, for example, electronic media (e.g., e-mail, internet web pages, etc.), broadcasts (e.g., television, radio, etc.), and direct contacts (e.g., telephone calls between individuals, between automated machines and individuals, between machines, etc.); whereas the term “tangible form” means a form that can be physically handled.

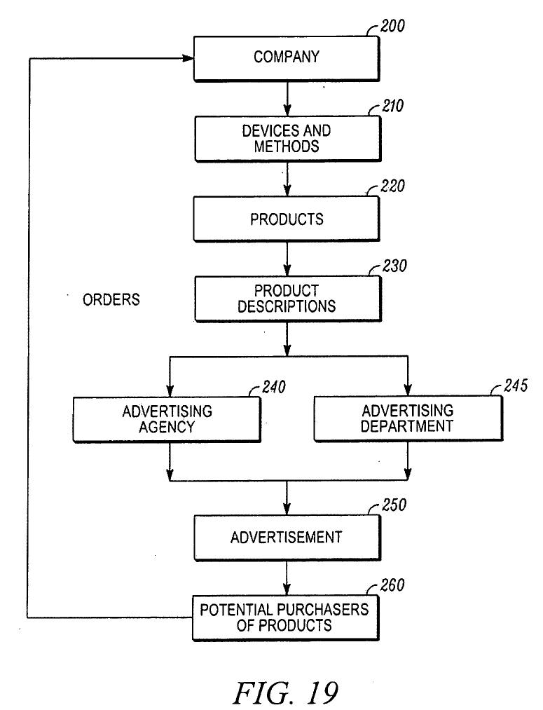

FIG. 19 provides a schematic diagram of an information providing management system as encompassed within the present invention. In practice, the blocks in FIG. 19 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The information providing management system as shown in FIG. 19 is exemplified by company 200, which makes, purchases, or otherwise makes available devices and methods 210 that alone, or in combination, provide products 220, for example, instructions, devices and/or kits of the present invention, that company 200 wishes to sell to interested parties. To this end, product descriptions 230 are made, providing information that would lead potential users to believe that products 220 can be useful to user. In order to effect transfer of product descriptions 230 to the potential users, product descriptions 230 is provided to advertising agency 240, which can be an entity separate from company 200, or to advertising department 260, which can be an entity related to company 200, for example, a subsidiary. Based on the product descriptions 230, advertisement 250 is generated and is provided to media accessible to potential purchasers of products 260, whom may then contact company 200 to purchase products 220.

By way of example, product descriptions 230 can be in a tangible form such as written descriptions, which can be delivered (e.g., mailed, sent by courier, etc) to advertising agency 240 and/or advertising department 250, or can be in an intangible form such as entered into and stored in a database (e.g., on a computer, in an electronic media, etc.) and transmitted to advertising agency 240 and/or advertising department 250 over a telephone line, T1 line, wireless network, or the like. Similarly, advertisement 250 can be a tangible or intangible form such that it conveniently and effectively can be provided to potential parties of interest (e.g., potential purchasers of product 260). For example, advertisement 250 can be provided in printed form as flyers (e.g., at a meeting or other congregation of potential interested parties) or as printed pages (or portions thereof) in magazines known to be read by the potential interested parties (e.g., trade magazines, journals, newspapers, etc.). In addition, or alternatively, advertisement 250 can be provided in the form of directed mailing of computer media containing the advertisement (e.g., CDs, DVDs, floppy discs, etc.) or of e-mail (i.e., mail or e-mail that is sent only to selected parties, for example, parties known to members of an organization that includes or is likely to include potential users of products 220); of web pages (e.g., on a website provided by company 200, or having links to the company 200 website); or of pop-up or pop-under ads on web pages known to be visited by potential purchaser of products 260, and the like. Potential purchasers of products 260, upon being apprised of the availability of the products 220, for example, the kits of the present invention, then can contact company 200 and, if so desired, can order said products 220 for company 200 (see FIG. 18).

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Abstract

The Titer Cell Assay estimates baculovirus titers in 16 hours with very little handling time. The assay uses a Sf21 cell line that was transformed with a plasmid that expresses β-lactamase in response to baculovirus infection. The ratiometric analysis of β-lactamase activity with the substrate CCF2 in combination with a convenient statistical analysis tool, creates a robust assay not affected by Variations in cells, substrate or technique. The assay was shown to be accurate and reproducible between users.

Introduction:

The baculovirus expression system has long been a common tool for expression of recombinant proteins. While baculovirus cloning methods have become as simple as a GATEWAY™ reaction (see, e.g., Invitrogen Corporation, cat. nos. 12537-023, 11828-019, and 10835-031), baculovirus titer technology is still cumbersome and inaccurate. Titers can be obtained by measurement of cell viability/cytopathic effects (CPE), limiting dilution or plaque assays (Reed and Meunch, 1938; Neilson and Smyth, 1992). Each of these methods can require a week or more and significant handling time. Results obtained by different people can vary widely depending on their experience in the subjective assessment of CPE or counting of plaques. Reporter genes have been used to improve the reliability of titer estimation in plaque assay or limiting dilution assays, but these methods still suffer from the labor-intensive process of counting numerous plaques or wells (Cha and Gotoh, 1997; Yahata and Andriole, 2000).

There are two commercial suppliers of kits for estimating baculovirus titer. Both use an antibody against the baculovirus major envelope protein, gp64, in what amounts to an immunologically enhanced plaque assay (Kitts and Green, 1999; Kwon and Dojima, 2002). Both protocols require preparation of virus dilutions, infection of cells, and counting of infected cells or nascent plaques 24 or 48 hours later. A formula is used to estimate the titer based on the experimental relationship between the amount of virus added (or multiplicity of infection, MOI) and the number of plaques that are counted at each dilution. Thus these methods are somewhat faster than a plaque assay but nevertheless require counting numerous plaques.

The Titer Cell Assay described in this example is based on a totally different concept. A cell line was engineered to express the β-lactamase gene from a baculovirus-responsive promoter. Cells infected with more baculovirus express more β-lactamase. The assay estimates the titer of an unknown baculovirus sample relative to a previously titered baculovirus standard using a fluorescence microtiter plate reader. An easy to use spreadsheet tool was created to aid in the analysis of the data. Using this method, baculovirus titers can be obtained in 16 hours in a procedure requiring only 90 minutes of total handling time. This example describes the use of the titer cell assay and shows data that clearly demonstrate superior convenience, accuracy, and reproducibility of the assay compared other titer methods.

Materials and Methods:

Cells and Virus:

All cell lines were maintained in attached or shake cultures in Grace's complete media, supplemented with 10% FBS. Suspension cells were maintained in log phase between 1.0 and 3.5×10⁶ cells per ml. Viruses were amplified from cells grown in suspension. Infections for making virus stocks were performed at a MOI of 0.1. Infections for producing recombinant protein were performed an MOI of 10.

Creation of β-Lactamase Construct:

Based on earlier work, the baculovirus TLP (telokine-like peptide) and Lef-3 (late expression factor-3) promoters were evaluated for use with the β-lactamase gene for titer estimation. Each promoter had previously been cloned as a 1000 base pair (bp) fragment upstream of the initiation codon of each gene and was transactivated by both IE-1 co-expression and baculovirus infection, using a β-galactosidase reporter. pcDNA6V5/HisA (Invitrogen Corporation, cat. no. V22001) was used as a starting plasmid to create Lef-3 and TLP promoter-based β-lactamase constructs. The SV40 promoter/Bsd transcription unit in pcDNA6 (Invitrogen Corporation, cat. no. 12489-027) was replaced with the gp64 promoter/Bsd transcription unit from pIB/V5-His-DEST (Invitrogen Corporation, cat. no. 12550-018) by PCR. The Amp gene from the resulting pcDNA6-gp64/Blast was removed by digestion and religation using AhdI and SspI. The CMV promoter was removed using BglII/NheI, the ends were blunted and the vector religated on itself. This yielded pcDNA6gp64nixAMPnixCMV. The polyA sequence from the baculovirus p10 gene was obtained from pie1Lef5 (Harwood et al., 1998) and inserted into pcDNA6gp64nixAMPnixCMV using XbaI/AgeI, giving the plasmid intermediate pcDNAv.p10polyA. The BlaM gene was added from a BamHI/NotI fragment of pCRBlunt-BlaM. The TLP or Lef3 promoters were then cloned upstream of the BlaM gene by digesting pIBHr5TLP-LacZ with PmeI/HindIII and ligating to produce pTLP (or Lef3)-BlaM. The resulting plasmids had transcription units consisting of the Lef-3 or TLP promoter driving BlaM with an hr5 enhancer and the p10 polyadenylation sequence (FIG. 1).

Transfection and Construction of Cell Lines:

Sf21 cells were grown to log phase and plated at 10⁶ cells per well in a six well plate. Lef-3 or TLP BlaM plasmids (FIG. 1) were transfected into Sf21 cells according to standard protocols (Cellfectin Manual, Invitrogen Corporation, cat. no. 10362-010), and selected for 1 week on 50 μg/μl Blasticidin. Each transfection/selection was repeated once, resulting in four polyclonal cell lines. Each polyclonal line was briefly characterized by performing β-lactamase assays with cells infected with baculovirus. After finding that there were not significant differences between the polyclonal cell lines, clonal cell lines were derived from each by limiting dilution. Cells were diluted to 100, 50, 20, 10, and 5 cells per ml. One hundred microliters of each dilution were plated per well in 96 well plates. After one week, and every few days thereafter, the wells were examined for evidence of cell colonies. Only wells showing a single isolated colony were chosen for expansion. Approximately 30 separate clonal lines were expanded for further analysis.

Preliminary Assay Development:

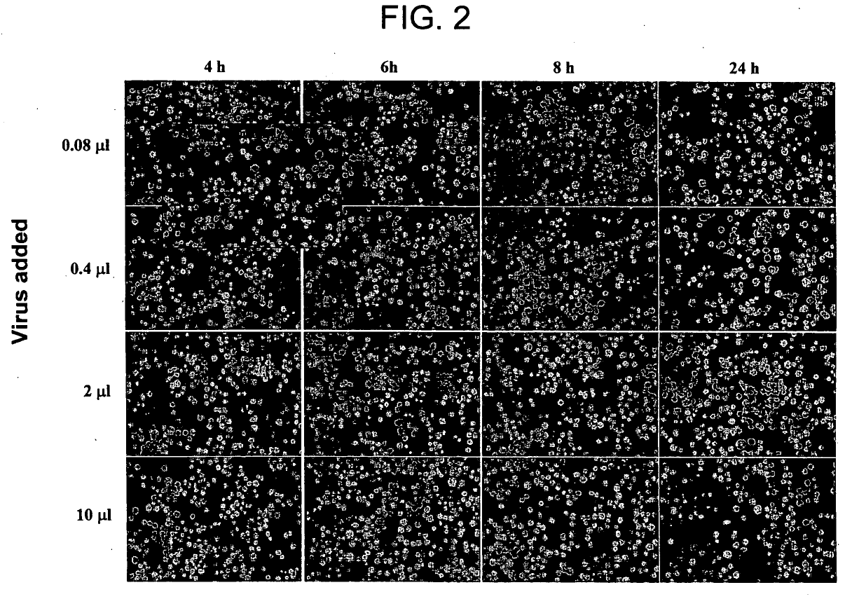

To develop a working protocol for use of β-lactamase with insect cells, the TLP blaM polyclonal cell line was used to optimize assay conditions. Starting with the enhanced loading protocol (GENEBLAZER™ In Vivo Detection Kit, Invitrogen Corporation, cat. no. 12578-134), optimal amounts of substrate and need for probenicid were determined. Preliminary assay conditions were determined by examination of cells by fluorescence microscopy. It was determined that the Enhanced loading protocol suggested in the GENEBLAZER™ In Vivo Detection Kit that uses a 2 μg/ml final concentration of CCF2 and 2 mM probenicid worked well for loading the cells. These conditions were further optimized at a later time (see below). Initial infection parameters were established that demonstrated a correlation between infection time and MOI. Addition of 20 μl of a high titer stock of a recombinant baculovirus to 40,000 Lef3BlaM cells resulted in a clear increase in blue cells within 4 hours after infection. When a 125-fold dilution was added, a qualitative difference in blue cell density was barely discernable after 24 hours (FIG. 2). These conditions were used as a starting point for future quantitative experiments.

TCID₅₀ Method and the Titer Standard

Wild type AcMNPV (Ayers et al., 1994) was amplified from a low MOI infection in Sf 21 cells to a volume of 100 ml, and titered by limiting dilution. Virus high titer stock (HTS), diluted serially in 10-fold series, was applied to 20,000 uninfected Sf21 cells in wells of a 96 well plate. Concentrations were arrayed in columns, giving eight wells per dilution. Dilutions from 1×10² to 1×10¹³ were added in a volume of 10 μl to each well. After one week, 20 μl of supernatant from each well were transferred to 20,000 Sf21 cells in a new plate, and infection scored five days later. This amplified the virus present in each well and ensured that all wells that had any virus at all were scored. Virus titers were estimated using the endpoint dilution method of (Reed and Meunch, 1938). A spreadsheet tool for doing this analysis was outlined by (O'Reilly and Miller, 1992).

Plate Assay:

The CCF2 signal from the β-lactamase assay can be read using a bottom or top read 96 well fluorescence microtiter plate reader. Over several experiments, the optimum cell number for loading and fluorescence reading using a plate reader was found to be 50,000 cells per well in 100 μl media. After allowing 1 hour for cell attachment, 10 μl of various dilutions of virus HTS were added per well. Cells were infected for 16-18 hours unless stated otherwise. Following the infection period, substrate was added to the cells by adding 20 μl of a 6× loading solution (volumes sufficient for one 96 well plate):

### 1880 μl Solution C (24% w/v PEG 400, 18% v/v TR40)

108 μl Solution B (100 mg/ml Pleuronic-F127, 0.1% acetic acid, in DMSO)

### 12 μl CCF2 (1 mM Stock)

120 μl Probenicid (200 mM stock)

After mixing, the 6× loading solution was used immediately. The cells were incubated with substrate at 27° C. for 60-80 minutes after which the plates were read on a Molecular Dynamics Gemini fluorescence plate reader set within the following parameters:

Bottom-read

| | | |
|---|---|---|
| Excitation: | 405 nm ± 10 | nm |
| Emission Filter (Blue) | 460 ± 20 | nm |
| Emission Filter (Green) | 530 nm ± 15 | nm |

A top-read protocol was also developed, although the bottom read method is preferable (see below). To use the assay using a top-read, the media and substrate were removed after substrate incubation, and the cells were washed once with 200 μl Grace's Insect Tissue Culture media, unsupplemented (Invitrogen Corporation, cat. no. 11595-030). One hundred microliters of Grace's incomplete was added to the washed cells and then the plate was read from the top, using the same wavelength parameters.

A detailed protocol appears below in Appendix 1.

Analysis Method:

The data output from the plate assay was analyzed using a custom-designed, Excel-based macro (Appendices 1 and 3). The spreadsheet estimates the virus concentration of the HTS that gives a 50% response in the B/G (blue/green) ratio (ED₅₀). The ED₅₀ values for the standard and unknown virus HTS are compared. Since the titer of the standard HTS is known, the dilution of unknown virus HTS that gives the ED₅₀ provides an estimate of its titer. The spreadsheet uses a biphasic least squares analysis to estimate a model for the curve and provides a 95% confidence interval for the titer estimate (Appendices 1 and 3).

Results:

Selection of Clones:

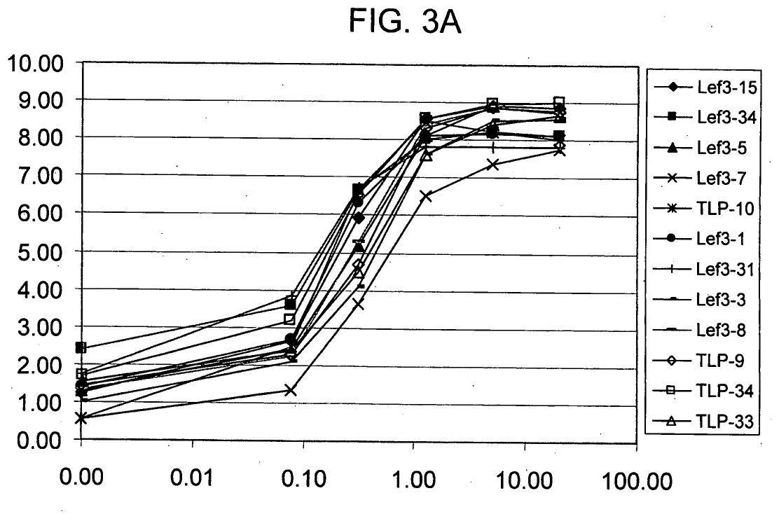

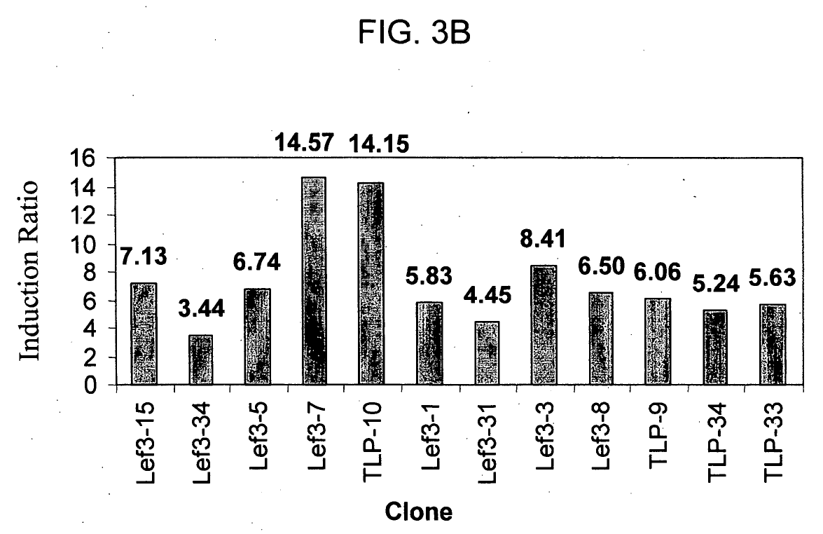

Clonal cell lines were analyzed using β-lactamase assays read on a plate reader. Typically, the virus stock was diluted in 4× or 5× serial increments and applied to cells in a volume of 10 or 20 μl. Clonal cells amplified to T-75 scale were sloughed using a stream of media. Equivalent numbers of cells were plated in 24 wells (per clone) of 96 well plates. Each clone was tested with six virus HTS concentrations in quadruplicate. FIG. 3A shows an aggregate of 12 clones tested in this manner. The response of B/G ratio as a function of virus input was very consistent between clones and showed a typical sigmoid shape when the X-axis (virus added) was log-transformed. Approximately 30 clonal cell lines were compared based on the induction ratio, calculated as the B/G ratio obtained at saturating amounts of virus divided by the B/G ratio obtained with no virus added (FIG. 3B). Two clones clearly had higher induction ratios, TLP-10 and Lef3-7. Ultimately, TLP-10 cells were chosen for use in the titer kit because the induction ratio was, over several experiments, higher than the Lef3-7 cells.

Analysis Method:

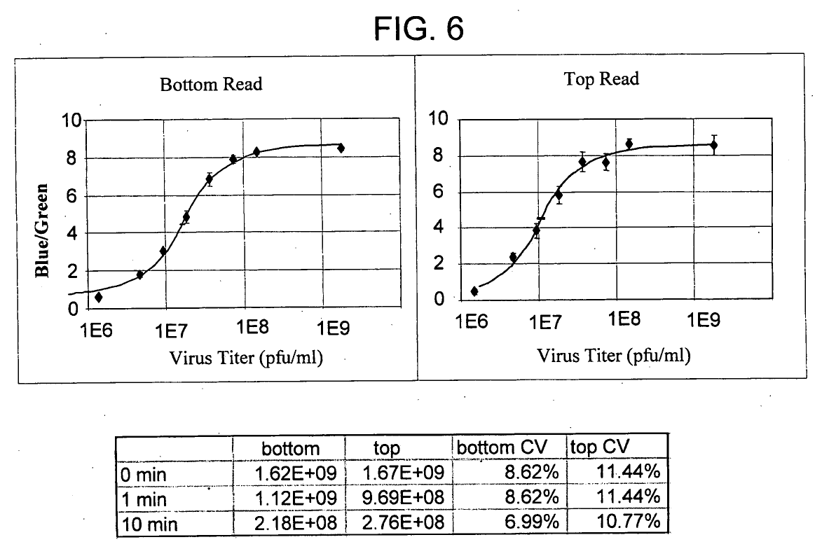

The substrate CCF2 is cleaved by β-lactamase. The reaction is monitored by fluorescence-resonance energy transfer (FRET) as a ratio between the fluorescence of the cleaved substrate (blue, emission at 460 nm) over the emission of the non-cleaved substrate (green, emission at 530 nm). The analysis used in this assay measures the blue/green ratio (B/G) output of β-lactamase in relation to the amount of virus HTS added to the cells. When no virus is added, the B/G is typically 0.5-0.8. When saturating amounts of virus are added, the B/G is typically 8-10. A plot of log [virus concentration] vs. B/G ratio yields a sigmoid shaped curve (see for example, FIG. 6, bottom read). A mathematical model was developed that estimates the titer of unknowns relative to a standard virus by linear regression, using transformed data from a sigmoid curve (Appendices 1 and 3). Data are input as two 8×12 grids of data corresponding to the emission output from blue and green channels from the plate reader. The model calculates the ratio of the blue channel, divided by the green channel and outputs these in a separate 8×12 grid. It presents a scatter plot for the data for the standard virus and two unknowns, allowing the operator to scan for outliers. Outliers can be eliminated by deleting their values from the B/G ratio grid. On the output page, graphs corresponding to average B/G ratio vs. virus dilution are plotted and the estimates for the titer of the unknowns are displayed with upper and lower 95% confidence limits. The confidence limits are determined from a pooled variance calculated by the model. The variance is reflected in the coefficient of variation, which can be viewed in a separate tab. The model used for assay development was originally written as an Excel workbook (Excel, version 2000). The model will be adapted for use on a website with instructions for use. The outputs of a web site accessible version may be essentially be the same as the Excel workbook. Further, the Excel workbook may be stored with this document in a directory file. The analysis method and the algorithm are described in Appendices 1 and 3, respectively.

Cell Number:

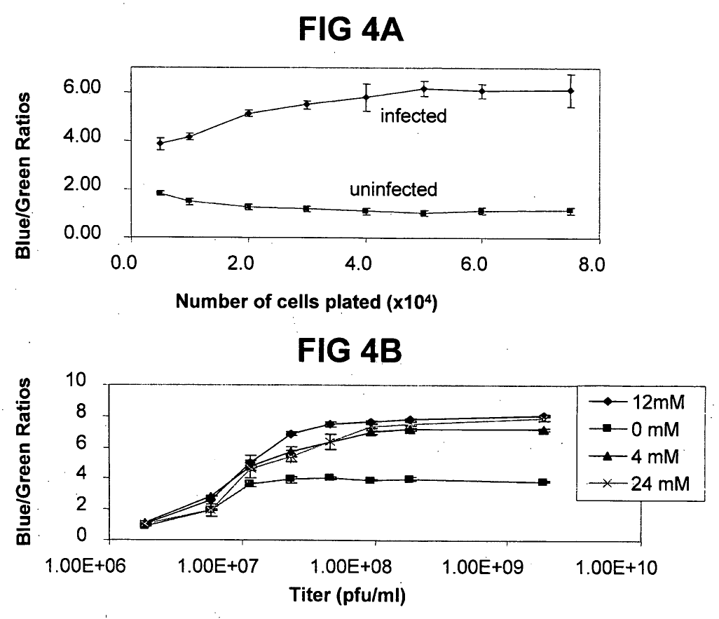

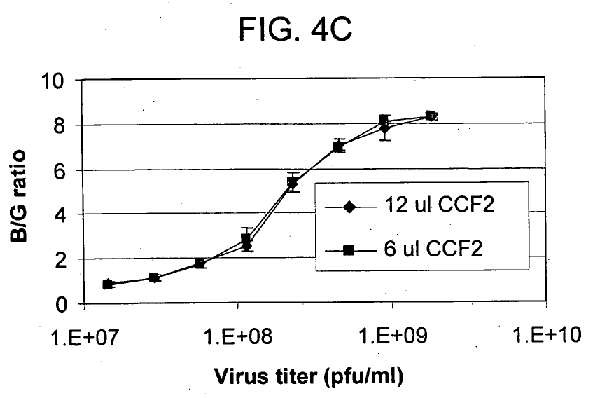

The number of plated cells per well was optimized. Varying numbers of TLP-10 cells were plated in wells of a 96 well plate. Ten microliters of AcMNPV standard virus HTS were added to the wells, and B/G ratios were examined the following morning. The B/G ratio of infected cells increased steadily with cell number, reaching a plateau at 50,000 cells per well (FIG. 4A). Fifty thousand cells per well were used for subsequent experiments.

Probenicid and CCF2:

The enhanced loading protocol is an alternative protocol in the GENEBLAZER™ In Vivo Detection Kit for mammalian cells that do not load efficiently. It differs from the standard protocol by using 2 μg/ml instead of 1 μg/ml CCF2, and uses probenicid to prevent transport of substrate out of the cells. The requirement for higher substrate concentration and the optimal amount of probenicid were tested. Cells were plated at 50,000 cells per well and varying concentrations of virus were used for infection. After 16 hours, the cells were loaded with 20 μl of 6× loading solutions with 0, 4, 12, and 24 mM probenicid (FIG. 4B). As suggested by preliminary microscopic evaluation, probenicid was required. With no probenicid, the B/G ratio peaked at about 4, half of that obtained with the optimum concentration, 12 mM. Twelve millimolar (2 mM final concentration) is the concentration used for the enhanced protocol, and therefore was used for all subsequent experiments and the kit design. The CCF2 concentrations in the standard and enhanced protocols were also tested (FIG. 4C). Standard curves generated using 1 or 2 μg/ml CCF2 were indistinguishable. The lower concentration will be used for the kit.

Time of Infection:

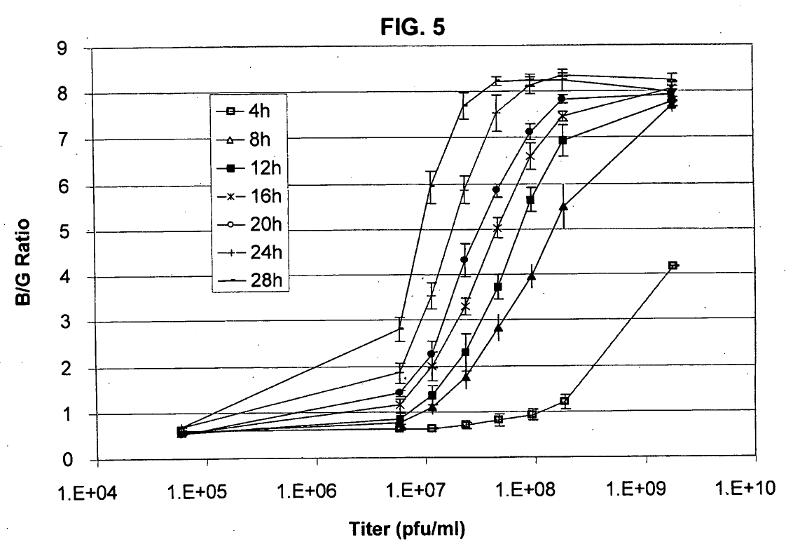

The optimum infection time for the titer assay was determined by infecting cells with serial dilutions of wtAcMNPV and loading and reading the plates at 4 hour intervals following infection. A detectable CCF2 response was obtained after 4 hours, but the B/G ratio was less than half of maximal at the highest virus concentration (FIG. 5). After 8 hours, the B/G ratio was nearly maximal at the highest concentration. Saturation was observed at 12 hours, but a 12-hour infection period is not convenient for customer assays. At 16 hours, saturation was observed at the highest concentration and a clear linear response (log [virus]) was seen at intermediate concentrations. At 20 through 28-hour infection periods, the curve increasingly steepened and shifted to the left and saturation was observed at lower concentrations of virus, possibly suggesting secondary infection by budded virus produced during the infection.

Top Read Protocol:

A top read protocol was considered important because not all potential users will have a fluorescence, plate reader with bottom-read capability. The protocol for top reading is identical to that for bottom reading except that the media and substrate must be removed after loading but before measurement. This was accomplished with a single washing of the cells with 200 μl of Grace's incomplete media. PBS will also work for this purpose. Following the wash, the media was replaced with 100 μl of Grace's incomplete media. The plate was read using the same parameters used for bottom reading. To test, the titers of three different HTS were estimated against the AcMNPV standard HTS using both bottom and top read protocols (FIG. 6). The titer estimates between the two protocols were very similar, however the coefficient of variation (a measure of variability in the data) was higher using the top read protocol (FIG. 6). It was concluded that the top read protocol is useable but the bottom read protocol is preferable. The bottom read protocol is faster and more reliable than the top read protocol.

Effect of Cell Growth Phase:

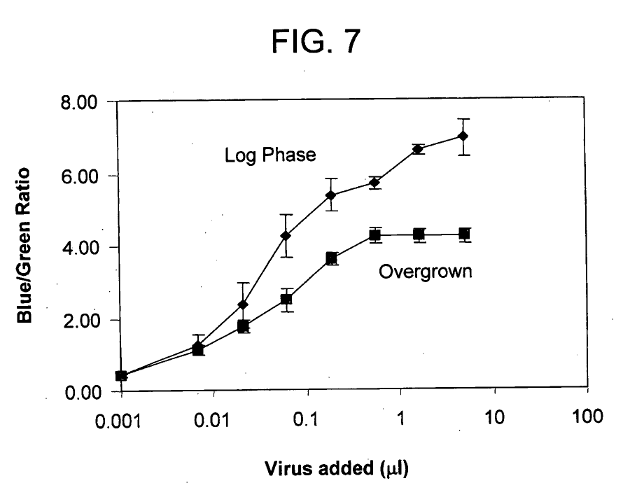

The assay was performed in a variety of sub-optimal ways in an attempt to predict some of the problems users (e.g., customers) might encounter. While minor variations in cell number and substrate are internally corrected by ratiometric detection (data not shown), effective infection and virus gene transcription assume that host cells are healthy and actively growing (i.e., in log phase). The growth of the cells was examined at very low cell densities. The cells doubled in 24 hours to 5×10⁵ cells per ml, the minimum concentration required to plate 50,000 cells per ml in 100 μl of media. Thus it was assumed that there would not be a problem in using cells that were “under grown”. Cells that had “overgrown” were also tested. Paired 50 ml cultures were monitored for cell number and viability. One flask was split at appropriate intervals to keep the cells in log phase between 1.0 and 3.5 million cells per ml. The other flask was growing in log phase at 2×10⁶ cells per ml at 95% viability on a Friday but was not split. On Monday, the cells were at 3.4×10⁶ cells per ml at 90% viability and at 3.1×10⁶ at 78% viability on Tuesday. These cells were then used to create titer curves and compared with the cells that were maintained in log phase. FIG. 7 shows that the cells that had been allowed to overgrow had different infection kinetics and B/G ratio plots than the log phase cells. The overgrown cells saturated at lower virus concentrations and gave lower overall B/G ratios than did the log phase cells. Thus, cells that are growing in log phase should be used.

Effect of Freeze/Thaw Cycles on Virus Titer:

Baculovirus is relatively stable when stored in the dark under a variety of temperature conditions (Jarvis and Garcia, 1994). The effect of freeze/thawing on baculovirus titer was tested, reasoning that the titer of the baculovirus standard would be more stable frozen than at 4° C. 200 μl was aliquotted into three separate tubes. One tube was kept at room temperature (RT) (30 minutes total before use), one was freeze/thawed once, and the last tube was freeze/thawed three times. Freezing was accomplished by placing the tubes on dry ice for 5 minutes; tubes were thawed at room temperature. Tubes were left at RT after thawing until use (maximum of 30 minutes). Titer was unaffected by freezing once or three times. Thus, virus stocks will be stored at −20° C. in aliquots and shipped the kit.

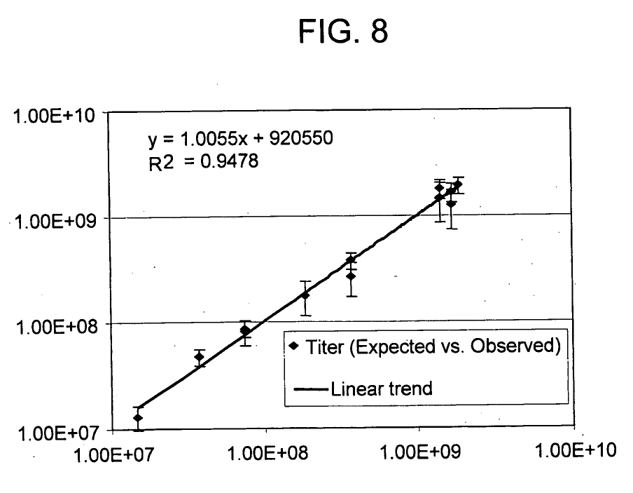

Predictive value:To test the predictive value of the Titer Cell Assay, AcMNPV, GSTManI, and Melittin ManI recombinant baculovirus HTS were titered by limiting dilution. Using the wt AcMNPV HTS as a standard in the Titer Cell Assay, the titers of the two recombinant HTS and dilutions of the wtAcMNPV HTS were then estimated using the Titer Cell Assay. The wtAcMNPV virus was diluted to 0.9, 0.75, 0.4, 0.2, and 0.1 in media and each concentration as well as each recombinant HTS were treated as unknowns in the assay. This allowed for the use a virus of known titer (relative to the undiluted stock) that we could estimate with the titer assay. The expected titers (based on limiting dilution or the predicted titer based on the dilution factor) were plotted against the observed titer, for both the dilutions of wtAcMNPV and the two recombinant HTS (FIG. 8). Overall, there was an excellent correlation between expected titer (based on the dilution factor or limiting dilution assay) and the titer calculated from the Titer Cell Assay, with a slope of 1.05 and a R² of 0.93, demonstrating a highly significant, one for one correspondence between the observed and expected titers, for the AcMNPV dilutions and the virus recombinants.

Assay Test:

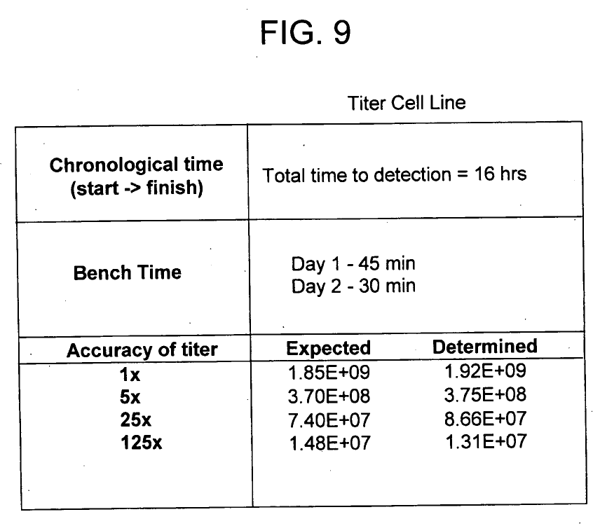

The four virus samples were tested with the Titer cell assay. The results are tabulated in FIG. 9. Chronological time, bench time, qualitative ease of use, and the accuracy of the titers obtained was assessed. Titer estimation using the Titer Cell Assay required only 16 hours with approximately 1.25 hours of handling time.

The titers of the four virus concentration samples described above were analyzed. The Titer Cell Assay closely estimated the virus concentration of all three dilutions (the undiluted virus was also used as the standard, and thus was not an independent sample).

Alpha Test:

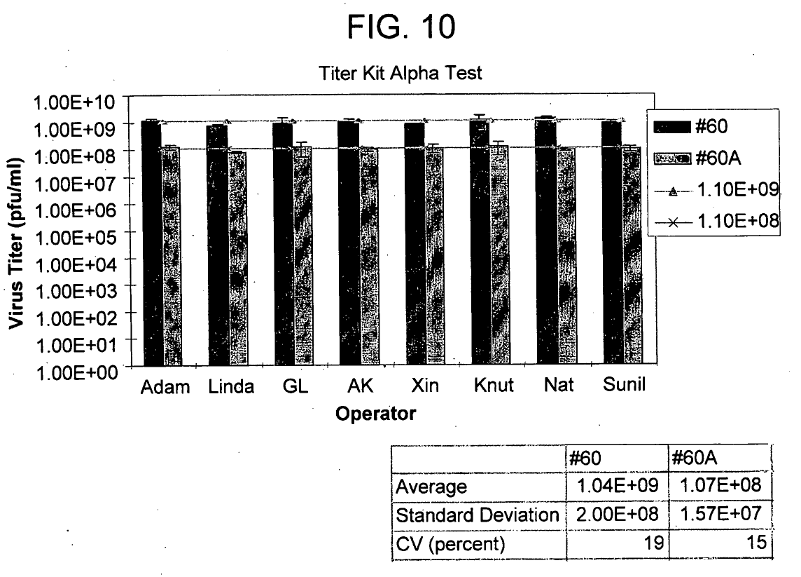

A key shortcoming with many titer methodologies is that results' obtained from the same virus sample between different users can vary widely. Limiting dilution methods ultimately require scoring if cells in a given well “look infected”. This is often subjective and dependent on how skilled the operator is in identifying cytopathic effects of virus infection, among other factors. Likewise, plaque assays involve exhaustively counting all the plaques on a plate at multiple dilutions. Results can be subjective and vary significantly depending on the attentiveness of the operator, the quality of the cell monolayer and agarose overlay, etc. The Titer Cell Assay is not dependent on subjective measures. The cells are plated and infected according to a well-defined set protocol, and the readout is performed by machine. To examine the consistency of the Titer Cell Assay between users, eight lab personnel were chosen to participate in an alpha test of the Titer Cell Assay. Each participant was provided with a protocol, three virus samples (a control and two unknowns) and a plate of TLP-10 cells that had been seeded earlier in the day. The cells had been maintained in log phase and were between 1.5 to 3×10⁶ cells/ml with 95% viability at the time of plating. Each participant prepared virus dilutions and prepared and added the loading solution. Sample A was a 60% dilution of the wtAcMNPV HTS while Sample B was a tenfold dilution of Sample A. All eight participants successfully completed the protocol. Of 16 total titer estimates, 13 fell estimated the expected titer within the 95% confidence interval. The pooled coefficient of variation between participants was 19% for the 60% sample and 15% for the 6% sample (FIG. 10).

Estimate of UV Treated Virus Samples:

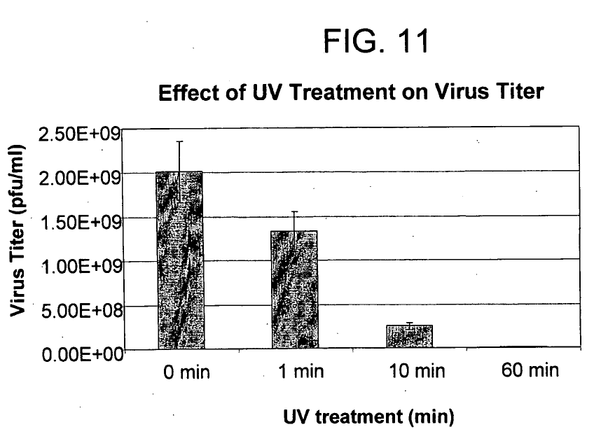

One advantage of the Titer Cell Assay over titer methods that detect DNA in a virus supernatant (i.e., qPCR) is that only infectious virus can be detected. wt AcMNPV HTS was treated with short wave UV light (254 nm) for various amounts of time and then measured the remaining titer. As can be seen, treatment of the virus for as little as one minute reduced the titer significantly. Treatment for 10 minutes caused a 40-fold decrease in titer, and treatment for 60 minutes lowered the titer below that which could be accurately detected by the assay (FIG. 11). Recombinant protein expression using the Titer Cell Line:The ability of the titer cell line to express recombinant protein via baculovirus infection was evaluated. Users could conceivably use the B/G readout from these cells to confirm if their infection was effective. Two samples of virus were used, one that was treated with short-wave UV for 10 minutes, as described above, and one that was untreated. The titer of each sample was estimated using the Titer Cell Assay. Wells of three six well plates were seeded with 2×10⁶ TLP cells and infected with either the MeI SfManI 1-24 baculovirus or the UV treated virus at an MOI of 10 (assuming the titer prior to UV treatment). This simulated what a user might experience if they used virus of unexpectedly low titer. Plates 1 and 2 were loaded with CCF2 at 24 and 48 hours after infection, respectively, and examined by fluorescent microscopy. At 72 hours post-infection, infected cells in the third plate were lysed in SDS PAGE buffer and examined by SDS-PAGE/Western blot using anti-V5 sera. From the titer analysis, a 10 minute treatment of the virus with short wave UV light caused the titer to drop from 2.2×10⁹ to 5.4×10⁷, or approximately 40 fold.

Cells infected with untreated virus were nearly all blue 24 hours and 48 hours post-infection. A clear difference was observed in cells infected with UV treated virus. At 24 and 48 hours post-infection many more uninfected cells were observed. The amount of protein produced by these cells was much less than produced in cells infected with untreated virus. Thus, the β-lactamase assay to check the progress of infection.

2. Discussion

Optimal use of the baculovirus expression system requires infection of insect cells with a defined multiplicity of infection (MOI). Whether cells are infected for virus production at a low MOI, or infected for protein production at a high MOI, optimal results require infection with the correct amount of virus. Thus titer assays ideally should be both accurate and consistent. Current methods for estimating baculovirus titers are time consuming. Both plaque assays and limiting dilution assays require scoring of infected cells based on subjective criteria. While use of reporter genes makes identification of infected cells easier (Yahata and Andriole, 2000; Cha and Gotoh, 1997), enumeration of plaques or infected wells is still laborious and time consuming. In addition, use of such viruses requires the expression of an additional gene product that may interfere with downstream processes. While immunological detection of virus gene products does not require expression of an exogenous gene, such assays do require multiple wash steps, overlays, and counting of infected cells or cell foci (Kwon and Dojima, 2002; Kitts and Green, 1999). The Titer Cell Assay described here circumvents these problems. The assay requires only three addition steps (plate cells, add virus, add substrate), no wash steps, and the output is read on a fluorescence plate reader. An analysis tool provides the titer estimate with a statistically meaningful error estimate.

The Titer Cell Assay estimates the titer of an unknown virus relative to standard baculovirus of known titer. One reason that wild type AcMNPV was chosen as the control virus is because it can be titered by limiting dilution with greater accuracy (polyhedrin positive phenotype) than polyhedrin-negative recombinants. No differences are expected between wt and recombinant baculoviruses in this assay because the entire assay is performed during the early phase of infection, prior to expression of recombinant protein from the very-late polyhedrin promoter. This contention was supported by the excellent agreement between the limiting dilution assays and the Titer Cell Assay for two virus recombinants (FIG. 8). Some commercial baculovirus vectors (i.e., pAcP(+)IE1-1 from Novagen) are designed to'express the gene of interest during the early phase of infection. For these vectors, the nature of the recombinant gene may interfere with the assay. The Titer Assay may not be compatible with other baculoviruses (i.e., LdMNPV, OpMNPV, or BmMNPV) because transactivation of the AcTLP promoter may be specific for the AcMNPV IE-1 protein.

The cells were loaded with substrate and read at 16 hours post-infection prior to the production of budded virus from infected titer cells, ensuring that secondarily produced virus did not re-infect cells and potentially skew the results. By 28 hours post infection, the β-lactamase response curve shifted significantly to the left and saturated at much lower virus concentrations, suggesting that secondary infection occurred at later times post-infection (FIG. 5). One advantage of the Titer Cell Assay is that it measures only infectious virus particles. Virus damaged by UV treatment gave lower titers than untreated virus. While not tested, virus titered by direct detection of virus nucleic acid (e.g, qPCR) would not be expected to distinguish between infectious and non-infectious virus. However, the Titer Cell Assay may not discriminate against defective interfering particles (DIPS). DIPS are virions that have major deletions of portions of their genomes that are not required for DNA replication. What effect DIPS might have on the Titer Cell Assay was not studied, although if virus entry and early transcription functions are still intact, such DIPS may read in the Assay. Maintenance of low passage virus stocks is the best means available to guard against the accumulation of DIPS.

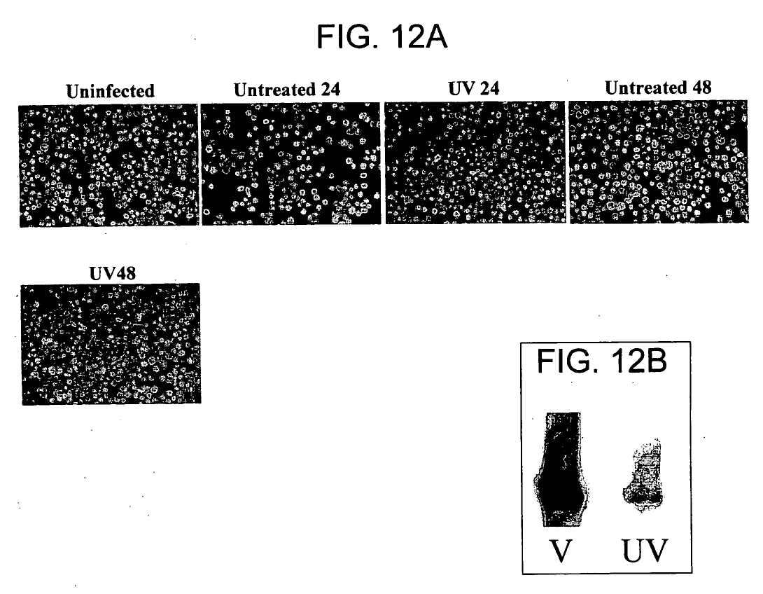

The titer cell line can also be used as a self-reporting expression cell line. Recombinant protein was readily expressed using the titer cell line (FIG. 12), and the progress of infection was easily monitored by removing a sample of infected cells and loading them with CCF2. This allows a simple means of determining if cells are infected without having to wait to determine expression of recombinant protein.

### REFERENCES

Ayers et al, (1994). “The Complete DNA Sequence of Autographa californica Nuclear Polyhedrosis Virus.” Virology 202:586
Cha, H. J., T. Gotoh, et al., (1997). “Simplification of titer determination for recombinant baculovirus by green fluorescent protein marker.” BioTechniques 23(5): 782-786.
Harwood, S. H., L. L1, et al., (1998). “AcMNPV late expression factor-5 interacts with itself and contains a zinc ribbon domain that is required for maximal late transcription activity and is homologous to elongation factor TFIIS.” Virology 250: 113-134.
Jarvis, D. L. and A. Garcia (1994). “Long-term stability of baculoviruses stored under various conditions.” Biotechniques 16(3): 508-513.
Kitts, P. A. and G. Green (1999). “An immunological assay for determination of baculovirus titers in 48 hours.” Analytical Biochemistry 268: 173-178.
Kitts, P. A. and R. D. Possee (1993). “A method for producing recombinant baculovirus expression vectors at high frequency.” BioTechniques 14(5): 810-817.
Kwon, M. S., T. Dojima, et al., (2002). “Development of an antibody-based assay for determination of baculovirus titers in 10 hours.” Biotechnol. Prog. 18: 647-651.
Neilson, L. K., G. K. Smyth, et al., (1992). “Accuracy of the endpoint assay for virus titration.” Cytotechnology 8: 231-236.
O'Reilly, D. R., L. K. Miller, et al., (1992). Baculovirus Expression Vectors a Laboratory Manual. New York, W.H. Freeman Co.
Reed, L. J. and H. Meunch (1938). “A simple method of estimating fifty percent endpoints.” Am. J. Hygiene 27(3): 493-497.
Yahata, T., S. Andriole, et al., (2000). “Estimation of baculovirus titer by β-galactosidase activity assay of virus preparations.” Biotechniques 29(2): 214-215.

### APPENDICES

Appendix 1. Exemplary Titer Cell Assay Protocol

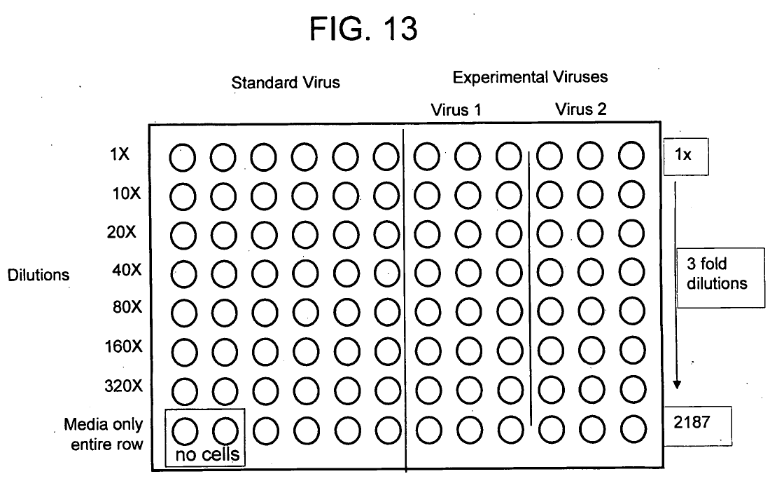

Materials needed:
Microtiter plate reader equipped with correct filters (455 and 530 nm)
Standard Virus 200 μl per two assays
Unknown Virus (200 μl)
1.7 ml Eppendorf tubes and rack
repeating pipettor (preferable)
multichannel pipettor (preferable)
multichannel pipettor reservoirs
Black sided, clear bottom 96 well plates (see FIG. 13)
Log phase titer cells
Grace's complete insect media (Invitrogen Corp., cat. no. 11595-0²²) with 10% Fetal Bovine Serum (Invitrogen cat. No. 16140-014)

Cell Plating:

1. Use cells that are in log phase. (˜1.5-2.5*10⁶ cells/ml).

2. Seed each well in a 96 well black plates (see FIG. 13), with clear bottom wells with 50,000 cells/well in a 100 μl volume. DO NOT PUT CELLS IN WELLS H1 and H2 (lower left corner). Put media only in these wells. Let cells attach 1-4 hours.

Standard Curve and Virus Dilutions:

While cells are attaching, prepare dilutions of the standard and unknown viruses. You will prepare a 10× dilution of standard virus and then prepare a 2× dilution series starting with the 10× dilution (see diagram). For the experimental viruses, you will prepare a 3× dilution series (eight tubes total), starting with the undiluted virus supernatant. Take care to pipette accurately. You may store the dilutions at 4 degrees in the dark if necessary.

1. Dilute concentration standard: Remove 25 ml Grace's complete media to a 50 ml conical tube. Place eight 1.5 ml epi-tubes in a rack. Aliquot 200 μl of the standard virus in the first tube. Place 180 μl media (from the 50 ml conical) in the second tube. Place 100 μl of media in the remaining six tubes. Transfer 20 μl of virus from the first tube (straight virus stock) to the 180 μl in the second tube. Vortex. This gives a 10× dilution of the virus standard. Transfer 100 μl of the 10× virus to the third tube, vortex (20×). Transfer 100 μl of the 20× and transfer to the next tube, vortex (40×). Repeat this procedure for all but the last tube. Leave only media in that tube.

2. Dilute test virus: Place eight epi-tubes in your rack. Place 240 μl of test virus in the first tube. Place 160 μl of media in the remaining tubes. Transfer 80 μl of media from the first tube to the second tube, vortex. Transfer 80 μl from the second tube to the third tube, vortex. Repeat for the remaining tubes.

Infection (at the end of the day):

Before going home for the day, add 10 μl of each virus dilution to the wells as shown. Use a repeating pipettor if available. Alternatively, you can use a manual pipettor. Take care to place each pipette tip into the media of each well so that all of the liquid is dispensed into the media in the well, otherwise, virus will not be delivered to the wells evenly. Rotate the plate by hand periodically as you add the virus solutions to distribute the virus solution evenly in the wells.

Swirl the plate by hand one last time and then place in a 27° C. incubator overnight (no more than 18 hours).

The following morning, perform the β-lactamase assay. You will make up a 6× working stock, add to the wells, and then read the wells 1 hour later.

1. Make up 2 ml 6× β-lactamase assay solution per plate (must used immediately):

### 108 μl Solution B

### 12 μl CCF2

mix, then add
120 μl probenicid

### 880 μl Solution C

2. Add 20 μl to each well using multichannel pipettor. Volume is sufficient to use a reservoir. Avoid creating bubbles.

3. Incubate for 60 minutes at room temperature in the dark.

Detection on bottom read plate reader, Molecular Dynamics Gemini Spectra Max.

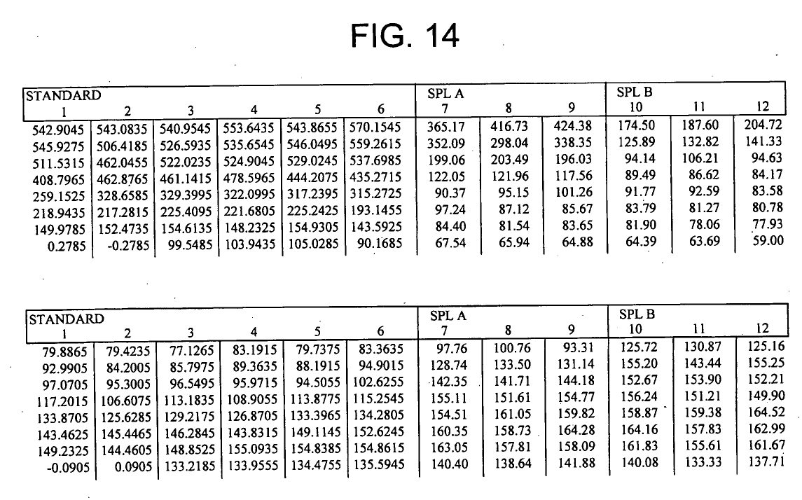

Set up the plate reader for dual wavelength, (405 excitation, 455/530 emission), bottom read, according to the manufacturer's instructions. Set up the template, designating wells H1 and H2 as blanks. The remainder of the plate can be designated as unknowns. Once the template is completed, place the plate without the lid into the machine. Click the READ box. Following the read, go to FILE, SAVE AS, give a file name in your directory, and save. Then go to FILE, EXPORT. This will save the file in text format. Open the spreadsheet tool and go to the input tab (lower left, below the body of the spreadsheet). Paste the blue and green channel data grids into the blue and green channel fields (FIG. 14).

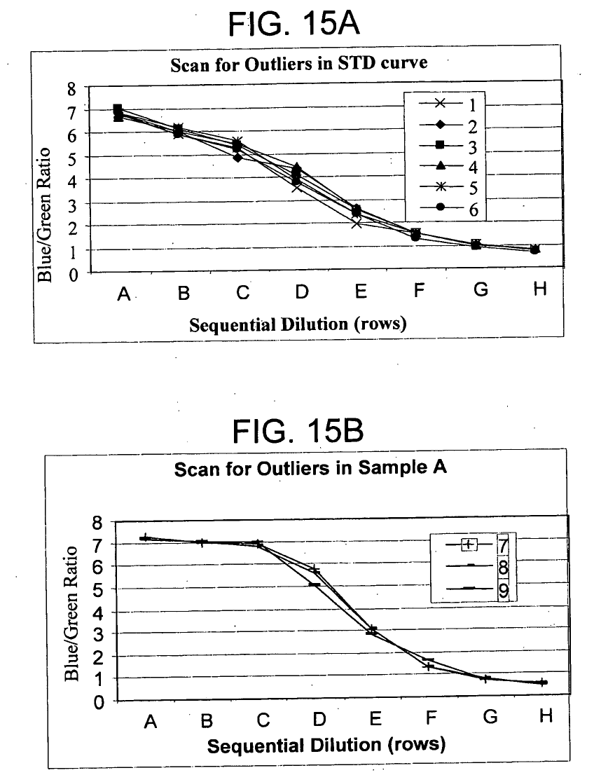

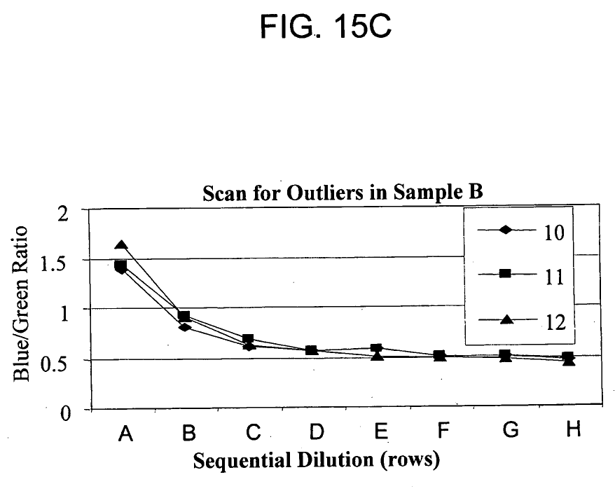

Scroll down and examine the scatter plots for outliers (FIG. 15). Outliers can occur for a variety of reasons, such as dust particles, fingerprints, or pipetting errors. If there are outliers, place the pointer on the data point. The coordinate of the errant point will be displayed. FIG. 15 shows scatter plots which allow for quick identification of outliers that can drastically alter your results. Use common sense. Only eliminate data that is clearly aberrant.

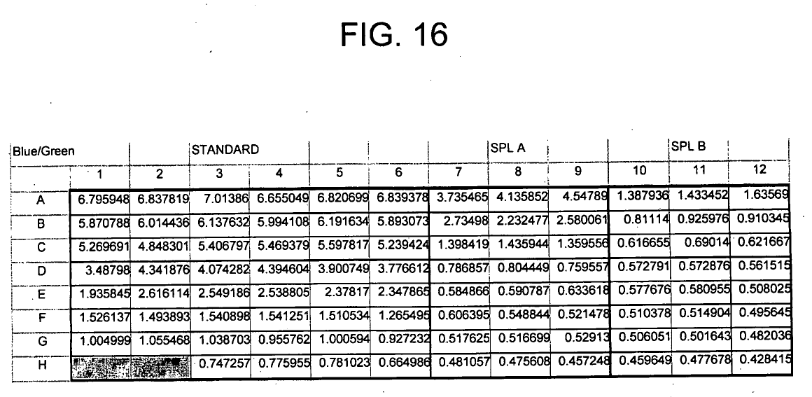

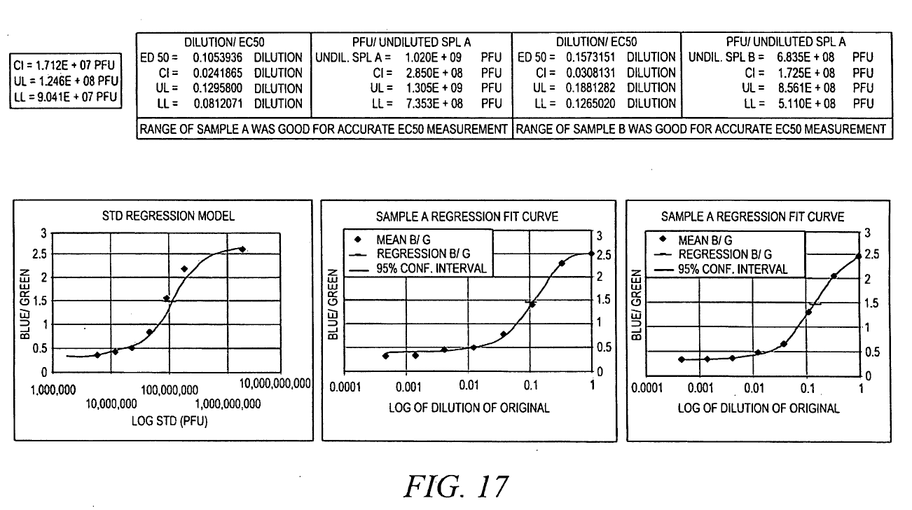

After examining the scatter plots, the contents of cells that correspond to outliers identified from the scatter plots may be cleared. G0 to the average B/G ratio field below the scatter plots (FIG. 16). Highlight the cell(s) that contains the errant data point (i.e., G2), and clear the data from the cell. The point will disappear from the scatter plot (FIG. 15). Once you are satisfied that the data is free of outliers, go to FIG. 17.

The curves for the standard and the unknowns and the titers with confidence limits are displayed in the output tab (see FIG. 17). The first column displays the dilution required to obtain the B/G ration that is ½ maximal. The second column gives the titer and confidence limits. The presentation of this data will be refined in the final version of the tool once placed on our website.

Note: The output tab. The standard curve and curves for the unknowns will be displayed (FIG. 17). The titer is read from the green and orange fields above the curves. The titer is displayed in the well labeled as “Undil. SPLA” (or as “Undil. SPLB”). The 95% confidence interval is a function of the coefficient of variation and is displayed next (CL= . . . ). The upper and lower confidence limits are displayed below the CI.

Appendix 2. Exemplary Quality Control Protocol

Thaw one tube of Titer cells and grow to 100 ml volume in a shake flask (grown in Grace's complete media with 10% heat killed FBS). Thaw one tube of AcMNPV master stock by leaving at room temperature. Keep the tube the dark when not in use. This stock was previously titered by limiting dilution and plaque assay. Infect 50 ml of log phase Sf21 cells at 1.5E6 cells per ml at an MOI of 0.1. Save the remaining virus from the thawed tube at 4 degrees in the dark, and keep the remaining cells growing in log phase. When the infected cell viability drops below 80%, harvest the supernatant. Centrifuge the supernatant at 3000×g for 10 minutes. The clarified supernatant is the standard that will be shipped with the kit. Estimate the titer of the clarified supernatant with log phase titer cells. The thawed master virus will be used as the “standard” and the “build” virus treated as an unknown, in the assay.

Plate the cells at 50,000 cells per well in a black-sided 96 well microtiter plate and allow to attach for at least 1 hour. The thawed master stock virus will now be used as the “standard” virus to measure the titer of the “build” virus. Take a 500 μl aliquot from the clarified supernatant. Remove 50 μl and place in 450 ml of Grace's complete. The undiluted and diluted samples will be read in the assay as unknowns one and two. Dilute the master stock virus and the two “build” virus samples in Grace's complete media according to the standard protocol. Add 10 μl of each dilution to the plate in the afternoon. The following morning, load the cells according to the standard protocol. Read the plate after one hour. Plug the data into the spreadsheet tool and record the titer and coefficient of variation. The coefficient of variation should be less than 20%. If it is not, then the assay needs to be redone. The titer of the build virus needs to be at least 10⁹ pfu or better (10⁸ for the 10× dilution). Check that the titer of the 10× dilution of the build virus is 10% of the undiluted stock. Check the plots for both the master and unknowns. There should be at least two data points above and below the ED₅₀ indicated on the graphs. If these criteria are satisfied, then the virus standard passes QC. Aliquot the build virus, freeze, and re-titer an aliquot of the frozen virus by the same protocol to ensure that the thawed virus has the intended titer. It would also be acceptable to aliquot and freeze the clarified supernatant first, and then do the QC titer assay on a thawed aliquot. This would eliminate doing an extra titer assay but risks the time spent aliquotting if the virus does not have adequate titer.

Appendix 3. Description of the Titer Cell Analysis Tool.

Procedural outline for Double Regression for Sigmoid Curve

### A. DataEntry

1. Data entry: enter Blue values in 96-well plate format
2. Data entry: enter Green values in 96-well plate format
3. Enter titration levels (pfu values for Standard and dilution levels for Spl A and Spl B.
4. Calculate Blue/Green ratios for each well and display
5. Generate curves of B/G ratios grouped by columns for Standard
6. Generate curves of B/G ratios grouped by columns for Spl A and another for Spl B.
7. Allow user interface to omit outlier points from the display

### B. STD Curve, Lower Domain

1. Calculate y-max as average B/G for wells A1-A6, y-min as average for wells H3-H6 and ½ y-max as the average of max-y and min-y.
2. Copy the titer concentrations for the standard curve.
3. Calculate the average y for each row across columns 1-6.
4. Set up an if-then condition to sort all the titers this way: Only avg y values less than ½ y-max will be included in the regression—each of these are designated Y.
5. Tie each Y with the corresponding titer concentrations—each of these are designated X.
6. Begin linear regression on X and Y by the following steps:
   a. Multiply each X by its corresponding Y—each of these are designated XY.
   b. Square each X—each of these are designated X̂2.
   c. Count the number of X terms=n.
   d. Add all X value=SumX
   e. Add all Y values=SumY
   f. Add all XY values=SumXY
   g. Add all XA2 values=SumXA2
   h. F13=SumY/SumX
   i. H13=n/SumX
   j. F14=SumXY/SumX
   k. H14=SumX̂2/SumX
   l. a_{L}=(F13−H13*F14)/(1−H_{13*}H14)
   m. bL=F14−(G15*H14)
   n. Equation: “y exp LD”=(b*X)+a
7. Calculate ED₅₀=(½ y-max−a)/b

### C. STD Curve, Upper Domain

1. Set up an if-then condition that effectively selects all X values that were NOT selected for regression in the Lower Domain.
2. Select all Y values that correspond with these X values.
3. Calculate Log(X) for each X.
4. Designate the maximum value of Log(X) as MaxLogX.
5. Subtract each Log(X) from MaxLogX. E.g., 9.255−8.176=1.079. Designate these as “Log diff fr Max.,” or “LDM.”
6. Calculate the transformed X values as the antilog of LDM(=10̂LDM) and designate these as X′.
7. Subtract each Y value from y-max and designate these as Y′.
8. Perform linear regression on X′ and Y′ values by repeating the steps described above.
   a Regression equation: Y′ exp=aU*X′+bU
9. Calculation of ED₅₀ for Upper Domain:
   a. Subtract ½ y-max from y-max=Y′
   b. Calculate X′=(Y′−a)/b
   c. Calculate Log(X′)
   d. Subtract Log(X′) from the MaxLogX, designate this LogDiffx.
   e. Upper Domain ED₅₀=antilog(LogDiffX)=10̂(LogDiffX).

### D. Standard Curve, Merging Upper and Lower Domains

1. Calc. the average ED₅₀ from Upper and Lower Domains, designate this as Avg ED₅₀.
2. Adjust the Lower Domain regression equation to intersect the Avg ED₅₀ by:
   a. Coefficient “aL” (intercept) remains the same.
   b. Coefficient “bL” (slope) is changed to “bL2”=[(½y-max)−aL)/Avg ED₅₀
   3. Adjust the Upper Domain regression equation to intersect the Avg ED₅₀ by:
   a. Coefficient “aU” (intercept) remains the same.
   b. Coefficient “bU” (slope) is changed to “bu2” by:
      i. Let X′=10̂[MaxLogX−Log(AvgED50)]
      ii. Let Y′=y-max−½y-max
      iii. bU2=(Y′−aU)/X′
4. Adjusted regression equations are:
a Y exp LD=(bL2*X)+bL
b. Y′ exp UD=(bU2*X′)+bU
   E. Standard Curve, Generation of Merged Curve from the Two Adjusted Equations
1. Divide the log range of X in the Standard Titration series into 20 equally spaced log intervals.
2. Using an “if-then” condition, calculate expected Y values when X<Avg ED₅₀ using the adjusted Lower Domain regression equation.
3. For the other Y values (≧AvgED50), calculate the expected Y values for each X by:
   a. Calculate LogDiffX=MaxLogX−Log(X)
   b. Calculate Antilog(LogDiffX), designate as X′
   c. Using the adjusted Upper Domain regression equation, calculate Y′ exp UD.
   d. Calculate Y exp for each X by the formula: Y exp UD=y-max−(Y′exp UD)
4. Plot the Standard Curve using the generated values and smooth curve fit.

### F. Samples A and B Curves and ED50's

1. The procedures for Samples A and B are very similar to those for the Standard curve. Specifically, for each 3-column sample, using average values per dilution level:
   a. Determine Lower Domain ED50_{L}
   b. Determine Upper Domain ED50_{D}
   c. Calculate the Average ED50
2. For calculations involving each sample, use y-max and ½y-max values from the Standard (not from their sample measurements), but use X values of the Samples.
3. Note: For generating the curves to be displayed to the users, it might be best to split the X range into 20 equal log divisions, as in the standard. The same 20 intervals would be used for both samples. This has not yet been done in the Excel model because of the curve-type format for graphing. However, the current Excel fit sometimes does not intersect the ED₅₀ confidence interval at its center, so doing a more complete curve would eliminate that problem.

### G. Confidence Intervals for ED₅₀ Values of Standard:

1. Assign the average of H3-H6 in the 96-well B/G ratios to wells H1 and H2. The data block for calculations in this section encompass A1-H6.
2. Perform a separate linear regression for each column set of Lower Domain data within the data block for calculations. Use the procedure given above for linear regression, but put the following restrictions on this:
   a. Only one deletion of a Y value within any given column of lower domain data is allowed.
   b. At least 2 Y values must be present within a column or an error message will be returned.
3. For each regression equation (up to 6), calculate the ED50_{L} value as described above for the Lower Domain.
4. Calculate the Mean and Standard Deviation of the ED₅₀ values.
5. Calculate CV_{Std}=SD_{Std}/Mean_{Std}.
6. Calculate ½ Confidence interval of ED₅₀ value for the Standard:

½CI_{ED50Std}=AvgED50_{Std}+/−[AvgED50_{std}*t_{0.05, nStd-1}*CV_{Std}/(n_{std})^{0.5}]
7. Calculation of full Confidence Interval of AvgED50_{STD}:
   a. Upper Limit=AvgED50_{std}+CI_{ED50Std}
   b. Lower Limit=AvgED50_{std}−CI_{ED50Std}
8. Plot the CI as a horizontal bar intersecting the curves at the ½y-max value.

### H. Confidence Intervals for ED₅₀ and Original Titer of Sample A:

1. The data block for calculations in this section encompass A7-H9.
2. Repeat steps G.3-G.5 (above) to determine the ED₅₀ values and to calculate the Mean, SD, and CV of Sample A.
3. Calculate the “A-pooled” standard deviation in terms of percent of mean by the following:

CV_{pA}=([(CV_{Std}²)*(n_{Std}−1)+(CV_{SplA}²)*(n_{SplA}−1)]/(n_{Std}+n_{SplA}−2))^{0.5}
4. Degrees of freedom in the t-statistic: df A (n_{std}+n_{SplA}−2). E.g., t_{0.05, 7}=2.365
5. Calculations of ½ Confidence intervals of ED50A:

½CI_{ED50SplA}=AvgED50_{splA}+/−[AvgED50_{splA}*t_{0.05,dfA}*CV_{pA}/(n_{splA})^{0.5}]
6. Calculation of full Confidence Interval of ED50_{SplA}:
   a. Upper Limit=AvgED50_{SplA}+CI_{ED50SplA}
   b. Lower Limit=AvgED50_{SplA}—CI_{ED50SplA}
7. Plot the CI of ED₅₀ as a horizontal bar intersecting the curve at the ½y-max value.
8. Calculate the Concentration of the original undiluted SplA:

Conc_{OrigSplA}=AvgED50_{Std}/AvgED50_{SplA}
9. Calculate the ½Confidence Interval of the original undiluted Sample:

½CI_{OrigSplA}=Conc_{OrigSplA}*[(½CI_{ED50Std}/ED50_{Std})²+(½CI_{ED50SplA}/ED50_{SplA})²]^{0.5}
10. Calculate the Full Confidence Interval of the original undiluted Sample:
   a. Upper Limit=Conc_{OrigSplA}+½CI_{OrigSplA}
   b. Lower Limit=Conc_{OrigSplA}−½CI_{OrigSplA}
11. Report these values (Titer and Confidence Limits) to the user.

### I. Confidence Intervals for ED₅₀ and Original Titer of Sample B:

1. The data block for calculations in this section encompass A10-H12.
2. Repeat steps G.3 to G.5 (above) to determine the ED₅₀ values and to calculate the Mean, SD, and CV of Sample B.
3. Calculate the “B-pooled” standard deviation in terms of percent of mean by the following:

CV_{pB}=([(CV_{Std}²)*(n_{Std}−1)+(CV_{SplB}²)*(n_{SplB}−1)]/(n_{Std}+n_{SplB}−2))^{0.5}
4. Degrees of freedom in the t-statistic: df B=(n_{Std}+n_{SplB}−2). E.g., t_{0.05, 7}=2.365
5. Calculations of ½Confidence intervals of ED50_{B}:

½CI_{ED50SplB}=AvgED50_{splB}+/−[AvgED50_{splB}*t_{0.05,dfB}*CV_{pB}/(n_{splB})^{0.5}]
6. Calculation of full Confidence Interval of ED50_{SplB}:
   a. Upper Limit=AvgED50_{SplB}+CI_{ED50SplB}
   b. Lower Limit=AvgED50_{SplB}−CI_{ED50SplB}
7. Plot the CI of ED₅₀ as a horizontal bar intersecting the curve at the ½y-max value.
8. Calculate the Concentration of the original undiluted SplB:

Conc_{OrigSplB}=AvgED50_{Std}/AvgED50_{SplB}
9. Calculate the ½Confidence Interval of the original undiluted Sample:

½CI_{OrigSplB}=Conc_{OrigSplB}*[(½CI_{ED50Std}/ED50_{Std})²+(½CI_{ED50SplB}/ED50_{SplB)}²]^{0.5}
10. Calculate the Full Confidence Interval of the original undiluted Sample:
   a. Upper Limit=Conc_{OrigSplB}+½CI_{OrigSplB}
   b. Lower Limit=Conc_{OrigSplB}−½CI_{OrigSplB}
11. Report these values (Titer and Confidence Limits) to the user.

Appendix 4: Modifications for the Excel workbook “Regressions™”

Modifications to the methods set out in Apendix 3 and those designed to work with a newer version of the Excel workbook are set out below.

Linear regressions of upper and lower domains were originally unified into a single estimate of ED50 value, also making possible a single smooth sigmoidal curve throughout the low and high domains. In some instances the titer of the working virus stock may not be sufficiently high for an accurate upper domain regression. Therefore, a modification in the calculations of lower domain data alone was made to give an accurate ED50 estimate independent of upper domain data. Linear regression of lower domain data gives a close estimate of ED50 only when the highest y-value in the lower domain is very close to the half maximal y value (“½ Y”) (i.e., in this case the true ED50 would be overestimated by only about 3%). The greater the difference of “½ Y” minus the highest y-value, the greater tends to be the overestimate of the true ED50 value. However, the overestimate reaches a maximum of approximately 22% when this y-difference is equal to or greater than 22% of the full y-scale (Y-max minus Y-min).

Therefore, an algorithm was inserted into the estimate of the ED50 from lower domain data. This algorithm first determines “% Dif Y”, which is the difference between the highest y-value in the lower domain and the half maximal y value (“½ Y”) expressed as the fraction of full y-scale (“Ymax”−“Ymin”). Next, the algorithm identifies if “% Dif Y” is greater than 22%. If so, the ED50 value obtained by linear regression is reduced by 22%. If “% Dif Y” is less than 22%, the ED50 value obtained by linear regression is reduced by the formula: “% Reduction”=(0.863×“% Dif Y”)+3%. This formula was determined empirically by comparing linear regressions and iterative sigmoidal regressions using data generated from the viral titer kit.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

TABLE 3

| | |
|---|---|
| 1 | AAACTTAAGA CTTGCTCTAT TACAATTTTA TTTGTTGGAT CAATTGCATA TTTAATATTA TACAATTCCA TAACGTCCGT TTGATTTAAC GTATAGCTTG |
| | TTTGAATTCT GAACGAGATA ATGTTAAAAT AAACAACCTA GTTAACGTAT AAATTATAAT ATGTTAAGGT ATTGCAGGCA AACTAAATTG CATATCGAAC |
| 101 | CAAATGAATT ATTTAATTAT CAATCATCTT TTACGCGTAC AATTCTACCC GTAAAGCGAG TTTAGTTATG AGCCATGTGC AAAACATGAC ATCAGCTTTT |
| | GTTTACTTAA TAAATTAATA GTTAGTACAA AATGCGCATC TTAAGATGGG CATTTCGCTC AAATCAATAC TCGGTACACG TTTTGTACTG TAGTCGAAAA |
| 201 | ATTTTTATAA CAAATGACAT CATTTCTTGA TTGTGTTTTA CACGTAGAAT TCTACTCGTA AAGCGAGTTC AGTTTTGAAA AACAAATGAC ATCATCTTTT |
| | TAAAAATATT GTTTACTGTA GTAAAGAACT AACACAAAAT GTGCATCTTA AGATGAGCAT TTCGCTCAAG TCAAAACTTT TTGTTTACTG TAGTAGAAAA |
| 301 | TGATTGTGCT TTACAAGTAG AATTCTACCC GTAAATCAAG TTCGGTTTTG AAAAACAAAT GAGTCATATT GTATGATATC ATATTGCAAA CAAATGACTC |
| | ACTAACACGA AATGTTCATC TTAAGATGGG CATTTAGTTC AAGCCAAAAC TTTTTGTTTA CTCAGTATAA CATACTATAG TATAACGTTT GTTTACTGAG |
| 401 | ATCAATCGAT CGTGCGTACA CGTAGAATTC TACTCGTAAA GCGAGTTTAT GAGCCGTGTG CAAAACATGA CATCATCTCG ATTTGAAAAA CAAATGACAT |
| | TAGTTAGCTA GCACGCATGT GCATCTTAAG ATGAGCATTT CGCTCAAATA CTCGGCACAC GTTTTGTACT GTAGTAGAGC TAAACTTTTT GTTTACTGTA |
| 501 | CATCCACTGA TCGTGCGTTA CAAGTAGAAT TCTACTCGTA AAGCCAGTTC GGTTATGAGC CGTGTGCAAA ACATGACATC AGCTTATGAC TCGTACTTGA |
| | GTAGGTGACT AGCACGCAAT GTTCATCTTA AGATGAGCAT TTCGGTCAAG CCAATACTCG GCACACGTTT TGTACTGTAG TCGAATACTG AGCATGAACT |
| 601 | TTGTGTTTTA CGCGTAGAAT TCTACTCGTA AAGCCAGTTC AATTTTAAAA ACAAATGACA TCATCCAAAT TAATAAATGA CAAGCAATGA CAAAATAATA |
| | AACACAAAAT GCGCATCTTA AGATGAGCAT TTCGGTCAAG TTAAAATTTT TGTTTACTGT AGTAGGTTTA ATTATTTACT GTTCGTTACT GTTTTATTAT |
| 701 | TTAGGCAATA AATTTTAACA TTTATTTAAT TGTGTTTAAT ATTACATTTT TGTTGAGTGC ACTAGTCAGT GTGGTGGAAT TGCCCTTAGA ATTTGTCGGG |
| | AATCCGTTAT TTAAAATTGT AAATAAATTA ACACAAATTA TAATGTAAAA ACAACTCACG TGATCAGTCA CACCACCTTA ACGGGAATCT TAAACAGCCC |
| | AcTLP Promoter |
| | ~~~~~~~~~~~~~~~~~~~~~~~~~~ |
| 801 | TCCATTGTCC GTGTGCGCTA GGTACCGAGC TCGGATCCAC TAGTAACGGC CGCCAGTGTG CTGGAATTCG CCCTTTTGCT AGCCCAATTG GCCACTGTTG |
| | AGGTAACAGG CACACGCGAT CCATGGCTCG AGCCTAGGTG ATCATTGCCG GCGGTCACAC GACCTTAAGC GGGAAAACGA TCGGGTTAAC CGGTGACAAC |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 901 | TACGAAATAT CGTCGTCAAC GTGTTTGAAT ACATGTTGGC CCGTACCGTT GGGTAAATCT ATGCATCTGG AGTCGCCGGA ACACTCGTAC TGGTTGTCAG |
| | ATGCTTTATA GCAGCAGTTG CACAAACTTA TGTACAACCG GGCATGGCAA CCCATTTAGA TACGTAGACC TCAGCGGCCT TGTGAGCATG ACCAACAGTC |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1001 | AGTTTCTGAT CCGGTTGATG CACGTTATCA GTTGTGACTC GTTATTATTC AAACATTTGA AATATTGCGT GTCGCCGATA TCGGCCGTTA TGTACGTGTG |
| | TCAAAGACTA GGCCAACTAC GTGCAATAGT CAACACTGAG CAATAATAAG TTTGTAAACT TTATAACGCA CAGCGGCTAT AGCCGGCAAT ACATGCACAC |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1101 | TCCGGCGCCG TTAAACGCGC ACGGATGCGC TTCCACGCAC GACATTAAGT TGCGATCAAA TATTTTATTC GCGGGGCATT CGCCCACCAC GTGGCGCCCA |
| | AGGCCGCGGC AATTTGCGCG TGCCTACGCG AAGGTGCGTG CTGTAATTCA ACGCTAGTTT ATAAAATAAG CGCCCCGTAA GCGGGTGGTG CACCGCGGGT |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1201 | TTTACGCACT GCATAAACTG GTTGACGAGC AAATTGGAGG GAAAGTATGA TAGTATATAG CCGTCTGGCC TGTTTTCACA CAATTCGTTA ACTTTACACT |
| | AAATGCGTGA CGTATTTGAC CAACTGCTCG TTTAACCTCC CTTTCATACT ATCATATATC GGCAGACCGG ACAAAAGTGT GTTAACCAAT TGAAATGTGA |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1301 | GGCCGGTTTC CGCGTCAAAC GTCTAATTAT CTGGACATTC TTCGACTGCG TGCGCTCcGT TTGCAAAACA CCTAAGATAG AACGTGGGAT GATACAAGTG |
| | CCGGCCAAAG GCGCAGTTTG CACATTAATA GACCTGTAAG AAGCTGACGC ACGCGAGGCA AACGTTTTGT GGATTCTATC TTGCACCCTA CTATGTTCAC |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1401 | CGCGTTGGTA GAATAATCTT TGTCCAAGTG TTGGTTCAAC ACCAACGTGT CCAGCAAACG CTCGTCCATG GGATAAAGAC CGGCAGACTT GTTGTCGCAC |
| | GCGCAACCAT CTTATTAGAA ACAGGTTCAC AACCAAGTTG TGGTTGCACA GGTCGTTTGC GAGCAGGTAC CCTATTTCTG GCCGTCTGAA CAACAGCGTG |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1501 | GGCGGCACGG GAACACATTT TAGTTGTGCG TAATCAAAGT TAAAATATGC GGGGCATTTC ATGGTCACGT CGGCCTTGTC GCCGCTCAAA ATAAACTCGT |
| | CCGCCGTGCC CTTGTGTAAA ATCAACACGC ATTAGTTTCA ATTTTATACG CCCCGTAAAG TACCAGTGCA GCCGGAACAG CGGCGAGTTT TATTTGAGCA |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1601 | TGGGATTTTC ATCATTTGCT CTAACGCGAT CGTGTACGAT TCGATCAACA GGTTGAAATT TTTGATTTAA GAAATCAAAA ATTTCAATCC GGTCATCATG |
| | ACCCTAAAAG TAGTAAACGA GATTGCGCTA GCACATGCTA AGCTAGTTGT CCAACTTTAA AAACTAAATT CTTTAGTTTT TAAAGTTAGG CCACTAGTAC |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1701 | CACGCTTTCG TGATAGGTGG AAAGGTCGAC GGTGTTGAAC CACGTTACAA TATAAGTGTT TTGCATAATA TCCGACACGT AGOCTATTAC GTCGGGTGTG |
| | GTGCGAAAGC ACTATCCACC TTTCCAGCTG CCACAACTTG GTGCAATGTT ATATTCACAA AACGTATTAT AGGCTGTGCA TCGGATAATG CAGCCCACAC |
| | AcTLP Promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 1801 | GGTTCGTCTG CGTTGGTGCG CTTCACATAT TCAGTCATCA CTTCGAGCCG CTTGGTCAAA GTCGTTTCGT CAAATTCAAA ATAAATTGCC AAATACATTA |
| | CCAAGCAGAC GCAACCACGC GAAGTGTATA AGTCAGTAGT GAACCTCGGC GAACCACTTT CAGCAAAGCA GTTTAAGTTT TATTTAACGG TTTATGTAAT |
| | AcTLP Promoter beta-lactamase |
| | ~~~~~~~~~~~~~~~~~~~~~~~~~~~~~~~ ~~~~~~~~~~~~~ |
| | M D P E |
| 1901 | AAGTAAACGC TATTATAAGA AAAAAGCTTG GTACCGAGCT CGGATCCACT AGTAACGGCc GCCAGTGTGC TGGAATTCGC CCTTCACCAT GGACCCAGAA |
| | TTCATTTGCG ATAATATTCT TTTTTCGAAC CATGGCTCGA GCCTAGGTGA TCATTGCCGG CGGTCACACG ACCTTAAGCG GGAAGTGGTA CCTGGGTCTT |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | T L V K V K D A E D Q L G A R V G Y I E L D L N S G K I L E S F R P • |
| 2001 | ACGCTGGTGA AAGTAAAAGA TGCTGAAGAT CAGTTGGGTG CCCGAGTGGG TTACATCGAA CTGGATCTCA ACAGCGGTAA GATCCTTGAG AGTTTTCGCC |
| | TGCGACCACT TTCATTTTCT ACGACTTCTA GTCAACCCAC GGGCTCACCC AATGTAGCTT GACCTAGAGT TGTCGCCATT CTAGGAACTC TCAAAAGCGG |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • E E R F P M M S T F K V L L C G A V L S R I D A G Q E Q L G R R I • |
| 2101 | CCGAAGAACG TTTTCCAATG ATGAGCACTT TTAAAGTTCT GCTATGTGGC GCGGTATTAT CCCGTATTGA CGCCGGGCAA GAGCAACTCG GTCGCCGCAT |
| | GGCTTCTTGC AAAAGGTTAC TACTCGTGAA AATTTCAAGA CGATACACCG CGCCATAATA GGGCATAACT GCGGCCCGTT CTCGTTGAGC CAGCGGCGTA |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • H Y S Q N D L V E Y S P V T E K H L T D G M T V R E L C S A A I T |
| 2201 | ACACTATTCT CAGAATGACT TGGTTGAGTA CTCACCAGTC ACAGAAAAGC ATCTTACGGA TGGCATGACA GTAAGAGAAT TATGCAGTGC TGCCATAACC |
| | TGTGATAAGA GTCTTACTGA ACCAACTCAT GAGTGGTCAG TGTCTTTTCG TAGAATGCCT ACCGTACTGT CATTCTCTTA ATACGTCACG ACGGTATTGG |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | M S D N T A A N L L L T T I G G P K E L T A F L H N M G D H V T R L • |
| 2301 | ATGAGTGATA ACACTGCGGC CAACTTACTT CTGACAACGA TCGGAGGACC GAAGGAGCTA ACCGCTTTTT TGCACAACAT GGGGGATCAT GTAACTCGCC |
| | TACTCACTAT TGTGACGCCG GTTGAATGAA GACTGTTGCT AGCCTCCTGG CTTCCTCGAT TGGCGAAAAA ACGTGTTGTA CCCCCTAGTA CATTGAGCGG |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • D R W E P E L N E A I P N D E R D T T M P V A M A T T L R K L L T • |
| 2401 | TTGATCGTTG GGAACCGGAG CTGAATGAAG CCATACCAAA CGACGAGCGT GACACCACGA TGCCTGTAGC AATGGCAACA ACGTTGCGCA AACTATTAAC |
| | AACTAGCAAC CCTTGGCCTC GACTTACTTC GGTATGGTTT GCTGCTCGCA CTGTGGTGCT ACGGACATCG TTACCGTTGT TGCAACGCGT TTGATAATTG |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • G E L L T L A S R Q Q L I D W M E A D K V A G P L L R S A L P A G |
| 2501 | TGGCGAACTA CTTACTCTAG CTTCCCGGCA ACAATTAATA GACTGGATGG AGGCGGATAA AGTTGCAGGA CCACTTCTGC GCTCGGCCCT TCCGGCTGGC |
| | ACCGCTTGAT GAATGAGATC GAAGGGCCGT TGTTAATTAT CTGACCTACC TCCGCCTATT TCAACGTCCT GGTGAAGACG CGAGCCGGGA AGGCCGACCG |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | W F I A D K S G A G E R G S R G I I A A L G P D G K P S R I V V I Y • |
| 2601 | TGGTTTATTG CTGATAAATC TGGAGCCGGT GAGCGTGGGT CTCGCGGTAT CATTGCAGCA CTGGGGCCAG ATGGTAAGCC CTCCCGTATC GTAGTTATCT |
| | ACCAAATAAC GACTATTTAG ACCTCGGCCA CTCGCACCCA GAGCGCCATA GTAACGTCGT GACCCCGGTC TACCATTCGG GAGGGCATAG CATCAATAGA |
| | beta-lactamase |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • T T G S Q A T M D E R N R Q I A E I G A S L I K H W * |
| 2701 | ACACGACGGG GAGTCAGGCA ACTATGGATG AACGAAATAG ACAGATCGCT GAGATAGGTG CCTCACTGAT TAAGCATTGG TAAGATATCG AAGGGCGAAT |
| | TGTGCTGCCC CTCAGTCCGT TGATACCTAC TTGCTTTATC TGTCTAGCGA CTCTATCCAC GGAGTGACTA ATTCGTAACC ATTCTATAGC TTCCCGCTTA |
| 2801 | TCTGCAGATA TCCATCACAC TGGCGGCCGC TCGAGTCTAG ACCCCGAGAT CCCCGACGTT CCCGGCCTTC GCCGCAGTCG CAAGCAGTAA TCAAAACAGC |
| | AGACGTCTAT AGGTAGTGTG ACCGCCGGCG AGCTCAGATC TGGGGCTCTA GGGGCTGCAA GGGCCGGAAG CGGCGTCAGC GTTCGTCATT AGTTTTGTCG |
| 2901 | AAATCGACGG TTTTGAAATA CTCGTACGGC TCTTTGACCA AGTAATAAAA TGCAAGCATC AAAAATATTG CAAAATACAC AAAAAACGTA AGTTCCTTGT. |
| | TTTAGCTGCC AAAACTTTAT GAGCATGCCG AGAAACTGGT TCATTATTTT ACGTTCGTAG TTTTTATAAC GTTTTATGTG TTTTTTGCAT TCAAGGAACA |
| 3001 | GCGCAATAAA GGCCGCAAGG GCCACCGCTG TATTTGTCAA AAATAAACCC GCTATCACCC CATTCAACTT GTTGTTATTT TTGTTCATTG CCAACAACGT |
| | CGCGTTATTT CCGGCGTTCC CGGTGGCGAC ATAAACAGTT TTTATTTGGG CGATAGTGGG GTAAGTTGAA CAACAATAAA AACAAGTAAC GGTTGTTGCA |
| 3101 | GTTTTGCCTG TAAGTGTATT GCATAAACTC GAGACGTGTG TACAGCGAGC TGCTGGCCAG CGCTTGGCCC ACGAGCGTGG CCTCGTCGAA ATCTTTGATC |
| | CAAAACGGAC ATTCACATAA CGTATTTGAG CTCTGCACAC ATGTCGCTCG ACGACCGGTC GCGAACCGGG TGCTCGCACC GGAGCAGCTT TAGAAACTAG |
| | GP64 basal promoter |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| 3201 | TCACCGGTTT GTGTCACGTA GGCCAGATAA CGGTCGGGTA TATAAGATGC CTCAATGCTA CTAGTAAATC AGTCACACCA AGGCTTCAAT AAGGAACACA |
| | AGTGGCCAAA CACAGTGCAT CCGGTCTATT GCCAGCCCAT ATATTCTACG GAGTTACGAT GATCATTTAG TCAGTGTGGT TCCGAAGTTA TTCCTTGTGT |
| | GP64 basal promoter EM7 |
| | ~~~~~~~~ ~~~~~~~~~~~~~~~~~~~~~~~~~~~~~~~~~~~~~~~~~~~~~~~~~~~~~~~~~~~~~~~~~ |
| 3301 | CAAGCAAGCC CTTGACTCAA AGGGCTGCCG GGCTGCAGCA CGTGTTGACA ATTAATCATC GGCATAGTAT ATCGGCATAG TATAATACGA CAAGGTGAGG |
| | GTTCGTTCGG GAACTGAGTT TCCCGACGGC CCGACGTCGT GCACAACTGT TAATTAGTAG CCGTATCATA TAGCCGTATC ATATTATGCT GTTCCACTCC |
| | Blasticidin Resistance |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | M A K P L S Q E E S T L I E R A T A T I N S I P I S E D Y S V • |
| 3401 | AACTAAACCA TGGCCAAGCC TTTGTCTCAA GAAGAATCCA CCCTCATTGA AAGAGCAACG GCTACAATCA ACAGCATCCC CATCTCTGAA GACTACAGCG |
| | TTGATTTGGT ACCGGTTCGG AAACAGAGTT CTTCTTAGGT GGGAGTAACT TTCTCGTTGC CGATGTTAGT TGTCGTAGGG GTAGAGACTT CTGATGTCGC |
| | Blasticidin Resistance |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • A S A A L S S D G R I F T G V N V Y H F T G G P C A E L V V L G T • |
| 3501 | TCGCCAGCGC AGCTCTCTCT AGCGACGGCC GCATCTTCAC TGGTGTCAAT GTATATCATT TTACTGGGGG ACCTTGCGCA GAACTCGTGG TGCTGGGCAC |
| | AGCGGTCGCG TCGAGAGAGA TCGCTGCCGG CGTAGAAGTG ACCACAGTTA CATATAGTAA AATGACCCCC TGGAACGCGT CTTGAGCACC ACGACCCGTG |
| | Blasticidin Resistance |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | • A A A A A A G N L T C I V A I G N E N R G I L S P C G R C R Q V L |
| 3601 | TGCTGCTGCT GCGGCAGCTG GCAACCTGAC TTGTATCGTC GCGATCGGAA ATGAGAACAG GGGCATCTTG AGCCCCTGCG GACGGTGCCG ACAGGTTCTT |
| | ACGACGACGA CGCCGTCGAC CGTTGGACTG AACATAGCAG CGCTAGCCTT TACTCTTGTC CCCGTAGAAC TCGGGGACGC CTGCCACGGC TCTCCAAGAA |
| | Blasticidin Resistance |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | L D L H P G I K A I V K D S D G Q P T A V G I R E L L P S G Y V W E • |
| 3701 | CTCGATCTGC ATCCTGGGAT CAAAGCCATA GTGAAGGACA GTGATGGACA GCCGACGGCA GTTGGGATTC GTGAATTGCT GCCCTCTGGT TATGTGTGGG |
| | GAGCTAGACG TAGGACCCTA GTTTCGGTAT CACTTCCTGT CACTACCTGT CGGCTGCCGT CAACCCTAAG CACTTAACCA CGGGAGACCA ATACACACCC |
| | Blasticidin Resistance |
| | ~~~~~ |
| | • G * |
| 3801 | AGGGCTAAGC ACTTCGTGGC CGAGGAGCAG GACTGACACG TGCTACGAGA TTTCGATTCC ACCGCCGCCT TCTATGAAAG GTTGGGCTTC GGAATCGTTT |
| | TCCCGATTCG TGAAGCACCG GCTCCTCGTC CTGACTGTGC ACGATGCTCT AAAGCTAAGG TGGCGGCGGA ACATACTTTC CAACCCGAAC CCTTAGCAAA |
| 3901 | TCCGGGACGC CGGCTGGATG ATCCTCCAGC GCGGGGATCT CATGCTGGAG TTCTTCGCCC ACCCCAACTT GTTTATTGCA GCTTATAATG GTTACAAATA |
| | AGGCCCTGCG GCCGACCTAC TAGGAGGTCG CGCCCCTAGA GTACGACCTC AAGAAGCGGG TGGGGTTGAA CAAATAACGT CGAATATTAC CAATGTTTAT |
| 4001 | AAGCAATAGC ATCACAAATT TCACAAATAA AGCATTTTTT TCACTGCATT CTAGTTGTGG TTTGTCCAAA CTCATCAATG TATCTTATCA TGTCTGTATA |
| | TTCGTTATCG TAGTGTTTAA AGTGTTTATT TCGTAAAAAA AGTGACGTAA GATCAACACC AAACAGGTTT GAGTAGTTAC ATAGAATAGT ACAGACATAT |
| 4101 | CCGTCGACCT CTAGCTAGAG CTTGGCGTAA TCATGGTCAT AGCTGTTTCC TGTGTGAAAT TGTTATCCGC TCACAATTCC ACACAACATA CGAGCCGGAA |
| | GGCAGCTGGA GATCGATCTC GAACCGCATT AGTACCAGTA TCGACAAAGG ACACACTTTA ACAATAGGCG AGTGTTAAGG TGTGTTGTAT GCTCGGCCTT |
| 4201 | GCATAAAGTG TAAAGCCTGG GGTGCCTAAT GAGTGAGCTA ACTCACATTA ATTGCGTTGC GCTCACTGCC CGCTTTCCAG TCGGGAAACC TGTCGTGCCA |
| | CGTATTTCAC ATTTCGGACC CCACGGATTA CTCACTCGAT TGAGTGTAAT TAACGCAACG CGAGTGACGG GCGAAAGGTC AGCCCTTTGG ACAGCACGGT |
| 4301 | GCTGCATTAA TGAATCGGCC AACGCGCGGG GAGAGGCGGT TTGCGTATTG GGCGCTCTTC C˜CTTCCTCG CTCACTGACT CGCTGCGCTC GGTCGTTCGG |
| | CGACGTAATT ACTTAGCCGG TTGCGCGCCC CTCTCCGCCA AACGCATAAC CCGCGAGAAG GCGAAGGAGC GAGTGACTGA GCGACGCGAG CCAGCAAGCC |
| 4401 | CTGCGGCGAG CGGTATCAGC TCACTCAAAG GCGGTAATAC GGTTATCCAC AGAATCAGGG GATAACGCAG GAAAGAACAT GTGAGCAAAA GGCCAGCAAA |
| | GACGCCGCTC GCCATAGTCG AGTGAGTTTC CGCCATTATC CCAATAGGTG TCTTAGTCCC CTATTGCGTC CTTTCTTGTA CACTCGTTTT CCGGTCGTTT |
| | ~~~~~~~~~~~~~~~~ |
| | pUC ori |
| 4501 | AGGCCAGGAA CCGTAAAAAG GCCGCGTTGC TGGCGTTTTT CCATAGGCTC CGCCCCCCTG ACGAGCATCA CAAAAATCGA CGCTCAAGTC AGAGGTGGCG |
| | TCCGGTCCTT GGCATTTTTC CGGCGCAACG ACCGCAAAAA GGTATCCGAG GCGGGGGGAC TGCTCGTAGT GTTTTTAGCT GCGAGTTCAG TCTCCACCGC |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 4601 | AAACCCGACA GGACTATAAA GATACCAGGC GTTTCCCCCT GGAAGCTCCC TCGTGCGCTC TCCTGTTCCG ACCCTGCCGC TTACCGGATA CCTGTCCGCC |
| | TTTGGGCTGT CCTGATATTT CTATGGTCCG CAAAGGGGGA CCTTCGAGGG AGCACGCGAG AGGACAAGGC TGGCACGGCG AATGGCCTAT GGACAGGCGG |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 4701 | TTTCTCCCTT CGGGAAGCGT GGCGCTTTCT CAATGCTCAC GCTGTAGGTA TCTCAGTTCG GTGTAGGTCG TTCGCTCCAA GCTGGGCTGT GTGCACGAAC |
| | AAAGAGGGAA GCCCTTCGCA CCGCGAAAGA GTTACGAGTG CGACATCCAT AGAGTCAAGC CACATCCAGC AAGCGAGGTT CGACCCGACA CACGTGCTTG |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 4801 | CCCCCGTTCA GCCCGACCGC TGCGCCTTAT CCGGTAACTA TCGTCTTGAG TCCAACCCGG TAAGACACGA CTTATCGCCA CTGGCAGCAG CCACTGGTAA |
| | GGGGGCAAGT CGGGCTGGCG ACGCGGAATA GGCCATTGAT AGCAGAACTC AGGTTGGGCC ATTCTGTGCT GAATAGCGGT GACCGTCGTC GGTGACCATT |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 4901 | CAGGATTAGC AGAGCGAGGT ATGTAGGCGG TGCTACAGAG TTCTTGAAGT GGTGGCCTAA CTACGGCTAC ACTAGAAGGA CAGTATTTGG TATCTGCGCT |
| | GTCCTAATCG TCTCGCTCCA TACATCCGCC ACGATGTCTC AAGAACTTCA CCACCGGATT GATGCCGATG TGATCTTCCT GTCATAAACC ATAGACGCGA |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 5001 | CTGCTGAAGC CAGTTACCTT CGGAAAAAGA GTTGGTAGCT CTTGATCCGG CAAACAAACC ACCGCTGGTA GCGGTGGTTT TTTTGTTTGC AAGCAGCAGA |
| | GACGACTTCG GTCAATGGAA CCCTTTTTCT CAACCATCGA GAACTAGGCC GTTTGTTTGG TGGCGACCAT CGCCACCAAA AAAACAAACG TTCGTCGTCT |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 5101 | TTACGCGCAG AAAAAAAGGA TCTCAAGAAG ATCCTTTGAT CTTTTCTACG GGGTCTGACG CTCAGTGGAA CGAAAACTCA CGTTAAGGGA TTTTGGTCAT |
| | AATGCGCGTC TTTTTTTCCT AGAGTTCTTC TAGGAAACTA GAAAAGATGC CCCAGACTGC GAGTCACCTT GCTTTTGAGT GCAATTCCCT AAAACCAGTA |
| | ~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~~ |
| | pUC ori |
| 5201 | GAGATTATCA AAAAGGATCT TCACCTAGAT CCTTTTAAAT TAAAAATGAA GTTTTAAATC AATCTAAAGT ATATATGAGT AAACTTGGTC TGACAGTTAC |
| | CTCTAATAGT TTTTCCTAGA AGTGGATCTA GGAAAATTTA ATTTTTACTT CAAAATTTAC TTAGATTTCA TATATACTCA TTTGAACCAG ACTGTCAATG |
| 5301 | CAATGCTTAA TCAGTGAGGC ACCTATCTCA GCGATCTGTC TATTTCGTTC ATCCATAGTT GCCTGACTCC CATTATTGAA GCATTTATCA GGGTTATTGT |
| | GTTACGAATT AGTCACTCCG TGGATAGAGT CGCTAGACAG ATAAACCAAG TAGGTATCAA CGGACTGAGG GTAATAACTT CGTAAATAGT CCCAATAACA |
| 5401 | CTCATGAGCG GATACATATT TCAATGTATT TAGAAAAATA AACAAATAGG GCTTCCGCGC ACATTTCCCC GAAAAGTGCC ACCTGACGTC GACGGATCGG |
| | GAGTACTCGC CTATGTATAA ACTTACATAA ATCTTTTTAT TTGTTTATCC CCAACGCGCG TGTAAAGGGG CTTTTCACGG TGGACTGCAG CTGCCTAGCC |
| 5501 | GAGATCCTAG CGTTT |
| | CTCTAGGATC GCAAA |

1. A method of determining the titer of a viral stock, comprising:
(a) contacting insect cells with a sample of the viral stock, wherein the cells comprise a selected nucleic acid sequence operably linked to a transcriptional regulatory sequence that is activated by infection of the cell with a virus; and
(b) quantifying the amount of the selected nucleic acid sequence that is transcribed.

2. The method of claim 1, wherein the insect cells are selected from a group consisting of Lymantria dispar cells, Helicoverpa zea cells, Heliothis virescens cells, Mamestra brassicae cells, Malocosoma disstria cells, Leucania separata cells, Trichoplusia ni cells, Anticarsia gemmatalis cells, Spodoptera exigua cells, Manduca sexta cells, Choristoneura fumiferana cells, Spodoptera frugiperda cells, Bombyx mori cells, Heliothis zea cells, or Estigmene acrea cells.

3. The method of claim 1, wherein the transcriptional regulatory sequence is a viral promoter.

4. The method of claim 3, wherein the viral promoter is from a virus that infects insect cells.

5. The method of claim 3, wherein the promoter is a baculoviral promoter.

6. The method of claim 5, wherein the promoter is from a virus selected from the group consisting of Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Lymantria Dispar Nuclear Polyhedrosis virus (LdMNPV), Bombyx mori S Nuclear Polyhedrosis Virus (BmNPV), Heliothis zea SNPV (HzSnpv), and Trichoplusia ni SNPV (TnSnpv), Plodia interpunctella granulosis virus (PiGV), Trichoplusia ni granulosis virus (TnGV), Pieris brassicae granulosis virus (PbGV), Artogeia rapae granulosis virus (ArGV), Cydia pomonella granulosis virus (CpGV), Heliothis zea NOB (HzNOB), and Oyctes rhinoceros virus.

7. The method of claim 5, wherein the promoter is selected from the group consisting of the lef-3 promoter and the TLP promoter.

8. The method of claim 1, wherein a polypeptide expressed from the selected nucleic acid sequence has an enzymatic activity and quantifying comprises measuring an amount of enzymatic activity.

9. The method of claim 8, wherein the activity is selected from a group consisting of β-lactamase activity, β-galactosidase activity, glucuronidase activity, and luciferase activity.

10. The method of claim 1, wherein a polypeptide expressed from the selected nucleic acid sequence is fluorescent.

11. The method of claim 8, wherein identifying cells in which the selected nucleic acid sequence is transcribed comprises contacting the cells with an enzymatic substrate.

12. A method of monitoring progression of a viral infection in a cell, comprising:
(a) infecting an insect cell with a virus, wherein the cell comprises a selected nucleic acid sequence operably linked to a transcriptional regulatory sequence, wherein the transcriptional regulatory sequence modulates transcription of the selected nucleic acid sequence when the cell is infected with the virus; and
(b) quantifying the amount of the selected nucleic acid sequence that is transcribed.

13. A method of claim 12, wherein a polypeptide having one or more enzymatic activities is encoded by the selected nucleic acid sequence and quantifying comprises determining the amount of enzymatic activity.

14. A method of monitoring a viral infection of a cell population, comprising:
infecting an insect cell population with virus, wherein one or more of the cells of the population comprise a selected nucleic acid sequence operably linked to a transcriptional regulatory sequence, wherein the transcriptional regulatory sequence modulates transcription of the selected nucleic acid sequence when the cell is infected with the virus;
obtaining a sample of the infected cell population; and
quantifying the amount of the selected nucleic acid sequence that is transcribed in the sample.

15. A method of claim 14, wherein cells are selected from a group consisting of Lymantria dispar cells, Helicoverpa zea cells, Heliothis virescens cells, Mamestra brassicae cells, Malocosoma disstria cells, Leucania separata cells, Trichoplusia ni cells, Anticarsia gemmatalis cells, Spodoptera exigua cells, Manduca sexta cells, Choristoneura fumiferana cells, Spodoptera frugiperda cells, Bombyx mori cells, Heliothis zea cells, or Estigmene acrea cells.

16. A method of claim 14, wherein the transcriptional regulatory sequence is a viral promoter.

17. A method of claim 16, wherein the promoter is a baculoviral promoter.

18. A method of claim 16, wherein the promoter is from a virus selected from the group consisting of Autographa californica nuclear polyhedrosis virus (AcMNPV), Choristoneura fumiferana MNPV (CfMNPV), Mamestra brassicae MNPV (MbMNPV), Orgyia pseudotsugata MNPV (OpMNPV), Bombyx mori S Nuclear Polyhedrosis Virus (BmNPV), Heliothis zea SNPV (HzSnpv), Lymantria dispar Nuclear Polyhedrosis Virus (LdMNPV) and Trichoplusia ni SNPV (TnSnpv), Plodia interpunctella granulosis virus (PiGV), Trichoplusia ni granulosis virus (TnGV), Pieris brassicae granulosis virus (PbGV), Artogeia rapae granulosis virus (ArGV), Cydia pomonella granulosis virus (CpGV), Heliothis zea NOB (HzNOB), and Oyctes rhinoceros virus.

19. A method of claim 16, wherein the promoter is selected from a group consisting of the lef-3 promoter and the TLP promoter.

20. A method of claim 13, wherein a polypeptide expressed from the selected nucleic acid sequence has an enzymatic activity and quantifying comprises measuring an amount of enzymatic activity.

21. A method of claim 20, wherein the activity is selected from a group consisting of β-lactamase activity, β-galactosidase activity, glucuronidase activity, and luciferase activity.

22. A method of claim 13, wherein a polypeptide expressed from the selected nucleic acid sequence is fluorescent.

23. A method of claim 21, wherein identifying cells in which the selected nucleic acid sequence is transcribed comprises contacting the cells with an enzymatic substrate.

24-46. (canceled)
